(19) United States
(12) Patent Application Publication
(10) Pub. No.: US 2005/0153402 A1
(43) Pub. Date: Jul. 14, 2005

(54) Corynebacterium glutamicum genes encoding regulatory proteins

(75) Inventors: Markus Pompejus, Waldsee (DE); Burkhard Kroger, Limburgerhof (DE); Hartwig Schroder, Nubloch (DE); Oskar Zelder, Speyer (DE); Gregor Haberhauer, Limburgerhof (DE)

Correspondence Address: LAHIVE & COCKFIELD, LLP., 28 STATE STREET, BOSTON, MA, 02109, US

(73) Assignee: BASF AG, Ludwigshafen (DE)

(21) Appl. No.: 11/006,098

(22) Filed: Dec. 6, 2004

Related U.S. Application Data
(63) Continuation of application No. 09/602,874, filed on Jun. 23, 2000, now abandoned.
(60) Provisional application No. 60/141,031, filed on Jun. 25, 1999.
(60) Provisional application No. 60/142,690, filed on Jul. 1, 1999.
(60) Provisional application No. 60/151,251, filed on Aug. 27, 1999.

(30) Foreign Application Priority Data
Jul. 1, 1999 (DE) 19930476.9
Jul. 8, 1999 (DE) 19931419.5
Jul. 8, 1999 (DE) 19931420.9
Jul. 9, 1999 (DE) 19932122.1
Jul. 9, 1999 (DE) 19932128.0
Jul. 9, 1999 (DE) 19932134.5
Jul. 9, 1999 (DE) 19932206.6
Jul. 9, 1999 (DE) 19932207.4
Jul. 14, 1999 (DE) 19933003.4
Aug. 31, 1999 (DE) 19941390.8
Sep. 3, 1999 (DE) 19942088.2
Sep. 3, 1999 (DE) 19942124.2

(51) Int. Cl. C12P 13/04; C12Q 1/68; C07H 21/04; C12N 9/00; C12N 1/21; C12N 15/74

(52) U.S. Cl. 435/106; 435/6; 435/69.1; 435/183; 435/320.1; 435/252.3; 536/23.2

(57) ABSTRACT

Isolated nucleic acid molecules, designated MR nucleic acid molecules, which encode novel MR proteins from Corynebacterium glutamicum are described. The invention also provides antisense nucleic acid molecules, recombinant expression vectors containing MR nucleic acid molecules, and host cells into which the expression vectors have been introduced. The invention still further provides isolated MR proteins, mutated MR proteins, fusion proteins, antigenic peptides and methods for the improvement of production of a desired compound from C. glutamicum based on genetic engineering of MR genes in this organism.

### RELATED APPLICATIONS

This application claims priority to U.S. Provisional Patent Application No. 60/141,031, filed Jun. 25, 1999, U.S. Provisional Patent Application No. 60/142,690, filed Jul. 1, 1999, and also to U.S. Provisional Patent Application No. 60/151,251, filed Aug. 27, 1999. This application also claims priority to German Patent Application No. 19930476.9, filed Jul. 1, 1999, German Patent Application No. 19931419.5, filed Jul. 8, 1999, German Patent Application No. 1993 1420.9, filed Jul. 8, 1999, German Patent Application No. 19932122.1, filed Jul. 9, 1999, German Patent Application No. 19932128.0, filed Jul. 9, 1999, German Patent Application No. 19932134.5, filed Jul. 9, 1999, German Patent Application No. 19932206.6, filed Jul. 9, 1999, German Patent Application No. 19932207.4, filed Jul. 9, 1999, German Patent Application No. 19933003.4, filed Jul. 14, 1999, German Patent Application No. 19941390.8, filed Aug. 31, 1999, German Patent Application No. 19942088.2, filed Sep. 3, 1999, and German Patent Application No. 19942124.2, filed Sep. 3, 1999. The entire contents of all of the aforementioned applications are hereby expressly incorporated herein by this reference.

### BACKGROUND OF THE INVENTION

Certain products and by-products of naturally-occurring metabolic processes in cells have utility in a wide array of industries, including the food, feed, cosmetics, and pharmaceutical industries. These molecules, collectively termed ‘fine chemicals’, include organic acids, both proteinogenic and non-proteinogenic amino acids, nucleotides and nucleosides, lipids and fatty acids, diols, carbohydrates, aromatic compounds, vitamins and cofactors, and enzymes. Their production is most conveniently performed through the large-scale culture of bacteria developed to produce and secrete large quantities of one or more desired molecules. One particularly useful organism for this purpose is Corynebacterium glutamicum, a gram positive, nonpathogenic bacterium. Through strain selection, a number of mutant strains have been developed which produce an array of desirable compounds. However, selection of strains improved for the production of a particular molecule is a time-consuming and difficult process.

### SUMMARY OF THE INVENTION

The invention provides novel bacterial nucleic acid molecules which have a variety of uses. These uses include the identification of microorganisms which can be used to produce fine chemicals, the modulation of fine chemical production in C. glutamicum or related bacteria, the typing or identification of C. glutamicum or related bacteria, as reference points for mapping the C. glutamicum genome, and as markers for transformation. These novel nucleic acid molecules encode proteins, referred to herein as metabolic regulatory (MR) proteins.

C. glutamicum is a gram positive, aerobic bacterium which is commonly used in industry for the large-scale production of a variety of fine chemicals, and also for the degradation of hydrocarbons (such as in petroleum spills) and for the oxidation of terpenoids. The MR nucleic acid molecules of the invention, therefore, can be used to identify microorganisms which can be used to produce fine chemicals, e.g., by fermentation processes. Modulation of the expression of the MR nucleic acids of the invention, or modification of the sequence of the MR nucleic acid molecules of the invention, can be used to modulate the production of one or more fine chemicals from a microorganism (e.g., to improve the yield or production of one or more fine chemicals from a Corynebacterium or Brevibacterium species).

The MR nucleic acids of the invention may also be used to identify an organism as being Corynebacterium glutamicum or a close relative thereof, or to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present. Although Corynebacterium glutamicum itself is nonpathogenic, it is related to species pathogenic in humans, such as Corynebacterium diphtheriae (the causative agent of diphtheria); the detection of such organisms is of significant clinical relevance.

The MR nucleic acid molecules of the invention may also serve as reference points for mapping of the C. glutamicum genome, or of genomes of related organisms. Similarly, these molecules, or variants or portions thereof, may serve as markers for genetically engineered Corynebacterium or Brevibacterium species. e.g. The MR proteins encoded by the novel nucleic acid molecules of the invention are capable of, for example, performing a function involved in the transcriptional, translational, or posttranslational regulation of proteins important for the normal metabolic functioning of cells. Given the availability of cloning vectors for use in Corynebacterium glutamicum, such as those disclosed in Sinskey et al., U.S. Pat. No. 4,649,119, and techniques for genetic manipulation of C. glutamicum and the related Brevibacterium species (e.g., lactofermentum) (Yoshihama et al, J. Bacteriol. 162: 591-597 (1985); Katsumata et al., J. Bacteriol. 159: 306-311 (1984); and Santamaria et al., J. Gen. Microbiol. 130: 2237-2246 (1984)), the nucleic acid molecules of the invention may be utilized in the genetic engineering of this organism to make it a better or more efficient producer of one or more fine chemicals.

This improved yield, production and/or efficiency of production of a fine chemical may be due to a direct effect of manipulation of a gene of the invention, or it may be due to an indirect effect of such manipulation. Specifically, alterations in C. glutamicum MR proteins which normally regulate the yield, production and/or efficiency of production of a fine chemical metabolic pathways may have a direct impact on the overall production or rate of production of one or more of these desired compounds from this organism. Alterations in the proteins involved in these metabolic pathways may also have an indirect impact on the yield, production and/or efficiency of production of a desired fine chemical. Regulation of metabolism is necessarily complex, and the regulatory mechanisms governing different pathways may intersect at multiple points such that more than one pathway can be rapidly adjusted in accordance with a particular cellular event. This enables the modification of a regulatory protein for one pathway to have an impact on the regulation of many other pathways as well, some of which may be involved in the biosynthesis or degradation of a desired fine chemical. In this indirect fashion, the modulation of action of an MR protein may have an impact on the production of a fine chemical produced by a pathway different from one which that MR protein directly regulates.

The nucleic acid and protein molecules of the invention may be utilized to directly improve the yield, production, and/or efficiency of production of one or more desired fine chemicals from Corynebacterium glutamicum. Using recombinant genetic techniques well known in the art, one or more of the regulatory proteins of the invention may be manipulated such that its function is modulated. For example, the mutation of an MR protein involved in the repression of transcription of a gene encoding an enzyme which is required for the biosynthesis of an amino acid such that it no longer is able to repress transcription may result in an increase in production of that amino acid. Similarly, the alteration of activity of an MR protein resulting in increased translation or activating posttranslational modification of a C. glutamicum protein involved in the biosynthesis of a desired fine chemical may in turn increase the production of that chemical. The opposite situation may also be of benefit: by increasing the repression of transcription or translation, or by posttranslational negative modification of a C. glutamicum protein involved in the regulation of a degradative pathway for a compound, one may increase the production of this chemical. In each case, the overall yield or rate of production of the desired fine chemical may be increased.

It is also possible that such alterations in the protein and nucleotide molecules of the invention may improve the yield, production, and/or efficiency of production of fine chemicals through indirect mechanisms. The metabolism of any one compound is necessarily intertwined with other biosynthetic and degradative pathways within the cell, and necessary cofactors, intermediates, or substrates in one pathway are likely supplied or limited by another such pathway. Therefore, by modulating the activity of one or more of the regulatory proteins of the invention, the production or efficiency of activity of another fine chemical biosynthetic or degradative pathway may be impacted. Further, the manipulation of one or more regulatory proteins may increase the overall ability of the cell to grow and multiply in culture, particularly in large-scale fermentative culture, where growth conditions may be suboptimal. For example, by mutating an MR protein of the invention which would normally cause a repression in the biosynthesis of nucleotides in response to suboptimal extracellular supplies of nutrients (thereby preventing cell division) such that it is decreased in repressor ability, one may increase the biosynthesis of nucleotides and perhaps increase cell division. Changes in MR proteins which result in increased cell growth and division in culture may result in an increase in yield, production, and/or efficiency of production of one or more desired fine chemicals from the culture, due at least to the increased number of cells producing the chemical in the culture.

The invention provides novel nucleic acid molecules which encode proteins, referred to herein as metabolic pathway proteins (MR), which are capable of, for example, performing an enzymatic step involved in the transcriptional, translational, or posttranslational regulation of metabolic pathways in C. glutamicum. Nucleic acid molecules encoding an MR protein are referred to herein as MR nucleic acid molecules. In a preferred embodiment, the MR protein participates in the transcriptional, translational, or posttranslational regulation of one or more metabolic pathways. Examples of such proteins include those encoded by the genes set forth in Table 1.

Accordingly, one aspect of the invention pertains to isolated nucleic acid molecules (e.g., cDNAs, DNAs, or RNAs) comprising a nucleotide sequence encoding an MR protein or biologically active portions thereof, as well as nucleic acid fragments suitable as primers or hybridization probes for the detection or amplification of MR-encoding nucleic acid (e.g., DNA or mRNA). In particularly preferred embodiments, the isolated nucleic acid molecule comprises one of the nucleotide sequences set forth in Appendix A or the coding region or a complement thereof of one of these nucleotide sequences. In other particularly preferred embodiments, the isolated nucleic acid molecule of the invention comprises a nucleotide sequence which hybridizes to or is at least about 50%, preferably at least about 60%, more preferably at least about 70%, 80% or 90%, and even more preferably at least about 95%, 96%, 97%, 98%, 99% or more homologous to a nucleotide sequence set forth in Appendix A, or a portion thereof. In other preferred embodiments, the isolated nucleic acid molecule encodes one of the amino acid sequences set forth in Appendix B. The preferred MR proteins of the present invention also preferably possess at least one of the MR activities described herein.

In another embodiment, the isolated nucleic acid molecule encodes a protein or portion thereof wherein the protein or portion thereof includes an amino acid sequence which is sufficiently homologous to an amino acid sequence of Appendix B, e.g., sufficiently homologous to an amino acid sequence of Appendix B such that the protein or portion thereof maintains an MR activity. Preferably, the protein or portion thereof encoded by the nucleic acid molecule maintains the ability to transcriptionally, translationally, or posttranslationally regulate a metabolic pathway in C. glutamicum. In one embodiment, the protein encoded by the nucleic acid molecule is at least about 50%, preferably at least about 60%, and more preferably at least about 70%, 80%, or 90% and most preferably at least about 95%, 96%, 97%, 98%, or 99% or more homologous to an amino acid sequence of Appendix B (e.g., an entire amino acid sequence selected from those sequences set forth in Appendix B). In another preferred embodiment, the protein is a full length C. glutamicum protein which is substantially homologous to an entire amino acid sequence of Appendix B (encoded by an open reading frame showvn in Appendix A).

In another preferred embodiment, the isolated nucleic acid molecule is derived from C. glutamicum and encodes a protein (e.g., an MR fusion protein) which includes a biologically active domain which is at least about 50% or more homologous to one of the amino acid sequences of Appendix B and is able to transcriptionally, translationally, or posttranslationally regulate a metabolic pathway in C. glutamicum, or has one or more of the activities set forth in Table 1, and which also includes heterologous nucleic acid sequences encoding a heterologous polypeptide or regulatory regions.

In another embodiment, the isolated nucleic acid molecule is at least 15 nucleotides in length and hybridizes under stringent conditions to a nucleic acid molecule comprising a nucleotide sequence of Appendix A. Preferably, the isolated nucleic acid molecule corresponds to a naturally-occurring nucleic acid molecule. More preferably, the isolated nucleic acid encodes a naturally-occurring C. glutamicum MR protein, or a biologically active portion thereof.

Another aspect of the invention pertains to vectors, e.g., recombinant expression vectors, containing the nucleic acid molecules of the invention, and host cells into which such vectors have been introduced. In one embodiment, such a host cell is used to produce an MR protein by culturing the host cell in a suitable medium. The MR protein can be then isolated from the medium or the host cell.

Yet another aspect of the invention pertains to a genetically altered microorganism in which an MR gene has been introduced or altered. In one embodiment, the genome of the microorganism has been altered by introduction of a nucleic acid molecule of the invention encoding wild-type or mutated MR sequence as a transgene. In another embodiment, an endogenous MR gene within the genome of the microorganism has been altered, e.g., functionally disrupted, by homologous recombination with an altered MR gene. In another embodiment, an endogenous or introduced MR gene in a microorganism has been altered by one or more point mutations, deletions, or inversions, but still encodes a functional MR protein. In still another embodiment, one or more of the regulatory regions (e.g., a promoter, repressor, or inducer) of an MR gene in a microorganism has been altered (e.g., by deletion, truncation, inversion, or point mutation) such that the expression of the MR gene is modulated. In a preferred embodiment, the microorganism belongs to the genus Corynebacterium or Brevibacterium, with Corynebacterium glutamicum being particularly preferred. In a preferred embodiment, the microorganism is also utilized for the production of a desired compound, such as an amino acid, with lysine being particularly preferred.

In another aspect, the invention provides a method of identifying the presence or activity of Corynebacterium diphtheriae in a subject. This method includes detection of one or more of the nucleic acid or amino acid sequences of the invention (e.g., the sequences set forth in Appendix A or Appendix B) in a subject, thereby detecting the presence or activity of Corynebacterium diphtheriae in the subject. Still another aspect of the invention pertains to an isolated MR protein or a portion, e.g., a biologically active portion, thereof. In a preferred embodiment, the isolated MR protein or portion thereof transcriptionally, translationally, or posttranslationally regulates one or more metabolic pathways in C. glutamicum . In another preferred embodiment, the isolated MR protein or portion thereof is sufficiently homologous to an amino acid sequence of Appendix B such that the protein or portion thereof maintains the ability to transcriptionally, translationally, or posttranslationally regulate one or more metabolic pathways in C. glutamicum.

The invention also provides an isolated preparation of an MR protein. In preferred embodiments, the MR protein comprises an amino acid sequence of Appendix B. In another preferred embodiment, the invention pertains to an isolated full length protein which is substantially homologous to an entire amino acid sequence of Appendix B (encoded by an open reading frame set forth in Appendix A). In yet another embodiment, the protein is at least about 50%, preferably at least about 60%, and more preferably at least about 70%, 80%, or 90%, and most preferably at least about 95%, 96%, 97%, 98%, or 99% or more homologous to an entire amino acid sequence of Appendix B. In other embodiments, the isolated MR protein comprises an amino acid sequence which is at least about 50% or more homologous to one of the amino acid sequences of Appendix B and is able to transcriptionally, translatiolnally, or posttranslationally regulate one or more metabolic pathways in C. glutamicum, or has one or more of the activities set forth in Table 1.

Alternatively, the isolated MR protein can comprise an amino acid sequence which is encoded by a nucleotide sequence which hybridizes, e.g., hybridizes under stringent conditions, or is at least about 50%, preferably at least about 60%, more preferably at least about 70%, 80%, or 90%, and even more preferably at least about 95%, 96%, 97%, 98,%, or 99% or more homologous, to a nucleotide sequence of Appendix B. It is also preferred that the preferred forms of MR proteins also have one or more of the MR bioactivities described herein.

The MR polypeptide, or a biologically active portion thereof, can be operatively linked to a non-MR polypeptide to form a fusion protein. In preferred embodiments, this fusion protein has an activity which differs from that of the MR protein alone. In other preferred embodiments, this fusion protein transcriptionally, translationally, or posttranslationally regulates one or more metabolic pathways in C. glutamicum. In particularly preferred embodiments, integration of this fusion protein into a host cell modulates production of a desired compound from the cell.

In another aspect, the invention provides methods for screening molecules which modulate the activity of an MR protein, either by interacting with the protein itself or a substrate or binding partner of the MR protein, or by modulating the transcription or translation of an MR nucleic acid molecule of the invention. Another aspect of the invention pertains to a method for producing a fine chemical. This method involves the culturing of a cell containing a vector directing the expression of an MR nucleic acid molecule of the invention, such that a fine chemical is produced. In a preferred embodiment, this method further includes the step of obtaining a cell containing such a vector, in which a cell is transfected with a vector directing the expression of an MR nucleic acid. In another preferred embodiment, this method further includes the step of recovering the fine chemical from the culture. In a particularly preferred embodiment, the cell is from the genus Corynebacterium or Brevibacterium, or is selected from those strains set forth in Table 3.

Another aspect of the invention pertains to methods for modulating production of a molecule from a microorganism. Such methods include contacting the cell with an agent which modulates MR protein activity or MR nucleic acid expression such that a cell associated activity is altered relative to this same activity in the absence of the agent. In a preferred embodiment, the cell is modulated for one or more C. glutamicum metabolic pathway regulatory systems, such that the yields or rate of production of a desired fine chemical by this microorganism is improved. The agent which modulates MR protein activity can be an agent which stimulates MR protein activity or MR nucleic acid expression. Examples of agents which stimulate MR protein activity or MR nucleic acid expression include small molecules, active MR proteins, and nucleic acids encoding MR proteins that have been introduced into the cell. Examples of agents which inhibit MR activity or expression include small molecules and antisense MR nucleic acid molecules.

Another aspect of the invention pertains to methods for modulating yields of a desired compound from a cell, involving the introduction of a wild-type or mutant MR gene into a cell, either maintained on a separate plasmid or integrated into the genome of the host cell. If integrated into the genome, such integration can be random, or it can take place by homologous recombination such that the native gene is replaced by the introduced copy, causing the production of the desired compound from the cell to be modulated. In a preferred embodiment, said yields are increased. In another preferred embodiment, said chemical is a fine chemical. In a particularly preferred embodiment, said fine chemical is an amino acid. In especially preferred embodiments, said amino acid is L-lysine.

### DETAILED DESCRIPTION OF THE INVENTION

The present invention provides MR nucleic acid and protein molecules which are involved in the regulation of metabolism in Corynebacterium glutamicum, including regulation of fine chemical metabolism. The molecules of the invention may be utilized in the modulation of production of fine chemicals from microorganisms, such as C. glutamicum, either directly (e.g., where modulation of the activity of a lysine biosynthesis regulatory protein has a direct impact on the yield, production, and/or efficiency of production of lysine from that organism), or may have an indirect impact which nonetheless results in an increase in yield, production, and/or efficiency of production of the desired compound (e.g., where modulation of the regulation of a nucleotide biosynthesis protein has an impact on the production of an organic acid or a fatty acid from the bacterium, perhaps due to concomitant regulatory alterations in the biosynthetic or degradation pathways for these chemicals in response to the altered regulation of nucleotide biosynthesis). Aspects of the invention are further explicated below.

I. Fine Chemicals

The term ‘fine chemical’ is art-recognized and includes molecules produced by an organism which have applications in various industries, such as, but not limited to, the pharmaceutical, agriculture, and cosmetics industries. Such compounds include organic acids, such as tartaric acid, itaconic acid, and diaminopimelic acid, both proteinogenic and non-proteinogenic amino acids, purine and pyrimidine bases, nucleosides, and nucleotides (as described e.g. in Kuninaka, A. (1996) Nucleotides and related compounds, p. 561-612, in Biotechnology vol. 6, Rehm et al., eds. VCH: Weinheim, and references contained therein), lipids, both saturated and unsaturated fatty acids (e.g., arachidonic acid), diols (e.g., propane diol, and butane diol), carbohydrates (e.g., hyaluronic acid and trehalose), aromatic compounds (e.g., aromatic amines, vanillin, and indigo), vitamins and cofactors (as described in Ullmann's Encyclopedia of Industrial Chemistry, vol. A27, “Vitamins”, p. 443-613 (1996) VCH: Weinheim and references therein; and Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sep. 1-3, 1994 at Penang, Malaysia, AOCS Press, (1995)), enzymes, polyketides (Cane et al. (1998) Science 282: 63-68), and all other chemicals described in Gutcho (1983) Chemicals by Fermentation, Noyes Data Corporation, ISBN: 0818805086 and references therein. The metabolism and uses of certain of these fine chemicals are further explicated below.

A. Amino Acid Metabolism and Uses

Amino acids comprise the basic structural units of all proteins, and as such are essential for normal cellular functioning in all organisms. The term “amino acid” is art-recognized. The proteinogenic amino acids, of which there are 20 species, serve as structural units for proteins, in which they are linked by peptide bonds, while the nonproteinogenic amino acids (hundreds of which are known) are not normally found in proteins (see Ulmann's Encyclopedia of Industrial Chemistry, vol. A2, p. 57-97 VCH: Weinheim (1985)). Amino acids may be in the D- or L- optical configuration, though L-amino acids are generally the only type found in naturally-occurring proteins. Biosynthetic and degradative pathways of each of the 20 proteinogenic amino acids have been well characterized in both prokaryotic and eukaryotic cells (see, for example, Stryer, L. Biochemistry, 3^{rd} edition, pages 578-590 (1988)). The ‘essential’ amino acids (histidine, isoleucine, leucine, lysine, methionine, phenylalanine, threonine, tryptophan, and valine), so named because they are generally a nutritional requirement due to the complexity of their biosyntheses, are readily converted by simple biosynthetic pathways to the remaining 11 ‘nonessential’ amino acids (alanine, arginine, asparagine, aspartate, cysteine, glutamate, glutamine, glycine, proline, serine, and tyrosine). Higher animals do retain the ability to synthesize some of these amino acids, but the essential amino acids must be supplied from the diet in order for normal protein synthesis to occur.

Aside from their function in protein biosynthesis, these amino acids are interesting chemicals in their own right, and many have been found to have various applications in the food, feed, chemical, cosmetics, agriculture, and pharmaceutical industries. Lysine is an important amino acid in the nutrition not only of humans, but also of monogastric animals such as poultry and swine. Glutamate is most commonly used as a flavor additive (mono-sodium glutamate, MSG) and is widely used throughout the food industry, as are aspartate, phenylalanine, glycine, and cysteine. Glycine, L-methionine and tryptophan are all utilized in the pharmaceutical industry. Glutamine, valine, leucine, isoleucine, histidine, arginine, proline, serine and alanine are of use in both the pharmaceutical and cosmetics industries. Threonine, tryptophan, and D/L-methionine are common feed additives. (Leuchtenberger, W. (1996) Amino aids—technical production and use, p. 466-502 in Rehm et al. (eds.) Biotechnology vol. 6, chapter 14a, VCH: Weinheim). Additionally, these amino acids have been found to be useful as precursors for the synthesis of synthetic amino acids and proteins, such as N-acetylcysteine, S-carboxymethyl-L-cysteine, (S)-5-hydroxytryptophan, and others described in Ulmann's Encyclopedia of Industrial Chemistry, vol. A2, p. 57-97, VCH: Weinheim, 1985.

The biosynthesis of these natural amino acids in organisms capable of producing them, such as bacteria, has been well characterized (for review of bacterial amino acid biosynthesis and regulation thereof, see Umbarger, H. E.(1978) Ann. Rev. Biochem. 47: 533-606). Glutamate is synthesized by the reductive amination of α-ketoglutarate, an intermediate in the citric acid cycle. Glutamine, proline, and arginine are each subsequently produced from glutamate. The biosynthesis of serine is a three-step process beginning with 3-phosphoglycerate (an intermediate in glycolysis), and resulting in this amino acid after oxidation, transamination, and hydrolysis steps. Both cysteine and glycine are produced from serine; the former by the condensation of homocysteine with serine, and the latter by the transferal of the side-chain β-carbon atom to tetrahydrofolate, in a reaction catalyzed by serine transhydroxymethylase. Phenylalanine, and tyrosine are synthesized from the glycolytic and pentose phosphate pathway precursors erythrose 4-phosphate and phosphoenolpyruvate in a 9-step biosynthetic pathway that differ only at the final two steps after synthesis of prephenate. Tryptophan is also produced from these two initial molecules, but its synthesis is an 11-step pathway. Tyrosine may also be synthesized from phenylalanine, in a reaction catalyzed by phenylalanine hydroxylase. Alanine, valine, and leucine are all biosynthetic products of pyruvate, the final product of glycolysis. Aspartate is formed from oxaloacetate, an intermediate of the citric acid cycle. Asparagine, methionine, threonine, and lysine are each produced by the conversion of aspartate. Isoleucine is formed from threonine. A complex 9-step pathway results in the production of histidine from 5-phosphoribosyl-1-pyrophosphate, an activated sugar.

Amino acids in excess of the protein synthesis needs of the cell cannot be stored, and are instead degraded to provide intermediates for the major metabolic pathways of the cell (for review see Stryer, L. Biochemistry 3^{rd} ed. Ch. 21 “Amino Acid Degradation and the Urea Cycle” p. 495-516 (1988)). Although the cell is able to convert unwanted amino acids into useful metabolic intermediates, amino acid production is costly in terms of energy, precursor molecules, and the enzymes necessary to synthesize them. Thus it is not surprising that amino acid biosynthesis is regulated by feedback inhibition, in which the presence of a particular amino acid serves to slow or entirely stop its own production (for overview of feedback mechanisms in amino acid biosynthetic pathways, see Stryer, L. Biochemistry, 3^{rd} ed. Ch. 24: “Biosynthesis of Amino Acids and Heme” p. 575-600 (1988)). Thus, the output of any particular amino acid is limited by the amount of that amino acid present in the cell.

B. Vitamin, Cofactor, and Nutraceutical Metabolism and Uses

Vitamins, cofactors, and nutraceuticals comprise another group of molecules which the higher animals have lost the ability to synthesize and so must ingest, although they are readily synthesized by other organisms such as bacteria. These molecules are either bioactive substances themselves, or are precursors of biologically active substances which may serve as electron carriers or intermediates in a variety of metabolic pathways. Aside from their nutritive value, these compounds also have significant industrial value as coloring agents, antioxidants, and catalysts or other processing aids. (For an overview of the structure, activity, and industrial applications of these compounds, see, for example, Ullman's Encyclopedia of Industrial Chemistry, “Vitamins” vol. A27, p. 443-613, VCH: Weinheim, 1996.) The term “vitamin” is art-recognized, and includes nutrients which are required by an organism for normal functioning, but which that organism cannot synthesize by itself The group of vitamins may encompass cofactors and nutraceutical compounds. The language “cofactor” includes nonproteinaceous compounds required for a normal enzymatic activity to occur. Such compounds may be organic or inorganic; the cofactor molecules of the invention are preferably organic. The term “nutraceutical” includes dietary supplements having health benefits in plants and animals, particularly humans. Examples of such molecules are vitamins, antioxidants, and also certain lipids (e.g., polyunsaturated fatty acids).

The biosynthesis of these molecules in organisms capable of producing them, such as bacteria, has been largely characterized (Ullman's Encyclopedia of Industrial Chemistry, “Vitamins” vol. A27, p. 443-613, VCH: Weinheim, 1996; Michal, G. (1999) Biochemical Pathways: An Atlas of Biochemistry and Molecular Biology, John Wiley & Sons; Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sept. 1-3, 1994 at Penang, Malaysia, AOCS Press: Champaign, Ill. X, 374 S).

Thiamin (vitamin B₁) is produced by the chemical coupling of pyrimidine and thiazole moieties. Riboflavin (vitamin B₂) is synthesized from guanosine-5′-triphosphate (GTP) and ribose-5′-phosphate. Riboflavin, in turn, is utilized for the synthesis of flavin mononucleotide (FMN) and flavin adenine dinucleotide (FAD). The family of compounds collectively termed ‘vitamin B₆’ (e.g., pyridoxine, pyridoxamine, pyridoxa-5′-phosphate, and the commercially used pyridoxin hydrochloride) are all derivatives of the common structural unit, 5-hydroxy-6-methylpyridine. Pantothenate (pantothenic acid, (R)-(+)-N-(2,4-dihydroxy-3,3-dimethyl-1-oxobutyl)-β-alanine) can be produced either by chemical synthesis or by fermentation. The final steps in pantothenate biosynthesis consist of the ATP-driven condensation of β-alanine and pantoic acid. The enzymes responsible for the biosynthesis steps for the conversion to pantoic acid, to β-alanine and for the condensation to panthotenic acid are known. The metabolically active form of pantothenate is Coenzyme A, for which the biosynthesis proceeds in 5 enzymatic steps. Pantothenate, pyridoxal-5′-phosphate, cysteine and ATP are the precursors of Coenzyme A. These enzymes not only catalyze the formation of panthothante, but also the production of (R)-pantoic acid, (R)-pantolacton, (R)-panthenol (provitamin B₅), pantetheine (and its derivatives) and coenzyme A.

Biotin biosynthesis from the precursor molecule pimeloyl-CoA in microorganisms has been studied in detail and several of the genes involved have been identified. Many of the corresponding proteins have been found to also be involved in Fe-cluster synthesis and are members of the nifS class of proteins. Lipoic acid is derived from octanoic acid, and serves as a coenzyme in energy metabolism, where it becomes part of the pyruvate dehydrogenase complex and the α-ketoglutarate dehydrogenase complex. The folates are a group of substances which are all derivatives of folic acid, which is turn is derived from L-glutamic acid, p-amino-benzoic acid and 6-methylpterin. The biosynthesis of folic acid and its derivatives, starting from the metabolism intermediates guanosine-5′-triphosphate (GTP), L-glutamic acid and p-amino-benzoic acid has been studied in detail in certain microorganisms.

Corrinoids (such as the cobalamines and particularly vitamin B₁₂) and porphyrines belong to a group of chemicals characterized by a tetrapyrole ring system. The biosynthesis of vitamin B₁₂ is sufficiently complex that it has not yet been completely characterized, but many of the enzymes and substrates involved are now known. Nicotinic acid (nicotinate), and nicotinamide are pyridine derivatives which are also termed ‘niacin’. Niacin is the precursor of the important coenzymes NAD (nicotinamide adenine dinucleotide) and NADP (nicotinamide adenine dinucleotide phosphate) and their reduced forms.

The large-scale production of these compounds has largely relied on cell-free chemical syntheses, though some of these chemicals have also been produced by large-scale culture of microorganisms, such as riboflavin, Vitamin B₆, pantothenate, and biotin. Only Vitamin B₁₂ is produced solely by fermentation, due to the complexity of its synthesis. In vitro methodologies require significant inputs of materials and time, often at great cost.

C. Purine, Pyrimidine, Nucleoside and Nucleotide Metabolism and Uses

Purine and pyrimidine metabolism genes and their corresponding proteins are important targets for the therapy of tumor diseases and viral infections. The language “purine” or “pyrimidine” includes the nitrogenous bases which are constituents of nucleic acids, co-enzymes, and nucleotides. The term “nucleotide” includes the basic structural units of nucleic acid molecules, which are comprised of a nitrogenous base, a pentose sugar (in the case of RNA, the sugar is ribose; in the case of DNA, the sugar is D-deoxyribose), and phosphoric acid. The language “nucleoside” includes molecules which serve as precursors to nucleotides, but which are lacking the phosphoric acid moiety that nucleotides possess. By inhibiting the biosynthesis of these molecules, or their mobilization to form nucleic acid molecules, it is possible to inhibit RNA and DNA synthesis; by inhibiting this activity in a fashion targeted to cancerous cells, the ability of tumor cells to divide and replicate may be inhibited. Additionally, there are nucleotides which do not form nucleic acid molecules, but rather serve as energy stores (i.e., AMP) or as coenzymes (i.e., FAD and NAD).

Several publications have described the use of these chemicals for these medical indications, by influencing purine and/or pyrimidine metabolism (e.g. Christopherson, R. I. and Lyons, S. D. (1990) “Potent inhibitors of de novo pyrimidine and purine biosynthesis as chemotherapeutic agents.” Med. Res. Reviews 10: 505-548). Studies of enzymes involved in purine and pyrimidine metabolism have been focused on the development of new drugs which can be used, for example, as immunosuppressants or anti-proliferants (Smith, J. L., (1995) “Enzymes in nucleotide synthesis.” Curr. Opin. Struct. Biol. 5: 752-757; (1995) Biochem Soc. Transact. 23: 877-902). However, purine and pyrimidine bases, nucleosides and nucleotides have other utilities: as intermediates in the biosynthesis of several fine chemicals (e.g., thiamine, S-adenosyl-methionine, folates, or riboflavin), as energy carriers for the cell (e.g., ATP or GTP), and for chemicals themselves, commonly used as flavor enhancers (e.g., IMP or GMP) or for several medicinal applications (see, for example, Kuninaka, A. (1996) Nucleotides and Related Compounds in Biotechnology vol. 6, Rehm et al., eds. VCH: Weinheim, p. 561-612). Also, enzymes involved in purine, pyrimidine, nucleoside, or nucleotide metabolism are increasingly serving as targets against which chemicals for crop protection, including fungicides, herbicides and insecticides, are developed.

The metabolism of these compounds in bacteria has been characterized (for reviews see, for example, Zalkin, H. and Dixon, J. E. (1992) “de novo purine nucleotide biosynthesis”, in: Progress in Nucleic Acid Research and Molecular Biology, vol. 42, Academic Press:, p. 259-287; and Michal, G. (1999) “Nucleotides and Nucleosides”, Chapter 8 in: Biochemical Pathways: An Atlas of Biochemistry and Molecular Biology, Wiley: New York). Purine metabolism has been the subject of intensive research, and is essential to the normal functioning of the cell. Impaired purine metabolism in higher animals can cause severe disease, such as gout. Purine nucleotides are synthesized from ribose-5-phosphate, in a series of steps through the intermediate compound inosine-5′-phosphate (IMP), resulting in the production of guanosine-5′-monophosphate (GMP) or adenosine-5′-monophosphate (AMP), from which the triphosphate forms utilized as nucleotides are readily formed. These compounds are also utilized as energy stores, so their degradation provides energy for many different biochemical processes in the cell. Pyrimidine biosynthesis proceeds by the formation of uridine-5′-monophosphate (UMP) from ribose-5-phosphate. UMP, in turn, is converted to cytidine-5′-triphosphate (CTP). The deoxy-forms of all of these nucleotides are produced in a one step reduction reaction from the diphosphate ribose form of the nucleotide to the diphosphate deoxyribose form of the nucleotide. Upon phosphorylation, these molecules are able to participate in DNA synthesis.

D. Trehalose Metabolism and Uses

Trehalose consists of two glucose molecules, bound in α,α-1,1 linkage. It is commonly used in the food industry as a sweetener, an additive for dried or frozen foods, and in beverages. However, it also has applications in the pharmaceutical, cosmetics and biotechnology industries (see, for example, Nishimoto el al., (1998) U.S. Pat. No. 5,759,610; Singer, M. A. and Lindquist, S. (1998) Trends Biotech. 16: 460-467; Paiva, C. L. A. and Panek, A. D. (1996) Biotech. Ann. Rev. 2: 293-314; and Shiosaka, M. (1997) J. Japan 172: 97-102). Trehalose is produced by enzymes from many microorganisms and is naturally released into the surrounding medium, from which it can be collected using methods known in the art.

II. Mechanisms of Metabolic Regulation

All living cells have complex catabolic and anabolic metabolic capabilities with many interconnected pathways. In order to maintain a balance between the various parts of this extremely complex metabolic network, the cell employs a finely-tuned regulatory network. By regulating enzyme synthesis and enzyme activity, either independently or simultaneously, the cell is able to control the activity of disparate metabolic pathways to reflect the changing needs of the cell.

The induction or repression of enzyme synthesis may occur at either the level of transcription or translation, or both. Gene expression in prokaryotes is regulated by several mechanisms at the level of transcription (for review see e.g., Lewin, B (1990) Genes IV, Part 3: “Controlling prokaryotic genes by transcription”, Oxford University Press: Oxford, p. 213-301, and references therein, and Michal, G. (1999) Biochemical Pathways: An Atlas of Biochemistry and Molecular Biology, John Wiley & Sons). All such known regulatory processes are mediated by additional genes, which themselves respond to external influences of various kinds (e.g., temperature, nutrient availability, or light). Exemplary protein factors which have been implicated in this type of regulation include the transcription factors. These are proteins which bind to DNA, thereby either increasing the expression of a gene (positive regulation, as in the case of e.g. the ara operon from E. coli) or decreasing gene expression (negative regulation, as in the case of the lac operon from E. coli). These expression-modulating transcription factors can themselves be the subject of regulation. Their activity can, for example, be regulated by the binding of low molecular weight compounds to the DNA-binding protein, thereby stimulating (as in the case of arabinose for the ara operon) or inhibiting (as in the case of the lactose for the lac operon) the binding of these proteins to the appropriate binding site on the DNA (see, for example, Helmann, J. D. and Chamberlin, M. J. (1988) “Structure and function of bacterial sigma factors.” Ann. Rev. Biochem. 57: 839-872; Adhya, S. (1995) “The lac and gal operons today” and Boos, W. et al., “The maltose system.”, both in: Regulation of Gene Expression in Escherichia coli (Lin, E. C. C. and Lynch, A. S., eds.) Chapman & Hall: New York, p. 181-200 and 201-229; and Moran, C. P. (1993) “RNA polymerase and transcription factors.” in: Bacillus subtilis and other gram-positive bacteria, Sonenshein, A. L. et al., eds. ASM: Washington, D.C., p. 653-667.)

Aside from the transcriptional level, protein synthesis is also often regulated at the level of translation. There are multiple mechanisms by which such regulation may occur, including alteration of the ability of the ribosome to bind to one or more mRNAs, binding of the ribosome to the mRNA, the maintenance or removal of mRNA secondary structure, the utilization of common or less common codons for a particular gene, the degree of abundance of one or more tRNAs, and special regulation mechanisms, such as attenuation (see Vellanoweth, R. I. (1993) Translation and its regulation in Bacillus subtilis and other gram-positive bacteria, Sonenshein, A. L. et al., eds. ASM: Washington, D.C., p. 699-711 and references cited therein).

Transcriptional and translational regulation may be targeted to a single protein (sequential regulation) or simultaneously to several proteins in different metabolic pathways (coordinate regulation). Often, genes whose expression is coordinately regulated are physically located near one another in the genome, in an operon or regulon. Such up- or down-regulation of gene transcription and translation is governed by the cellular and extracellular levels of various factors, such as substrates (precursor and intermediate molecules used in one or more metabolic pathways), catabolites (molecules produced by biochemical pathways concerned with the production of energy from the breakdown of complex organic molecules such as sugars), and end products (the molecules resulting at the end of a metabolic pathway). Typically, the expression of genes encoding enzymes necessary for the activity of a particular pathway is induced by high levels of substrate molecules for that pathway. Similarly, such gene expression tends to be repressed when there exist high intracellular levels of the end product of the pathway (Snyder, L. and Champness, W. (1997) The Molecular Biology of Bacteria ASM: Washington). Gene expression may also be regulated by other external and internal factors, such as environmental conditions (e.g., heat, oxidative stress, or starvation). These global environmental changes cause alterations in the expression of specialized modulating genes, which directly or indirectly (via additional genes or proteins) trigger the expression of genes by means of binding to DNA and thereby inducing or repressing transcription (see, for example, Lin, E. C. C. and Lynch, A. S., eds. (1995) Regulation of Gene Expression in Escherichia coli. Chapman & Hall: New York).

Yet another mechanism by which cellular metabolism may be regulated is at the level of the protein. Such regulation is accomplished either by the activities of other proteins, or by binding of low-molecular-weight components which either impede or enable the normal functioning of the protein. Examples of protein regulation by the binding of low-molecular-weight compounds include the binding of GTP or NAD. The binding of a low-molecular-weight chemical is typically reversible, as is the case with the GTP-binding proteins. These proteins exist in two stages (with bound GTP or GDP), one stage being the activated form of the protein, and one stage being inactive.

Regulation of protein activity by the action of other enzymes typically takes the form of covalent modification of the protein (i.e., phosphorylation of amino acid residues such as histidine or aspartate, or methylation). Such covalent modification is typically reversible, as mediated by an enzyme of the opposite activity. An example of this is the opposite activities of kinases and phosphorylases in protein phosphorylation; protein kinases phosphorylate specific residues on a target protein (e.g., serine or threonine), while protein phosphorylases remove phosphate groups from such proteins. Typically, enzymes which modulate the activity of other proteins are themselves modulated by external stimuli. These stimuli are mediated through proteins which function as sensors. A well known mechanism by which such sensor proteins may mediate these external signals is by dimerization, but others are also known (see, for example, Msadek, T. et al. (1993) “Two-Component Regulatory Systems”, in: Bacillus subtilis and Other Gram-Positive Bacteria, Sonenshein, A. L. et al., eds., ASM: Washington p. 729-745 and references cited therein).

A thorough understanding of the regulatory networks governing cellular metabolism in microorganisms is critical for the high-yield production of chemicals by fermentation. Control systems for the down-regulation of metabolic pathways could be removed or lessened to improve the synthesis of desired chemicals, and similarly, those for the up-regulation of metabolic pathways for a desired product could be constitutively activated or optimized in activity (As shown in Hirose, Y. and Okada, H. (1979) “Microbial Production of Amino Acids”, in: Peppler, H. J. and Perlman, D. (eds.) Microbial Technology 2^{nd} ed. Vol. 1, ch. 7 Academic Press: New York.)

III. Elements and Methods of the Invention

The present invention is based, at least in part, on the discovery of novel molecules, referred to herein as MR nucleic acid and protein molecules, which regulate, by transcriptional, translational, or post-translational means, one or more metabolic pathways in C. glutamicum. In one embodiment, the MR molecules transcriptionally, translationally, or posttranslationally regulate a metabolic pathway in C. glutamicum. In a preferred embodiment, the activity of the MR molecules of the present invention to regulate one or more C. glutamicum metabolic pathways has an impact on the production of a desired fine chemical by this organism. In a particularly preferred embodiment, the MR molecules of the invention are modulated in activity, such that the C. glutamicum metabolic pathways which the MR proteins of the invention regulate are modulated in efficiency or output, which either directly or indirectly modulates the yield, production, and/or efficiency of production of a desired fine chemical by C. glutamicum.

The language, “MR protein” or “MR polypeptide” includes proteins which transcriptionally, translationally, or posttranslationally regulate a metabolic pathway in C. glutamicum. Examples of MR proteins include those encoded by the MR genes set forth in Table 1 and Appendix A. The terms “MR gene” or “MR nucleic acid sequence” include nucleic acid sequences encoding an MR protein, which consist of a coding region and also corresponding untranslated 5′ and 3′ sequence regions. Examples of MR genes include those set forth in Table 1. The terms “production” or “productivity” are art-recognized and include the concentration of the fermentation product (for example, the desired fine chemical) formed within a given time and a given fermentation volume (e.g., kg product per hour per liter). The term “efficiency of production” includes the time required for a particular level of production to be achieved (for example, how long it takes for the cell to attain a particular rate of output of a fine chemical). The term “yield” or “product/carbon yield” is art-recognized and includes the efficiency of the conversion of the carbon source into the product (i.e., fine chemical). This is generally written as, for example, kg product per kg carbon source. By increasing the yield or production of the compound, the quantity of recovered molecules, or of useful recovered molecules of that compound in a given amount of culture over a given amount of time is increased. The terms “biosynthesis” or a “biosynthetic pathway” are art-recognized and include the synthesis of a compound, preferably an organic compound, by a cell from intermediate compounds in what may be a multistep and highly regulated process. The terms “degradation” or a “degradation pathway” are art-recognized and include the breakdown of a compound, preferably an organic compound, by a cell to degradation products (generally speaking, smaller or less complex molecules) in what may be a multistep and highly regulated process. The language “metabolism” is art-recognized and includes the totality of the biochemical reactions that take place in an organism. The metabolism of a particular compound, then, (e.g., the metabolism of an amino acid such as glycine) comprises the overall biosynthetic, modification, and degradation pathways in the cell related to this compound. The term, “regulation” is art-recognized and includes the activity of a protein to govern the activity of another protein. The term, “transcriptional regulation” is art-recognized and includes the activity of a protein to impede or activate the conversion of a DNA encoding a target protein to mRNA. The term, “translational regulation” is art-recognized and includes the activity of a protein to impede or activate the conversion of an mRNA encoding a target protein to a protein molecule. The term, “posttranslational regulation” is art-recognized and includes the activity of a protein to impede or improve the activity of a target protein by covalently modifying the target protein (e.g., by methylation, glucosylation, or phosphorylation).

In another embodiment, the MR molecules of the invention are capable of modulating the production of a desired molecule, such as a fine chemical, in a microorganism such as C. glutamicum. Using recombinant genetic techniques, one or more of the regulatory proteins of the invention for metabolic pathways may be manipulated such that its function is modulated. For example, a biosynthetic enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired compound without impairing the viability of the cell. In each case, the overall yield or rate of production of one of these desired fine chemicals may be increased.

It is also possible that such alterations in the protein and nucleotide molecules of the invention may improve the production of fine chemicals in an indirect fashion. The regulatory mechanisms of metabolic pathways in the cell are necessarily intertwined, and the activation of one pathway may lead to the repression or activation of another in a concomitant fashion. Therefore, by modulating the activity of one or more of the proteins of the invention, the production or efficiency of activity of another fine chemical biosynthetic or degradative pathway may be impacted. For example, by decreasing the ability of an MR protein to repress the transcription of a gene encoding a particular amino acid biosynthetic protein, one may concomitantly derepress other amino acid biosynthetic pathways, since these pathways are interrelated. Further, by modifying the MR proteins of the invention, one may uncouple the growth and division of cells from their extracellular surroundings to a certain degree; by impairing an MR protein which normally represses biosynthesis of a nucleotide when the extracellular conditions are suboptimal for growth and cell division such that it now lacks this function, one may permit growth to occur even when the extracellular conditions are poor. This is of particular relevance in large-scale fermentative growth, where conditions within the culture are often suboptimal in terms of temperature, nutrient supply or aeration, but would still support growth and cell division if the cellular regulatory systems for these factors were eliminated.

The isolated nucleic acid sequences of the invention are contained within the genome of a Corynebacterium glutamicum strain available through the American Type Culture Collection, given designation ATCC 13032. The nucleotide sequence of the isolated C. glutamicum MR DNAs and the predicted amino acid sequences of the C. glutamicum MR proteins are shown in Appendices A and B, respectively. Computational analyses were performed which classified and/or identified these nucleotide sequences as sequences which encode metabolic pathway regulatory proteins.

The present invention also pertains to proteins which have an amino acid sequence which is substantially homologous to an amino acid sequence of Appendix B. As used herein, a protein which has an amino acid sequence which is substantially homologous to a selected amino acid sequence is least about 50% homologous to the selected amino acid sequence, e.g., the entire selected amino acid sequence. A protein which has an amino acid sequence which is substantially homologous to a selected amino acid sequence can also be least about 50-60%, preferably at least about 60-70%, and more preferably at least about 70-80%, 80-90%, or 90-95%, and most preferably at least about 96%, 97%, 98%, 99% or more homologous to the selected amino acid sequence.

The MR protein or a biologically active portion or fragment thereof of the invention can transcriptionally, translationally, or posttranslationally regulate a metabolic pathway in C. glutamicum, or have one or more of the activities set forth in Table 1.

Various aspects of the invention are described in further detail in the following subsections:

A. Isolated Nucleic Acid Molecules

One aspect of the invention pertains to isolated nucleic acid molecules that encode MR polypeptides or biologically active portions thereof, as well as nucleic acid fragments sufficient for use as hybridization probes or primers for the identification or amplification of MR-encoding nucleic acid (e.g., MR DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 100 nucleotides of sequence upstream from the 5′ end of the coding region and at least about 20 nucleotides of sequence downstream from the 3′ end of the coding region of the gene. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated MR nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., a C. glutamicum cell). Moreover, an “isolated” nucleic acid molecule, such as a DNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence of Appendix A, or a portion thereof, can be isolated using standard molecular biology techniques and the sequence information provided herein. For example, a C. glutamicum MR DNA can be isolated from a C. glutamicum library using all or portion of one of the sequences of Appendix A as a hybridization probe and standard hybridization techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of one of the sequences of Appendix A can be isolated by the polymerase chain reaction using oligonucleotide primers designed based upon this sequence (e.g., a nucleic acid molecule encompassing all or a portion of one of the sequences of Appendix A can be isolated by the polymerase chain reaction using oligonucleotide primers designed based upon this same sequence of Appendix A). For example, mRNA can be isolated from normal endothelial cells (e.g., by the guanidinium-thiocyanate extraction procedure of Chirgwin et al. (1979) Biochemistry 18: 5294-5299) and DNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Appendix A. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to an MR nucleotide sequence can be prepared by standard synthetic techniques, e.g., using an automated DNA synthesizer.

In a preferred embodiment, an isolated nucleic acid molecule of the invention comprises one of the nucleotide sequences shown in Appendix A. The sequences of Appendix A correspond to the Corynebacterium glutamicum MR DNAs of the invention. This DNA comprises sequences encoding MR proteins (i.e., the “coding region”, indicated in each sequence in Appendix A), as well as 5′ untranslated sequences and 3′ untranslated sequences, also indicated in Appendix A. Alternatively, the nucleic acid molecule can comprise only the coding region of any of the sequences in Appendix A.

For the purposes of this application, it will be understood that each of the sequences set forth in Appendix A has an identifying RXA, RXN, or RXS number having the designation “RXA”, “RXN”, or “RXS” followed by 5 digits (i.e., RXA00603, RXN03181, or RXS00686). Each of these sequences comprises up to three parts: a 5′ upstream region, a coding region, and a downstream region. Each of these three regions is identified by the same RXA, RXN, or RXS designation to eliminate confusion. The recitation “one of the sequences in Appendix A”, then, refers to any of the sequences in Appendix A, which may be distinguished by their differing RXA, RXN, or RXS designations. The coding region of each of these sequences is translated into a corresponding amino acid sequence, which is set forth in Appendix B. The sequences of Appendix B are identified by the same RXA, RXN, or RXS designations as Appendix A, such that they can be readily correlated. For example, the amino acid sequences in Appendix B designated RXA00603, RXN03181, and RXS00686 are translations of the coding regions of the nucleotide sequence of nucleic acid molecules RXA00603, RXN03181, and RXS00686, respectively, in Appendix A. Each of the RXA, RXN, and RXS nucleotide and amino acid sequences of the invention has also been assigned a SEQ ID NO, as indicated in Table 1. For example, as shown in Table 1, the nucleotide sequence of RXA00603 is SEQ ID NO:5 and the amino acid sequence of RXA00603 is SEQ ID NO: 6.

Several of the genes of the invention are “F-designated genes”. An F-designated gene includes those genes set forth in Table 1 which have an ‘F’ in front of the RXA, RXN, or RXS designation. For example, SEQ ID NO:3, designated, as indicated on Table 1, as “F RXA02880”, is an F-designated gene, as are SEQ ID NOs: 21, 27, and 33 (designated on Table 1 as “F RXA02493”, “F RXA00291”, and “F RXA00651”, respectively).

In one embodiment, the nucleic acid molecules of the present invention are not intended to include those compiled in Table 2. In the case of the dapD gene, a sequence for this gene was published in Wehrmann, A., et al. (1998) J. Bacteriol. 180(12): 3159-3165. However, the sequence obtained by the inventors of the present application is significantly longer than the published version. It is believed that the published version relied on an incorrect start codon, and thus represents only a fragment of the actual coding region.

In another preferred embodiment, an isolated nucleic acid molecule of the invention comprises a nucleic acid molecule which is a complement of one of the nucleotide sequences shown in Appendix A, or a portion thereof A nucleic acid molecule which is complementary to one of the nucleotide sequences shown in Appendix A is one which is sufficiently complementary to one of the nucleotide sequences shown in Appendix A such that it can hybridize to one of the nucleotide sequences shown in Appendix A, thereby forming a stable duplex.

In still another preferred embodiment, an isolated nucleic acid molecule of the invention comprises a nucleotide sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to a nucleotide sequence shown in Appendix A, or a portion thereof. Ranges and identity values intermediate to the above-recited ranges, (e.g., 70-90% identical or 80-95% identical) are also intended to be encompassed by the present invention. For example, ranges of identity values using a combination of any of the above values recited as upper and/or lower limits are intended to be included. In an additional preferred embodiment, an isolated nucleic acid molecule of the invention comprises a nucleotide sequence which hybridizes, e.g., hybridizes under stringent conditions, to one of the nucleotide sequences shown in Appendix A, or a portion thereof.

Moreover, the nucleic acid molecule of the invention can comprise only a portion of the coding region of one of the sequences in Appendix A, for example a fragment which can be used as a probe or primer or a fragment encoding a biologically active portion of an MR protein. The nucleotide sequences determined from the cloning of the MR genes from C. glutamicum allows for the generation of probes and primers designed for use in identifying and/or cloning MR homologues in other cell types and organisms, as well as MR homologues from other Corynebacteria or related species. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense strand of one of the sequences set forth in Appendix A, an anti-sense sequence of one of the sequences set forth in Appendix A, or naturally occurring mutants thereof. Primers based on a nucleotide sequence of Appendix A can be used in PCR reactions to clone MR homologues. Probes based on the MR nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme co-factor. Such probes can be used as a part of a diagnostic test kit for identifying cells which misexpress an MR protein, such as by measuring a level of an M4R-encodilng nucleic acid in a sample of cells, e.g., detecting MR mRNA levels or determining whether a genomic MR gene has been mutated or deleted.

In one embodiment, the nucleic acid molecule of the invention encodes a protein or portion thereof which includes an amino acid sequence which is sufficiently homologous to an amino acid sequence of Appendix B such that the protein or portion thereof maintains the ability to transcriptionally, translationally, or posttranslationally regulate a metabolic pathway in C. glutamicum. As used herein, the language “sufficiently homologous” refers to proteins or portions thereof which have amino acid sequences which include a minimum number of identical or equivalent (e.g., an amino acid residue which has a similar side chain as an amino acid residue in one of the sequences of Appendix B) amino acid residues to an amino acid sequence of Appendix B such that the protein or portion thereof is able to transcriptionally, translationally, or posttranslationally regulate a metabolic pathway in C. glutamicum. Protein members of such metabolic pathways, as described herein, may function to regulate the biosynthesis or degradation of one or more fine chemicals. Examples of such activities are also described herein. Thus, “the function of an MR protein” contributes to the overall regulation of one or more fine chemical metabolic pathway, or contributes, either directly or indirectly, to the yield, production, and/or efficiency of production of one or more fine chemicals. Examples of MR protein activities are set forth in Table 1.

In another embodiment, the protein is at least about 50-60%, preferably at least about 60-70%, and more preferably at least about 70-80%, 80-90%, 90-95%, and most preferably at least about 96%, 97%, 98%, 99% or more homologous to an entire amino acid sequence of Appendix B.

Portions of proteins encoded by the MR nucleic acid molecules of the invention are preferably biologically active portions of one of the MR proteins. As used herein, the term “biologically active portion of an MR protein” is intended to include a portion, e.g., a domain/motif, of an MR protein that transcriptionally, translationally, or posttranslationally regulates a metabolic pathway in C. glutamicum, or has an activity as set forth in Table 1. To determine whether an MR protein or a biologically active portion thereof can transcriptionally, translationally, or posttranslationally regulate a metabolic pathway in C. glutamicum, an assay of enzymatic activity may be performed. Such assay methods are well known to those of ordinary skill in the art, as detailed in Example 8 of the Exemplification.

Additional nucleic acid fragments encoding biologically active portions of an MR protein can be prepared by isolating a portion of one of the sequences in Appendix B, expressing the encoded portion of the MR protein or peptide (e.g., by recombinant expression in vitro) and assessing the activity of the encoded portion of the MR protein or peptide.

The invention further encompasses nucleic acid molecules that differ from one of the nucleotide sequences shown in Appendix A (and portions thereof) due to degeneracy of the genetic code and thus encode the same MR protein as that encoded by the nucleotide sequences shown in Appendix A. In another embodiment, an isolated nucleic acid molecule of the invention has a nucleotide sequence encoding a protein having an amino acid sequence shown in Appendix B. In a still further embodiment, the nucleic acid molecule of the invention encodes a full length C. glutamicum protein which is substantially homologous to an amino acid sequence of Appendix B (encoded by an open reading frame shown in Appendix A).

It will be understood by one of ordinary skill in the art that in one embodiment the sequences of the invention are not meant to include the sequences of the prior art, such as those Genbank sequences set forth in Tables 2 or 4 which were available prior to the present invention. In one embodiment, the invention includes nucleotide and amino acid sequences having a percent identity to a nucleotide or amino acid sequence of the invention which is greater than that of a sequence of the prior art (e.g., a Genbank sequence (or the protein encoded by such a sequence) set forth in Tables 2 or 4). For example, the invention includes a nucleotide sequence which is greater than and/or at least 40% identical to the nucleotide sequence designated RXA00603 (SEQ ID NO:5), a nucleotide sequence which is greater than and/or at least 55% identical to the nucleotide sequence designated RXA00129 (SEQ ID NO:29), and a nucleotide sequence which is greater than and/or at least 40% identical to the nucleotide sequence designated RXA00006 (SEQ ID NO:35). One of ordinary skill in the art would be able to calculate the lower threshold of percent identity for any given sequence of the invention by examining the GAP-calculated percent identity scores set forth in Table 4 for each of the three top hits for the given sequence, and by subtracting the highest GAP-calculated percent identity from 100 percent. One of ordinary skill in the art will also appreciate that nucleic acid and amino acid sequences having percent identities greater than the lower threshold so calculated (e.g., at least 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more identical) are also encompassed by the invention.

In addition to the C. glutamicum MR nucleotide sequences shown in Appendix A, it will be appreciated by those of ordinary skill in the art that DNA sequence polymorphisms that lead to changes in the amino acid sequences of MR proteins may exist within a population (e.g., the C. glutamicum population). Such genetic polymorphism in the MR gene may exist among individuals within a population due to natural variation. As used herein, the terms “gene” and “recombinant gene” refer to nucleic acid molecules comprising an open reading frame encoding an MR protein, preferably a C. glutamicum MR protein. Such natural variations can typically result in 1-5% variance in the nucleotide sequence of the MR gene. Any and all such nucleotide variations and resulting amino acid polymorphisms in MR that are the result of natural variation and that do not alter the functional activity of MR proteins are intended to be within the scope of the invention.

Nucleic acid molecules corresponding to natural variants and non-C. glutamicum homologues of the C. glutamicum MR DNA of the invention can be isolated based on their homology to the C. glutamicum MR nucleic acid disclosed herein using the C. glutamicum DNA, or a portion thereof, as a hybridization probe according to standard hybridization techniques under stringent hybridization conditions. Accordingly, in another embodiment, an isolated nucleic acid molecule of the invention is at least 15 nucleotides in length and hybridizes under stringent conditions to the nucleic acid molecule comprising a nucleotide sequence of Appendix A. In other embodiments, the nucleic acid is at least 30, 50, 100, 250 or more nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those of ordinary skill in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to a sequence of Appendix A corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural C. glutamicum MR protein.

In addition to naturally-occurring variants of the MR sequence that may exist in the population, one of ordinary skill in the art will further appreciate that changes can be introduced by mutation into a nucleotide sequence of Appendix A, thereby leading to changes in the amino acid sequence of the encoded MR protein, without altering the functional ability of the MR protein. For example, nucleotide substitutions leading to amino acid substitutions at “non-essential” amino acid residues can be made in a sequence of Appendix A. A “non-essential” amino acid residue is a residue that can be altered from the wild-type sequence of one of the MR proteins (Appendix B) without altering the activity of said MR protein, whereas an “essential” amino acid residue is required for MR protein activity. Other amino acid residues, however, (e.g., those that are not conserved or only semi-conserved in the domain having MR activity) may not be essential for activity and thus are likely to be amenable to alteration without altering MR activity.

Accordingly, another aspect of the invention pertains to nucleic acid molecules encoding MR proteins that contain changes in amino acid residues that are not essential for MR activity. Such MR proteins differ in amino acid sequence from a sequence contained in Appendix B yet retain at least one of the MR activities described herein. In one embodiment, the isolated nucleic acid molecule comprises a nucleotide sequence encoding a protein, wherein the protein comprises an amino acid sequence at least about 50% homologous to an amino acid sequence of Appendix B and is capable of transcriptionally, translationally, or posttranslationally regulating a metabolic pathway in C. glutamicum, or has one or more activities set forth in Table 1. Preferably, the protein encoded by the nucleic acid molecule is at least about 50-60% homologous to one of the sequences in Appendix B, more preferably at least about 60-70% homologous to one of the sequences in Appendix B, even more preferably at least about 70-80%, 80-90%, 90-95% homologous to one of the sequences in Appendix B, and most preferably at least about 96%, 97%, 98%, or 99% homologous to one of the sequences in Appendix B.

To determine the percent homology of two amino acid sequences (e.g., one of the sequences of Appendix B and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences of Appendix B) is occupied by the same amino acid residue or nucleotide as the corresponding position in the other sequence (e.g., a mutant form of the sequence selected from Appendix B), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

An isolated nucleic acid molecule encoding an MR protein homologous to a protein sequence of Appendix B can be created by introducing one or more nucleotide substitutions, additions or deletions into a nucleotide sequence of Appendix A such that one or more amino acid substitutions, additions or deletions are introduced into the encoded protein. Mutations can be introduced into one of the sequences of Appendix A by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an MR protein is preferably replaced with another amino acid residue from the same side chain family. Alternatively, in another embodiment, mutations can be introduced randomly along all or part of an MR coding sequence, such as by saturation mutagenesis, and the resultant mutants can be screened for an MR activity described herein to identify mutants that retain MR activity. Following mutagenesis of one of the sequences of Appendix A, the encoded protein can be expressed recombinantly and the activity of the protein can be determined using, for example, assays described herein (see Example 8 of the Exemplification).

In addition to the nucleic acid molecules encoding MR proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded DNA molecule or complementary to an mRNA sequence. Accordingly, an antisense nucleic acid can hydrogen bond to a sense nucleic acid. The antisense nucleic acid can be complementary to an entire MR coding strand, or to only a portion thereof. In one embodiment, an antisense nucleic acid molecule is antisense to a “coding region” of the coding strand of a nucleotide sequence encoding an MR protein. The term “coding region” refers to the region of the nucleotide sequence comprising codons which are translated into amino acid residues (e.g., the entire codingregion of SEQ ID NO: I (RXN03181) comprises nucleotides 1 to414). In another embodiment, the antisense nucleic acid molecule is antisense to a “noncoding region” of the coding strand of a nucleotide sequence encoding MR. The term “noncoding region” refers to 5′ and 3′ sequences which flank the coding region that are not translated into amino acids (i.e., also referred to as 5′ and 3′ untranslated regions).

Given the coding strand sequences encoding MR disclosed herein (e.g., the sequences set forth in Appendix A), antisense nucleic acids of the invention can be designed according to the rules of Watson and Crick base pairing. The antisense nucleic acid molecule can be complementary to the entire coding region of MR mRNA, but more preferably is an oligonucleotide which is antisense to only a portion of the coding or noncoding region of MR mRNA. For example, the antisense oligonucleotide can be complementary to the region surrounding the translation start site of MR mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylarninomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a cell or generated in situ such that they hybridize with or bind to cellular mRNA and/or genomic DNA encoding an MR protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-59 1)) can be used to catalytically cleave MR mRNA transcripts to thereby inhibit translation of MR mRNA. A ribozyme having specificity for an MR-encoding nucleic acid can be designed based upon the nucleotide sequence of an MR DNA disclosed herein (i.e., SEQ ID NO: 1 (RXN03181 in Appendix A)). For example, a derivative of a Tetrahymena L-19 IVS RNA can be constructed in which the nucleotide sequence of the active site is complementary to the nucleotide sequence to be cleaved in an MR-encoding mRNA. See, e.g., Cech et al. U.S. Pat. No. 4,987,071 and Cech et al. U.S. Pat. No. 5,116,742. Alternatively, MR mRNA can be used to select a catalytic RNA having a specific ribonuclease activity from a pool of RNA molecules. See, e.g., Bartel, D. and Szostak, J. W. (1993) Science 261:1411-1418.

Alternatively, MR gene expression can be inhibited by targeting nucleotide sequences complementary to the regulatory region of an MR nucleotide sequence (e.g., an MR promoter and/or enhancers) to form triple helical structures that prevent transcription of an MR gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

B. Recombinant Expression Vectors and Host Cells

Another aspect of the invention pertains to vectors, preferably expression vectors, containing a nucleic acid encoding an MR protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology. Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, trp-tet-, lpp-, lac-, lpp-lac-, lacI^{q}, T7-, T5-, T3-, gal-, trc-, ara-, SP6-, arny, SPO2, π-P_{R}- or πP_{L}, which are used preferably in bacteria. Additional regulatory sequences are, for example, promoters from yeasts and fungi, such as ADC1, MFα, AC, P-60, CYC1, GAPDH, TEF, rp28, ADH, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., MR proteins, mutant forms of MR proteins, fusion proteins, etc.).

The recombinant expression vectors of the invention can be designed for expression of MR proteins in prokaryotic or eukaryotic cells. For example, MR genes can be expressed in bacterial cells such as C. glutamicum, insect cells (using baculovirus expression vectors), yeast and other fungal cells (see Romanos, M. A. et al. (1992) “Foreign gene expression in yeast: a review”, Yeast 8: 423-488; van den Hondel, C. A. M. J. J. et al. (1991) “Heterologous gene expression in filamentous fungi” in: More Gene Manipulations in Fungi, J. W. Bennet & L. L. Lasure, eds., p. 396-428: Academic Press: San Diego; and van den Hondel, C. A. M. J. J. & Punt, P. J. (1991) “Gene transfer systems and vector development for filamentous fungi, in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) High efficiency Agrobacterium tumefaciens—mediated transformation of Arabidopsis thaliana leaf and cotyledon explants” Plant Cell Rep.: 583-586), or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the MR protein is cloned into a pGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant MR protein unfused to GST can be recovered by cleavage of the fusion protein with thrombin.

Examples of suitable inducible non-fusion E. coli expression vectors include pTrc (Amann et al., (1988) Gene 69:301-315) pLG338, pACYC184, pBR322, pUC18, pUC19, pKC30, pRep4, pHS1, pHS2, pPLc236, pMBL24, pLG200, pUR290, pIN-III 113-B1, πgt11, pBdC1, and pET 11d (Studier et al., Gene Expression Technology. Methods in Enzymology 185, Academic Press, San Diego, Calif. (1990) 60-89; and Pouwels et al., eds. (1985) Cloning Vectors. Elsevier: New York IBSN 0 444 904018). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174(DE3) from a resident π prophage harboring a T7 gn1 gene under the transcriptional control of the lacUV 5 promoter. For transformation of other varieties of bacteria, appropriate vectors may be selected. For example, the plasmids plJ101, plJ364, pIJ702 and pIJ361 are known to be useful in transforming Streptomyces, while plasmids pUB110, pC194, or pBD214 are suited for transformation of Bacillus species. Several plasmids of use in the transfer of genetic information into Corynebacterium include pHM1519, pBL1, pSA77, or pAJ667 (Pouwvels et al., eds. (1985) Cloning Vectors. Elsevier: New York IBSN 0 444 904018).One strategy to maximize recombinant protein expression is to express the protein in a host bacteria with an impaired capacity to proteolytically cleave the recombinant protein (Gottesman, S., Gene Expression Technology. Methods in Enzymnology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as C. glutamicum (Wada et al. (1992) Nucleic Acids Res. 20:2111-2118). Such alteration of nucleic acid sequences of the invention can be carried out by standard DNA synthesis techniques.

In another embodiment, the MR protein expression vector is a yeast expression vector. Examples of vectors for expression in yeast S. cerevisiae include pYepSec1 (Baldari, et al., (1987) Embo J. 6:229-234), 2 μ, pAG-1, Yep6, Yep13, pEMBLYe23, pMFa (Kurjan and Herskowitz, (1982) Cell 30:933-943), pJRY88 (Schultz et al., (1987) Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge, and Pouwels et al., eds. (1985) Cloning Vectors. Elsevier: New York (IBSN 0 444 904018).

Alternatively, the MR proteins of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In another embodiment, the MR proteins of the invention may be expressed in unicellular plant cells (such as algae) or in plant cells from higher plants (e.g., the spermatophytes, such as crop plants). Examples of plant expression vectors include those detailed in: Becker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation”, Nucl. Acid. Res. 12: 8711-8721, and include pLGV23, pGHlac+, pBIN19, pAK2004, and pDH51 (Pouwels el al., eds. (1985) Cloning Vectors. Elsevier: New York IBSN 0 444 904018).

In yet another embodiment, a nucleic acid of the invention is expressed in mammalian cells using a mammalian expression vector. Examples of mammalian expression vectors include pCDM8 (Seed, B. (1987)Nature 329:840) and pMT2PC (Kaufman e al. (1 987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to MR mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisenise genes see Weintraub, H. el al., Antisenise RNA as a molecular tool for genetic analysis, Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an MR protein can be expressed in bacterial cells such as C. glutamicum, insect cells, yeast or mammalian cells (such as Chinese hamster ovary cells (CHO) or COS cells). Other suitable host cells are known to one of ordinary skill in the art. Microorganisms related to Corynebacterium glutamicum which may be conveniently used as host cells for the nucleic acid and protein molecules of the invention are set forth in Table 3.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, plasmid, phagemid, transposon or other DNA) into a host cell, including calcium phosphate or calcium chloride co-precipitation, DEAE-dextran-mediated transfection, lipofection, or electroporation. Suitable methods for transforming or transfecting host cells can be found in Sambrook, el al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an MR protein or can be introduced on a separate vector. Cells stably transfected with the introduced nucleic acid can be identified by, for example, drug selection (e.g., cells that have incorporated the selectable marker gene will survive, while the other cells die).

To create a homologous recombinant microorganism, a vector is prepared which contains at least a portion of an MR gene into which a deletion, addition or substitution has been introduced to thereby alter, e.g., functionally disrupt, the MR gene. Preferably, this MR gene is a Corynebacterium glutamicum MR gene, but it can be a homologue from a related bacterium or even from a mammalian, yeast, or insect source. In a preferred embodiment, the vector is designed such that, upon homologous recombination, the endogenous MR gene is functionally disrupted (i.e., no longer encodes a functional protein; also referred to as a “knock out” vector). Alternatively, the vector can be designed such that, upon homologous recombination, the endogenous MR gene is mutated or otherwise altered but still encodes functional protein (e.g., the upstream regulatory region can be altered to thereby alter the expression of the endogenous MR protein). In the homologous recombination vector, the altered portion of the MR gene is flanked at its 5′ and 3′ ends by additional nucleic acid of the MR gene to allow for homologous recombination to occur between the exogenous MR gene carried by the vector and an endogenous MR gene in a microorganism. The additional flanking MR nucleic acid is of sufficient length for successful homologous recombination with the endogenous gene. Typically, several kilobases of flanking DNA (both at the 5′ and 3′ ends) are included in the vector (see e.g., Thomas, K. R., and Capecchi, M. R. (1987) Cell 51: 503 for a description of homologous recombination vectors). The vector is introduced into a microorganism (e.g., by electroporation) and cells in which the introduced MR gene has homologously recombined with the endogenous MR gene are selected, using art-known techniques.

In another embodiment, recombinant microorganisms can be produced which contain selected systems which allow for regulated expression of the introduced gene.

For example, inclusion of an MR gene on a vector placing it under control of the lac operon permits expression of the MR gene only in the presence of IPTG. Such regulatory systems are well known in the art.

In another embodiment, an endogenous MR gene in a host cell is disrupted (e.g., by homologous recombination or other genetic means known in the art) such that expression of its protein product does not occur. In another embodiment, an endogenous or introduced MR gene in a host cell has been altered by one or more point mutations, deletions, or inversions, but still encodes a functional MR protein. In still another embodiment, one or more of the regulatory regions (e.g., a promoter, repressor, or inducer) of an MR gene in a microorganism has been altered (e.g., by deletion, truncation, inversion, or point mutation) such that the expression of the MR gene is modulated. One of ordinary skill in the art will appreciate that host cells containing more than one of the described MR gene and protein modifications may be readily produced using the methods of the invention, and are meant to be included in the present invention.

A host cell of the invention, such as a prokaryotic or eukaryotic host cell in culture, can be used to produce (i.e., express) an MR protein. Accordingly, the invention further provides methods for producing MR proteins using the host cells of the invention. In one embodiment, the method comprises culturing the host cell of invention (into which a recombinant expression vector encoding an MR protein has been 1 5 introduced, or into which genome has been introduced a gene encoding a wild-type or altered MR protein) in a suitable medium until MR protein is produced. In another embodiment, the method further comprises isolating MR proteins from the medium or the host cell.

C. Isolated MR Proteins

Another aspect of the invention pertains to isolated MR proteins, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of MR protein in which the protein is separated from cellular components of the cells in which it is naturally or recombinantly produced. In one embodiment, the language “substantially free of cellular material” includes preparations of MR protein having less than about 30% (by dry weight) of non-MR protein (also referred to herein as a “contaminating protein”), more preferably less than about 20% of non-MR protein, still more preferably less than about 10% of non-MR protein, and most preferably less than about 5% non-MR protein. When the MR protein or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of MR protein in which the protein is separated from chemical precursors or other chemicals which are involved in the synthesis of the protein. In one embodiment, the language “substantially free of chemical precursors or other chemicals” includes preparations of MR protein having less than about 30% (by dry weight) of chemical precursors or non-MR chemicals, more preferably less than about 20% chemical precursors or non-MR chemicals, still more preferably less than about 10% chemical precursors or non-MR chemicals, and most preferably less than about 5% chemical precursors or noni-MR chemicals. In preferred embodiments, isolated proteins or biologically active portions thereof lack contaminating proteins from the same organism from which the MR protein is derived. Typically, such proteins are produced by recombinant expression of, for example, a C. glutamicum MR protein in a microorganism such as C. glutamicum.

An isolated MR protein or a portion thereof of the invention can transcriptionally, translationally, or posttranslationally regulate a metabolic pathway in C. glutamicum, or has one or more of the activities set forth in Table 1. In preferred embodiments, the protein or portion thereof comprises an amino acid sequence which is sufficiently homologous to an amino acid sequence of Appendix B such that the protein or portion thereof maintains the ability to transcriptionally, translationally, or posttranslationally regulate a metabolic pathway in C. glutamicum. The portion of the protein is preferably a biologically active portion as described herein. In another preferred embodiment, an MR protein of the invention has an amino acid sequence shown in Appendix B. In yet another preferred embodiment, the MR protein has an amino acid sequence which is encoded by a nucleotide sequence which hybridizes, e.g., hybridizes under stringent conditions, to a nucleotide sequence of Appendix A. In still another preferred embodiment, the MR protein has an amino acid sequence which is encoded by a nucleotide sequence that is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to one of the nucleic acid sequences of Appendix A, or a portion thereof. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. The preferred MR proteins of the present invention also preferably possess at least one of the MR activities described herein. For example, a preferred MR protein of the present invention includes an amino acid sequence encoded by a nucleotide sequence which hybridizes, e.g., hybridizes under stringent conditions, to a nucleotide sequence of Appendix A, and which can transcriptionally, translationally, or posttranslationally regulate a metabolic pathway in C. glutamicum, or which has one or more of the activities set forth in Table 1.

In other embodiments, the MR protein is substantially homologous to an amino acid sequence of Appendix B and retains the functional activity of the protein of one of the sequences of Appendix B yet differs in amino acid sequence due to natural variation or mutagenesis, as described in detail in subsection I above. Accordingly, in another-embodiment, the MR protein is a protein which comprises an amino acid sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99%or more homologous to an entire amino acid sequence of Appendix B and which has at least one of the MR activities described herein. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. In another embodiment, the invention pertains to a full length C. glutamicum protein which is substantially homologous to an entire amino acid sequence of Appendix B.

Biologically active portions of an MR protein include peptides comprising amino acid sequences derived from the amino acid sequence of an MR protein, e.g., the an amino acid sequence shown in Appendix B or the amino acid sequence of a protein homologous to an MR protein, which include fewer amino acids than a full length MR protein or the full length protein which is homologous to an MR protein, and exhibit at least one activity of an MR protein. Typically, biologically active portions (peptides, e.g., peptides which are, for example, 5, 10, 15, 20, 30, 35, 36, 37, 38, 39, 40, 50, 100 or more amino acids in length) comprise a domain or motif with at least one activity of an MR protein. Moreover, other biologically active portions, in which other regions of the protein are deleted, can be prepared by recombinant techniques and evaluated for one or more of the activities described herein. Preferably, the biologically active portions of an MR protein include one or more selected domains/motifs or portions thereof having biological activity.

MR proteins are preferably produced by recombinant DNA techniques. For example, a nucleic acid molecule encoding the protein is cloned into an expression vector (as described above), the expression vector is introduced into a host cell (as described above) and the MR protein is expressed in the host cell. The MR protein can then be isolated from the cells by an appropriate purification scheme using standard protein purificationi techniques. Alternative to recombinant expression, an MR protein, polypeptide, or peptide can be synthesized chemically using standard peptide synthesis techniques. Moreover, native MR protein can be isolated from cells (e.g., endothelial cells), for example using an anti-MR antibody, which can be produced by standard techniques utilizing an MR protein or fragment thereof of this invention.

The invention also provides MR chimeric or fusion proteins. As used herein, an MR “chimeric protein” or “fusion protein” comprises an MR polypeptide operatively linked to a non-MR polypeptide. An “MR polypeptide” refers to a polypeptide having an amino acid sequence corresponding to an MR protein, whereas a “non-MR polypeptide” refers to a polypeptide having an amino acid sequence corresponding to a protein which is not substantially homologous to the MR protein, e.g., a protein which is different from the MR protein and which is derived from the same or a different organism. Within the fusion protein, the term “operatively linked” is intended to indicate that the MR polypeptide and the non-MR polypeptide are fused in-frame to each other. The non-MR polypeptide can be fused to the N-terminus or C-terminus of the MR polypeptide. For example, in one embodiment the fusion protein is a GST-MR fusion protein in which the MR sequences are fused to the C-terminus of the GST sequences. Such fusion proteins can facilitate the purification of recombinant MR proteins. In another embodiment, the fusion protein is an MR protein containing a heterologous signal sequence at its N-terminus. In certain host cells (e.g., mammalian host cells), expression and/or secretion of an MR protein can be increased through use of a heterologous signal sequence.

Preferably, an MR chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An MR-encoding nucleic acid can be cloned into such an expression vector such that the fusion moiety is linked in-frame to the MR protein.

Homologues of the MR protein can be generated by mutagenesis, e.g., discrete point mutation or truncation of the MR protein. As used herein, the term “homologue“refers to a variant form of the MR protein which acts as an agonist or antagonist of the activity of the MR protein. An agonist of the MR protein can retain substantially the same, or a subset, of the biological activities of the MR protein. An antagonist of the MR protein can inhibit one or more of the activities of the naturally occurring form of the MR protein, by, for example, competitively binding to a downstream or upstream member of the MR regulatory cascade which includes the MR protein. Thus, the C. glutamicum MR protein and homologues thereof of the present invention may modulate the activity of one or more metabolic pathways which MR proteins regulate in this microorganism.

In an alternative embodiment, homologues of the MR protein can be identified by screening combinatorial libraries of mutants, e.g., truncation mutants, of the MR protein for MR protein agonist or antagonist activity. In one embodiment, a variegated library of MR variants is generated by combinatorial mutagenesis at the nucleic acid level and is encoded by a variegated gene library. A variegated library of MR variants can be produced by, for example, enzymatically ligating a mixture of synthetic oligonucleotides into gene sequences such that a degenerate set of potential MR sequences is expressible as individual polypeptides, or alternatively, as a set of larger fusion proteins (e.g., for phage display) containing the set of MR sequences therein. There are a variety of methods which can be used to produce libraries of potential MR homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential MR sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the MR protein coding can be used to generate a variegated population of MR fragments for screening and subsequent selection of homologues of an MR protein. In one embodiment, a library of coding sequence fragments can be generated by treating a double stranded PCR fragment of an MR coding sequence with a nuclease under conditions wherein nicking occurs only about once per molecule, denaturing the double stranded DNA, renaturing the DNA to form double stranded DNA which can include sense/antisense pairs from different nicked products, remolding single stranded portions from reformed duplexes by treatment with S1 nuclease, and ligating the resulting fragment library into an expression vector. By this method, an expression library can be derived which encodes N-terminal, C-terminal and internal fragments of various sizes of the MR protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of MR homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify MR homologues (Arkin and Yourvan (1992) PNAS 89:7811-7815; Delgrave et al. (1993) Protein Engineering 6(3):327-331).

In another embodiment, cell based assays can be exploited to analyze a variegated MR library, using methods well known in the art.

D. Uses and Methods of the Invention

The nucleic acid molecules, proteins, protein homologues, fusion proteins, primers, vectors, and host cells described herein can be used in one or more of the following methods: identification of C. glutamicum and related organisms; mapping of genomes of organisms related to C. glutamicum; identification and localization of C. glutamicum sequences of interest; evolutionary studies; determination of MR protein regions required for function; modulation of an MR protein activity; modulation of the activity of one or more metabolic pathways; and modulation of cellular production of a desired compound, such as a fine chemical.

The MR nucleic acid molecules of the invention have a variety of uses. First, they may be used to identify an organism as being Corynebacterium glutamicum or a close relative thereof. Also, they may be used to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present.

Although Corynebacterium glutamicum itself is nonpathogenic, it is related to pathogenic species, such as Corynebacterium diphtheriae. Corynebacteriuim diphtheriae is the causative agent of diphtheria, a rapidly developing, acute, febrile infection which involves both local and systemic pathology. In this disease, a local lesion develops in the upper respiratory tract and involves necrotic injury to epithelial cells; the bacilli secrete toxin which is disseminated through this lesion to distal susceptible tissues of the body. Degenerative changes brought about by the inhibition of protein synthesis in these tissues, which include heart, muscle, peripheral nerves, adrenals, kidneys, liver and spleen, result in the systemic pathology of the disease. Diphtheria continues to have high incidence in many parts of the world, including Africa, Asia, Eastern Europe and the independent states of the former Soviet Union. An ongoing epidemic of diphtheria in the latter two regions has resulted in at least 5,000 deaths since 1990.

In one embodiment, the invention provides a method of identifying the presence or activity of Corynebacterium diphtheriae in a subject. This method includes detection of one or more of the nucleic acid or amino acid sequences of the invention (e.g., the sequences set forth in Appendix A or Appendix B) in a subject, thereby detecting the presence or activity of Corynebacterium diphtheriae in the subject. C. glutamicum and C. diphtheriae are related bacteria, and many of the nucleic acid and protein molecules in C. glutamicum are homologous to C. diphtheriae nucleic acid and protein molecules, and can therefore be used to detect C. diphtheriae in a subject.

The nucleic acid and protein molecules of the invention may also serve as markers for specific regions of the genome. This has utility not only in the mapping of the genome, but also for functional studies of C. glutamicum proteins. For example, to identify the region of the genome to which a particular C. glutamicum DNA-binding protein binds, the C. glutamicum genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of C. glutamicum, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related bacteria, such as Brevibacterium lactofermentum.

The MR nucleic acid molecules of the invention are also useful for evolutionary and protein structural studies. The metabolic processes in which the molecules of the invention participate are utilized by a wide variety of prokaryotic and eukaryotic cells; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the MR nucleic acid molecules of the invention may result in the production of MR proteins having functional differences from the wild-type MR proteins. These proteins may be improved in efficiency or activity, may be present in greater numbers in the cell than is usual, or may be decreased in efficiency or activity.

The invention provides methods for screening molecules which modulate the activity of an MR protein, either by interacting with the protein itself or a substrate or binding partner of the MR protein, or by modulating the transcription or translation of an MR nucleic acid molecule of the invention. In such methods, a microorganism expressing one or more MR proteins of the invention is contacted with one or more test compounds, and the effect of each test compound on the activity or level of expression of the MR protein is assessed.

Such changes in activity may directly modulate the yield, production, and/or efficiency of production of one or more fine chemicals from C. glutamicum. For example, by optimizing the activity of an MR protein which activates the transcription or translation of a gene encoding a biosynthetic protein for a desired fine chemical, or by impairing or abrogating the activity of an MR protein which represses the transcription or translation of such a gene, one may also increase the activity or rate of activity of that biosynthetic pathway due to the presence of increased levels of what may have been a limiting enzyme. Similarly, by altering the activity of an MR protein such that it constitutively posttranslationally inactivates a protein involved in a degradation pathway for a desired fine chemical, or by altering the activity of an MR protein such that it constitutively represses the transcription or translation of such a gene, one may increase the yield and/or rate of production of the fine chemical from the cell, due to decreased degradation of the compound.

Further, by modulating the activity of one or more MR proteins, one may indirectly stimulate the production or improve the rate of production of one or more fine chemicals from the cell due to the interrelatedness of disparate metabolic pathways. For example, by increasing the yield, production, and/or efficiency of production by activating the expression of one or more lysine biosynthetic enzymes, one may concomitantly increase the expression of other compounds, such as other amino acids, which the cell would naturally require in greater quantities when lysine is required in greater quantities. Also, regulation of metabolism throughout the cell may be altered such that the cell is better able to grow or replicate under the environmental conditions of fermentative culture (where nutrient and oxygen supplies may be poor and possibly toxic waste products in the environment may be at high levels). For example, by mutagenizing an MR protein which represses the synthesis of molecules necessary for cell membrane production in response to high levels of waste products in the extracellular medium (in order to block cell growth and division in suboptimal growth conditions) such that it no longer is able to repress such synthesis, one may increase the growth and multiplication of the cell in cultures even when the growth conditions are suboptimal. Such enhanced growth or viability should also increase the yields and/or rate of production of a desired fine chemical from fermentative culture, due to the relatively greater number of cells producing this compound in the culture.

The aforementioned mutagenesis strategies for MR proteins to result in increased yields of a fine chemical from C. glutamicum are not meant to be limiting; variations on these strategies will be readily apparent to one of ordinary skill in the art. Using such strategies, and incorporating the mechanisms disclosed herein, the nucleic acid and protein molecules of the invention may be utilized to generate C. glutamicum or related strains of bacteria expressing mutated MR nucleic acid and protein molecules such that the yield and/or efficiency of production of a desired compound is improved. This desired compound may be any natural product of C. glutamicum, which includes the final products of biosynthesis pathways and intermediates of naturally-occurring metabolic pathways, as well as molecules which do not naturally occur in the metabolism of C. glutamicum, but which are produced by a C. glutamicum strain of the invention.

This invention is further illustrated by the following examples which should not be construed as limiting. The contents of all references, patent applications, patents, published patent applications, Tables, Appendices, and the sequence listing cited throughout this application are hereby incorporated by reference.

Exemplification

### EXAMPLE 1

### Preparation of Total Genomic DNA of Corytiebacterium glutamicum ATCC 13032

A culture of Corynebacterium glutamicum (ATCC 13032) was grown overnight at 30° C. with vigorous shaking in BHI medium (Difco). The cells were harvested by centrifugation, the supernatant was discarded and the cells were resuspended in 5 ml buffer-I (5% of the original volume of the culture—all indicated volumes have been calculated for 100 ml of culture volume). Composition of buffer-I: 140.34 g/l sucrose, 2.46 g/l MgSO₄×7H₂O, 10 ml/l KH₂PO₄ solution (100 g/l, adjusted to pH 6.7 with KOH), 50 ml/l M12 concentrate (10 g/l (NH₄)₂SO₄, 1 g/l NaCl, 2 g/l MgSO₄×7H₂O, 0.2 g/l CaCl₂, 0.5 g/l yeast extract (Difco), 10 ml/l trace-elements-mix (200 mg/l FeSO₄ ×H₂O, 10 mg/l ZnSO₄×7 H₂O, 3 mg/l MnCl₂×4 H₂O, 30 mg/l H₃BO₃ 20 mg/l CoCl₂×6 H₂O, 1 mg/l NiCl₂×6 H₂O, 3 mg/l Na₂MoO₄×2 H₂O, 500 mg/l complexing agent (EDTA or critic acid), 100 ml/l vitamins-mix (0.2 mg/l biotin, 0.2 mg/l folic acid, 20 mg/l p-amino benzoic acid, 20 mg/l riboflavin, 40 mg/l ca-panthothenate, 140 mg/l nicotinic acid, 40 mg/l pyridoxole hydrochloride, 200 mg/l myo-inositol). Lysozyme was added to the suspension to a final concentration of 2.5 mg/ml. After an approximately 4 h incubation at 37° C., the cell wall was degraded and the resulting protoplasts are harvested by centrifugation. The pellet was washed once with 5 ml buffer-I and once with 5 ml TE-buffer (10 mM Tris-HCl, 1 mM EDTA, pH 8). The pellet was resuspended in 4 ml TE-buffer and 0.5 ml SDS solution (10%) and 0.5 ml NaCl solution (5 M) are added. After adding of proteinase K to a final concentration of 200 μg/ml, the suspension is incubated for ca. 18 h at 37° C. The DNA was purified by extraction with phenol, phenol-chloroform-isoamylalcohol and chloroform-isoamylalcohol using standard procedures. Then, the DNA was precipitated by adding 1/50 volume of 3 M sodium acetate and 2 volumes of ethanol, followed by a 30 min incubation at −20° C. and a 30 min centrifugation at 12,000 rpm in a high speed centrifuge using a SS34 rotor (Sorvall). The DNA was dissolved in 1 ml TE-buffer containing 20 μg/ml RNaseA and dialysed at 4° C. against 1000 ml TE-buffer for at least 3 hours. During this time, the buffer was exchanged 3 times. To aliquots of 0.4 ml of the dialysed DNA solution, 0.4 ml of 2 M LiCl and 0.8 ml of ethanol are added. After a 30 min incubation at −20° C., the DNA was collected by centrifugation (13,000 rpm, Biofuge Fresco, Heraeus, Hanau, Germany). The DNA pellet was dissolved in TE-buffer. DNA prepared by this procedure could be used for all purposes, including southern blotting or construction of genomic libraries.

### EXAMPLE 2

### Construction of Genomic Libraries in Esclherichia coli of Corynebacterium glutamicum ATCC13032.

Using DNA prepared as described in Example 1, cosmid and plasmid libraries were constructed according to known and well established methods (see e.g., Sambrook, J. el al. (1989) “Molecular Cloning: A Laboratory Manual”, Cold Spring Harbor Laboratory Press, or Ausubel, F. M. el al. (1994) “Current Protocols in Molecular Biology”, John Wiley & Sons.)

Any plasmid or cosmid could be used. Of particular use were the plasmids pBR322 (Sutcliffe, J. G. (1979) Proc. Natl. Acad. Sci. USA, 75:3737-3741); pACYC177 (Change & Cohen (1978) J. Bacteriol 134:1141-1156), plasmids of the pBS series (pBSSK+, pBSSK− and others; Stratagene, LaJolla, USA), or cosmids as SuperCos1 (Stratagene, LaJolla, USA) or Lorist6 (Gibson, T. J., Rosenthal A. and Waterson, R. H. (1987) Gene 53:283-286. Gene libraries specifically for use in C. glutamicum may be constructed using plasmid pSL109 (Lee, H.-S. and A. J. Sinskey (1994) J. Microbiol. Biotechnol. 4: 256-263).

### EXAMPLE 3

### DNA Sequencing and Computational Functional Analysis

Genomic libraries as described in Example 2 were used for DNA sequencing according to standard methods, in particular by the chain termination method using AB1377 sequencing machines (see e.g., Fleischman, R. D. et al. (1995) “Whole-genome Random Sequencing and Assembly of Haemophilus Influenzae Rd., Science, 269:496-512). Sequencing primers with the following nucleotide sequences were used: 5′-GGAAACAGTATGACCATG-3′ or 5′-GTAAAACGACGGCCAGT-3′.

### EXAMPLE 4

### In Vivo Mutagenesis

In vivo mutagenesis of Corynebacterium glutamicum can be performed by passage of plasmid (or other vector) DNA through E. coli or other microorganisms (e.g. Bacillus spp. or yeasts such as Saccharomyces cerevisiae) which are impaired in their capabilities to maintain the integrity of their genetic information. Typical mutator strains have mutations in the genes for the DNA repair system (e.g., mutHLS, mutD, mutT, etc.; for reference, see Rupp, W. D. (1996) DNA repair mechanisms, in: Escherichia coli and Salmonella, p. 2277-2294, ASM: Washington.) Such strains are well known to one of ordinary skill in the art. The use of such strains is illustrated, for example, in Greener, A. and Callahan, M. (1994) Strategies 7: 32-34.

### EXAMPLE 5

### DNA Transfer Between Escherichia coli and Corynebacterium glutamicum

Several Corynebacterium and Brevibacterium species contain endogenous plasmids (as e.g., pHM1519 or pBL1) which replicate autonomously (for review see, e.g., Martin, J. F. el al. (1987) Biotechnology, 5:137-146). Shuttle vectors for Escherichia coli and Corynebacterium glutamicum can be readily constructed by using standard vectors for E. coli (Sambrook, J. et al. (1989), “Molecular Cloning: A Laboratory Manual”, Cold Spring Harbor Laboratory Press or Ausubel, F. M. el al. (1994) “Current Protocols in Molecular Biology”, John Wiley & Sons) to which a origin or replication for and a suitable marker from Corynebacterium glutamicum is added. Such origins of replication are preferably taken from endogenous plasmids isolated from Corynebacterium and Brevibacterium species. Of particular use as transformation markers for these species are genes for kanamycin resistance (such as those derived from the Tn5 or Tn903 transposons) or chloramphenicol (Winnacker, E. L. (1987) “From Genes to Clones—Introduction to Gene Technology, VCH, Weinheim). There are numerous examples in the literature of the construction of a wide variety of shuttle vectors which replicate in both E. coli and C. glutamicum, and which can be used for several purposes, including gene over-expression (for reference, see e.g., Yoshihama, M. et al. (1985) J. Bacteriol. 162:591-597, Martin J. F. et al. (1987) Biotechnology, 5:137-146 and Eikmanns, B. J. et al. (1991) Gene, 102:93-98).

Using standard methods, it is possible to clone a gene of interest into one of the shuttle vectors described above and to introduce such a hybrid vectors into strains of Corynebacterium glutamicum. Transformation of C. glutamicum can be achieved by protoplast transformation (Kastsumata, R. et al. (1984) J. Bacteriol. 159306-311), electroporation (Liebl, E. et al. (1989) FEMS Microbiol. Letters, 53 :399-303) and in cases where special vectors are used, also by conjugation (as described e.g. in Schäfer, A et al. (1990) J. Bacteriol. 172:1663 -1666). It is also possible to transfer the shuttle vectors for C. glutamicum to E. coli by preparing plasmid DNA from C. glutamicum (using standard methods well-known in the art) and transforming it into E. coli. This transformation step can be performed using standard methods, but it is advantageous to use an Mcr-deficient E. coli strain, such as NM522 (Gough & Murray (1983) J. Mol. Biol. 166:1-19).

Genes may be overexpressed in C. glutamicum strains using plasmids which comprise pCG1 (U.S. Pat. No. 4,617,267) or fragments thereof, and optionally the gene for kanamycin resistance from TN903 (Grindley, N. D. and Joyce, C. M. (1980) Proc. Natl. Acad. Sci. USA 77(12): 7176-7180). In addition, genes may be overexpressed in C. glutamicum strains using plasmid pSL109 (Lee, H.-S. and A. J. Sinskey (1994) J. Microbiol. Biotechnol. 4: 256-263).

Aside from the use of replicative plasmids, gene overexpression can also be achieved by integration into the genome. Genomic integration in C. glutamicum or other Corynebacterium or Brevibacterium species may be accomplished by well-known methods, such as homologous recombination with genomic region(s), restriction endonuclease mediated integration (REMI) (see, e.g., DE Patent 19823834), or through the use of transposons. It is also possible to modulate the activity of a gene of interest by modifying the regulatory regions (e.g., a promoter, a repressor, and/or an enhancer) by sequence modification, insertion, or deletion using site-directed methods (such as homologous recombination) or methods based on random events (such as transposon mutagenesis or REMI). Nucleic acid sequences which function as transcriptional terminators may also be inserted 3′ to the coding region of one or more genes of the invention; such terminators are well-known in the art and are described, for example, in Winnacker, E. L. (1987) From Genes to Clones—Introduction to Gene Technology. VCH: Weinheim.

### EXAMPLE 6

### Assessment of the Expression of the Mutant Protein

Observations of the activity of a mutated protein in a transformed host cell rely on the fact that the mutant protein is expressed in a similar fashion and in a similar quantity to that of the wild-type protein. A useful method to ascertain the level of transcription of the mutant gene (an indicator of the amount of mRNA available for translation to the gene product) is to perform a Northern blot (for reference see, for example, Ausubel et al. (1988) Current Protocols in Molecular Biology, Wiley: N.Y.), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information is evidence of the degree of transcription of the mutant gene. Total cellular RNA can be prepared from Corynebacterium glutamicum by several methods, all well-known in the art, such as that described in Bormann, E. R. et al. (1992) Mol. Microbiol. 6: 317-326.

To assess the presence or relative quantity of protein translated from this mRNA, standard techniques, such as a Western blot, may be employed (see, for example, Ausubel et al. (1988) Current Protocols in Molecular Biology, Wiley: N.Y.). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or colorimetric label which may be readily detected. The presence and quantity of label observed indicates the presence and quantity of the desired mutant protein present in the cell.

### EXAMPLE 7

### Growth of Genetically Modified Corynebacterium glutamicum—Media and Culture Conditions

Genetically modified Corynebacteria are cultured in synthetic or natural growth media. A number of different growth media for Corynebacteria are both well-known and readily available (Lieb el al. (1989) Appl. Microbiol. Biolechnol., 32:205-210; von der Osten el al. (1998) Biotechnology Letters, 11:11-16; Patent DE 4,120,867; Liebl (1992) “The Genus Corynebacterium, in: The Procaryotes, Volume II, Balows, A. et al., eds. Springer-Verlag). These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribulose, lactose, maltose, sucrose, raffinose, starch or cellulose serve as very good carbon sources. It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

Inorganic salt compounds which may be included in the media include the chloride-, phosphorous- or sulfate- salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamin, folic acid, nicotinic acid, pantothenate and pyridoxin. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (eds. P. M. Rhodes, P. F. Stanbury, IRL Press (1997) pp.53-73, ISBN 0 19 963577 3). It is also possible to select growth media from commercial suppliers, like standard 1 (Merck) or BHI (grain heart infusion, DIFCO) or others.

All medium components are sterilized, either by heat (20 minutes at 1.5 bar and 121° C.) or by sterile filtration. The components can either be sterilized together or, if necessary, separately. All media components can be present at the beginning of growth, or they can optionally be added continuously or batchwise.

Culture conditions are defined separately for each experiment. The temperature should be in a range between 15° C. and 45° C. The temperature can be kept constant or can be altered during the experiment. The pH of the medium should be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the micro-organisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100-300 rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD₆₀₀ of O.5-1.5 using cells grown on agar plates, such as CM plates (10 g/l glucose, 2.5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/l meat extract, 22 g/l agar, pH 6.8 with 2M NaOH) that had been incubated at 30° C. Inoculation of the media is accomplished by either introduction of a saline suspension of C. glutamicum cells from CM plates or addition of a liquid preculture of this bacterium.

### EXAMPLE 8

### In Vitro Analysis of the Function of Mutant Proteins

The determination of activities and kinetic parameters of enzymes is well established in the art. Experiments to determine the activity of any given altered enzyme must be tailored to the specific activity of the wild-type enzyme, which is well within the ability of one of ordinary skill in the art. Overviews about enzymes in general, as well as specific details concerning structure, kinetics, principles, methods, applications and examples for the determination of many enzyme activities may be found, for example, in the following references: Dixon, M., and Webb, E. C., (1979) Enzymes. Longmans: London; Fersht, (1985) Enzyme Structure and Mechanism. Freeman: N.Y.; Walsh, (1979) Enzymatic Reaction Mechanisms. Freeman: San Francisco; Price, N.C., Stevens, L. (1982) Fundamentals of Enzymology. Oxford Univ. Press: Oxford; Boyer, P. D., ed. (1983) The Enzymes, 3^{rd} ed. Academic Press: New York; Bisswanger, H., (1 994) Enzymkinetik, 2^{nd} ed. VCH: Weinheim (ISBN 3527300325); Bergmeyer, H. U., Bergmeyer, J., Graβl, M., eds. (1983-1986) Methods of Enzymatic Analysis, 3^{rd} ed., vol. I-XII, Verlag Chemie: Weinheim; and Ullmann's Encyclopedia of Industrial Chemistry (1987) vol. A9, “Enzymes”. VCH: Weinheim, p. 352-363.

The activity of proteins which bind to DNA can be measured by several well-established methods, such as DNA band-shift assays (also called gel retardation assays). The effect of such proteins on the expression of other molecules can be measured using reporter gene assays (such as that described in Kolmar, H. et al. (1995) EMBO J 14: 3895-3904 and references cited therein). Reporter gene test systems are well known and established for applications in both pro- and eukaryotic cells, using enzymes such as beta-galactosidase, green fluorescent protein, and several others.

The determination of activity of membrane-transport proteins can be performed according to techniques such as those described in Gennis, R. B. (1989) “Pores, Channels and Transporters”, in Biomembranes, Molecular Structure and Function, Springer: Heidelberg, p. 85-137; 199-234; and 270-322.

### EXAMPLE 9

### Analysis of Impact of Mutant Protein on the Production of the Desired Product

The effect of the genetic modification in C. glutamicum on production of a desired compound (such as an amino acid) can be assessed by growing the modified microorganism under suitable conditions (such as those described above) and analyzing the medium and/or the cellular component for increased production of the desired product (i.e., an amino acid). Such analysis techniques are well known to one of ordinary skill in the art, and include spectroscopy, thin layer chromatography, staining methods of various kinds, enzymatic and microbiological methods, and analytical chromatography such as high performance liquid chromatography (see, for example, Ullman, Encyclopedia of Industrial Chemistry, vol. A2, p. 89-90 and p. 443-613, VCH: Weinheim (1985); Fallon, A. et al., (1987) “Applications of HPLC in Biochemistry” in: Laboratory Techniques in Biochemistry and Molecular Biology, vol. 17; Rehm et al. (1993) Biotechnology, vol. 3, Chapter III: “Product recovery and purification”, page 469-714, VCH: Weinheim; Belter, P. A. el at. (1988) Bioseparations: downstream processing for biotechnology, John Wiley and Sons; Kennedy, J. F. and Cabral, J. M. S. (1992) Recovery processes for biological materials, John Wiley and Sons; Shaeiwitz, J. A. and Henry, J. D. (1988) Biochemical separations, in: Ulmann's Encyclopedia of Industrial Chemistry, vol. B3, Chapter 11, page 1-27, VCH: Weinheim; and Dechow, F. J. (1989) Separation and purification techniques in biotechnology, Noyes Publications.)

In addition to the measurement of the final product of fermentation, it is also possible to analyze other components of the metabolic pathways utilized for the production of the desired compound, such as intermediates and side-products, to determine the overall yield, production, and/or efficiency of production of the compound. Analysis methods include measurements of nutrient levels in the medium (e.g., sugars, hydrocarbons, nitrogen sources, phosphate, and other ions), measurements of biomass composition and growth, analysis of the production of common metabolites of biosynthetic pathways, and measurement of gasses produced during fermentation. Standard methods for these measurements are outlined in Applied Microbial Physiology, A Practical Approach, P. M. Rhodes and P. F. Stanbury, eds., IRL Press, p. 103-129; 131-163; and 165-192 (ISBN: 0199635773) and references cited therein.

### EXAMPLE 10

### Purification of the Desired Product from C. glutamicum Culture

Recovery of the desired product from the C. glutamicum cells or supernatant of the above-described culture can be performed by various methods well known in the art. If the desired product is not secreted from the cells, the cells can be harvested from the culture by low-speed centrifugation, the cells can be lysed by standard techniques, such as mechanical force or sonication. The cellular debris is removed by centrifugation, and the supernatant fraction containing the soluble proteins is retained for further purification of the desired compound. If the product is secreted from the C. glutamicum cells, then the cells are removed from the culture by low-speed centrifugation, and the supernate fraction is retained for further purification.

The supernatant fraction from either purification method is subjected to chromatography with a suitable resin, in which the desired molecule is either retained on a chromatography resin while many of the impurities in the sample are not, or where the impurities are retained by the resin while the sample is not. Such chromatography steps may be repeated as necessary, using the same or different chromatography resins. One of ordinary skill in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There are a wide array of purification methods known to the art and the preceding method of purification is not meant to be limiting. Such purification techniques are described, for example, in Bailey, J. E. & Ollis, D. F. Biochemical Engineering Fundamentals, McGraw-Hill: New York (1986).

The identity and purity of the isolated compounds may be assessed by techniques standard in the art. These include high-performance liquid chromatography (HPLC), spectroscopic methods, staining methods, thin layer chromatography, NIRS, enzymatic assay, or microbiologically. Such analysis methods are reviewed in: Patek et al. (1994) Appl. Environ. Microbiol. 60: 133-140; Malakiova et al. (1996) Biotekhnologiya 11: 27-32; and Schmidt et al. (1998) Bioprocess Engineer. 19: 67-70. Ulmann's Encyclopedia of Industrial Chemistry, (1996) vol. A27, VCH: Weinheim, p. 89-90, p. 521-540, p. 540-547, p. 559-566, 575-581 and p. 581-587; Michal, G. (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, vol. 17.

### EXAMPLE 11

### Analysis of the Gene Sequences of the Invention

The comparison of sequences and determination of percent homology between two sequences are art-known techniques, and can be accomplished using a mathematical algorithm, such as the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to MR nucleic acid molecules of the invention. BLAST protein searches can be performed with the XBLAST program, score=50, wordlength=3 to obtain amino acid sequences homologous to MR protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, one of ordinary skill in the art will know how to optimize the parameters of the program (e.g., XBLAST and NBLAST) for the specific sequence being analyzed.

Another example of a mathematical algorithm utilized for the comparison of sequences is the algorithm of Meyers and Miller ((1988) Comput. Appl. Biosci. 4: 11-17). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art, and include ADVANCE and ADAM described in Torelli and Robotti (1994) Comput. Appli. Biosci. 10:3-5; and FASTA, described in Pearson and Lipman (1988) P.N.A.S. 85:2444-8.

The percent homology between two amino acid sequences can also be accomplished using the GAP program in the GCG software package (available at http://www.gcg.com), using either a Blosum 62 matrix or a PAM250 matrix, and a gap weight of 12, 10, 8, 6, or 4 and a length weight of 2, 3, or 4. The percent homology between two nucleic acid sequences can be accomplished using the GAP program in the GCG software package, using standard parameters, such as a gap weight of 50 and a length weight of 3.

A comparative analysis of the gene sequences of the invention with those present in Genbank has been performed using techniques known in the art (see, e.g., Bexevanis and Ouellette, eds. (1998) Bioinformatics: A Practical Guide to the Analysis of Genes and Proteins. John Wiley and Sons: New York). The gene sequences of the invention were compared to genes present in Genbank in a three-step process. In a first step, a BLASTN analysis (e.g., a local alignment analysis) was performed for each of the sequences of the invention against the nucleotide sequences present in Genbank, and the top 500 hits were retained for further analysis. A subsequent FASTA search (e.g., a combined local and global alignment analysis, in which limited regions of the sequences are aligned) was performed on these 500 hits. Each gene sequence of the invention was subsequently globally aligned to each of the top three FASTA hits, using the GAP program in the GCG software package (using standard parameters). In order to obtain correct results, the length of the sequences extracted from Genbank were adjusted to the length of the query sequences by methods well-known in the art. The results of this analysis are set forth in Table 4. The resulting data is identical to that which would have been obtained had a GAP (global) analysis alone been performed on each of the genes of the invention in comparison with each of the references in Genbank, but required significantly reduced computational time as compared to such a database-wide GAP (global) analysis. Sequences of the invention for which no alignments above the cutoff values were obtained are indicated on Table 4 by the absence of alignment information. It will further be understood by one of ordinary skill in the art that the GAP alignment homology percentages set forth in Table 4 under the heading “% homology (GAP)” are listed in the European numerical format, wherein a ‘,’ represents a decimal point. For example, a value of “40,345” in this column represents “40.345%”.

### EXAMPLE 12

### Construction and Operation of DNA Microarrays

The sequences of the invention may additionally be used in the construction and application of DNA microarrays (the design, methodology, and uses of DNA arrays are well known in the art, and are described, for example, in Schena, M. et al. (1995) Science 270: 467-470; Wodicka, L. et al. (1997) Nature Biotechnology 15: 1359-1367; DeSaizieu, A. et al. (1998) Nature Biotechnology 16: 45-48; and DeRisi, J. L. et al. (1997) Science 278: 680-686).

DNA microarrays are solid or flexible supports consisting of nitrocellulose, nylon, glass, silicone, or other materials. Nucleic acid molecules may be attached to the surface in an ordered manner. After appropriate labeling, other nucleic acids or nucleic acid mixtures can be hybridized to the immobilized nucleic acid molecules, and the label may be used to monitor and measure the individual signal intensities of the hybridized molecules at defined regions. This methodology allows the simultaneous quantification of the relative or absolute amount of all or selected nucleic acids in the applied nucleic acid sample or mixture. DNA microarrays, therefore, permit an analysis of the expression of multiple (as many as 6800 or more) nucleic acids in parallel (see, e.g., Schena, M. (1996) BioEssays 18(5): 427-431).

The sequences of the invention may be used to design oligonucleotide primers which are able to amplify defined regions of one or more C. glutamicum genes by a nucleic acid amplification reaction such as the polymerase chain reaction. The choice and design of the 5′ or 3′ oligonucleotide primers or of appropriate linkers allows the covalent attachment of the resulting PCR products to the surface of a support medium described above (and also described, for example, Schena, M. et al. (1995) Science 270: 467-470).

Nucleic acid microarrays may also be constructed by in situ oligonucleotide synthesis as described by Wodicka, L. et al. (1997) Nature Biotechnology 15: 1359-1367. By photolithographic methods, precisely defined regions of the matrix are exposed to light. Protective groups which are photolabile are thereby activated and undergo nucleotide addition, whereas regions that are masked from light do not undergo any modification. Subsequent cycles of protection and light activation permit the synthesis of different oligonucleotides at defined positions. Small, defined regions of the genes of the invention may be synthesized on microarrays by solid phase oligonucleotide synthesis.

The nucleic acid molecules of the invention present in a sample or mixture of nucleotides may be hybridized to the microarrays. These nucleic acid molecules can be labeled according to standard methods. In brief, nucleic acid molecules (e.g., mRNA molecules or DNA molecules) are labeled by the incorporation of isotopically or fluorescently labeled nucleotides, e.g., during reverse transcription or DNA synthesis. Hybridization of labeled nucleic acids to microarrays is described (e.g., in Schena, M. et al. (1995) supra; Wodicka, L. et al. (1997), szipra; and DeSaizieu A. et al. (1998), supra). The detection and quantification of the hybridized molecule are tailored to the specific incorporated label. Radioactive labels can be detected, for example, as described in Schena, M. et al. (1995) supra) and fluorescent labels may be detected, for example, by the method of Shalon et al. (1996) Genome Research 6: 639-645).

The application of the sequences of the invention to DNA microarray technology, as described above, permits comparative analyses of different strains of C. glutamicum or other Corynebacteria. For example, studies of inter-strain variations based on individual transcript profiles and the identification of genes that are important for specific and/or desired strain properties such as pathogenicity, productivity and stress tolerance are facilitated by nucleic acid array methodologies. Also, comparisons of the profile of expression of genes of the invention during the course of a fermentation reaction are possible using nucleic acid array technology.

### EXAMPLE 13

### Analysis of the Dynamics of Cellular Protein Populations (Proteomics)

The genes, compositions, and methods of the invention may be applied to study the interactions and dynamics of populations of proteins, termed ‘proteomics’. Protein populations of interest include, but are not limited to, the total protein population of C. glutamicum (e.g., in comparison with the protein populations of other organisms), those proteins which are active under specific environmental or metabolic conditions (e.g., during fermentation, at high or low temperature, or at high or low pH), or those proteins which are active during specific phases of growth and development.

Protein populations can be analyzed by various well-known techniques, such as gel electrophoresis. Cellular proteins may be obtained, for example, by lysis or extraction, and may be separated from one another using a variety of electrophoretic techniques. Sodium dodecyl sulfate polyacrylamide gel electrophoresis (SDS-PAGE) separates proteins largely on the basis of their molecular weight. Isoelectric focusing polyacrylamide gel electrophoresis (IEF-PAGE) separates proteins by their isoelectric point (which reflects not only the amino acid sequence but also posttranslational modifications of the protein). Another, more preferred method of protein analysis is the consecutive combination of both IEF-PAGE and SDS-PAGE, known as 2-D-gel electrophoresis (described, for example, in Hermann el al. (1998) Electrophoresis 19: 3217-3221; Fountoulakis et al. (1998) Electrophoresis 19: 1193-1202; Langen et al. (1997) Electrophoresis 18: 1184-1192; Antelmann el al. (1997) Electrophoresis 18: 1451-1463). Other separation techniques may also be utilized for protein separation, such as capillary gel electrophoresis; such techniques are well known in the art.

Proteins separated by these methodologies can be visualized by standard techniques, such as by staining or labeling. Suitable stains are known in the art, and include Coomassie Brilliant Blue, silver stain, or fluorescent dyes such as Sypro Ruby (Molecular Probes). The inclusion of radioactively labeled amino acids or other protein precursors (e.g., ³⁵ S-cysteine, ¹⁴C-labelled amino acids, ¹⁵N-amino acids, ¹⁵NO₃ or ¹⁵NH₄⁺ or ¹³C-labelled amino acids) in the medium of C. glutamicum permits the labeling of proteins from these cells prior to their separation. Similarly, fluorescent labels may be employed. These labeled proteins can be extracted, isolated and separated according to the previously described techniques.

Proteins visualized by these techniques can be further analyzed by measuring the amount of dye or label used. The amount of a given protein can be determined quantitatively using, for example, optical methods and can be compared to the amount of other proteins in the same gel or in other gels. Comparisons of proteins on gels can be made, for example, by optical comparison, by spectroscopy, by image scanning and analysis of gels, or through the use of photographic films and screens. Such techniques are well-known in the art.

To determine the identity of any given protein, direct sequencing or other standard techniques may be employed. For example, N— and/or C-terminal amino acid sequencing (such as Edman degradation) may be used, as may mass spectrometry (in particular MALDI or ESI techniques (see, e.g., Langen et al. (1997) Electrophoresis 18: 1184-1192)). The protein sequences provided herein can be used for the identification of C. glutamicum proteins by these techniques.

The information obtained by these methods can be used to compare patterns of protein presence, activity, or modification between different samples from various biological conditions (e.g., different organisms, time points of fermentation, media conditions, or different biotopes, among others). Data obtained from such experiments alone, or in combination with other techniques, can be used for various applications, such as to compare the behavior of various organisms in a given (e.g., metabolic) situation, to increase the productivity of strains which produce fine chemicals or to increase the efficiency of the production of fine chemicals.

Equivalents

Those of ordinary skill in the art will recognize, or will be able to ascertain using no more than routine experimentation, many equivalents to the specific embodiments of the invention described herein. Such equivalents are intended to be encompassed by the following claims.

TABLE 1
GENES INCLUDED IN THE APPLICATION

| Nucleic | Amino | | | | | |
|---|---|---|---|---|---|---|
| Acid | Acid | Identifi- | | | | |
| SEQ | SEQ | cation | | NT | NT | |
| ID NO | ID NO | Code | Contig. | Start | Stop | Function |
| 1 | 2 | RXN03181 | VV0338 | 196 | 609 | GLUCOSE-RESISTANCE AMYLASE REGULATOR |
| 3 | 4 | F RXA02880 | GR10018 | 417 | 4 | TRANSCRIPTIONAL REPRESSOR CYTR |
| 5 | 6 | RXA00603 | GR00159 | 4982 | 5434 | LEUCINE-RESPONSIVE REGULATORY PROTEIN |
| 7 | 8 | RXN02946 | VV0127 | 7000 | 7458 | FATTY ACYL RESPONSIVE REGULATOR |
| 9 | 10 | RXN01845 | VV0234 | 1093 | 686 | FUMARATE AND NITRATE REDUCTION REGULATORY PROTEIN |
| 11 | 12 | RXN02910 | VV0135 | 30560 | 29856 | TRANSCRIPTIONAL ACTIVATOR PROTEIN LYSR |
| 13 | 14 | RXN02553 | VV0101 | 3454 | 4017 | CRYPTIC BETA-GLUCOSIDE BGL OPERON ANTITERMINATOR |
| 15 | 16 | RXS00686 | VV0005 | 30857 | 30054 | ACETATE OPERON REPRESSOR |
| 17 | 18 | RXS00774 | VV0103 | 22950 | 22297 | PHOSPHATE TRANSPORT SYSTEM REGULATORY PROTEIN |
| 19 | 20 | RXN02493 | VV0007 | 8481 | 9719 | PHOSPHATE REGULON SENSOR PROTEIN PHOR (EC 2.7.3.-) |
| 21 | 22 | F RXA02493 | GR00720 | 2931 | 4169 | regulatory gene for the phosphate regulon |
| 23 | 24 | RXN00631 | VV0135 | 18302 | 16848 | PHOSPHATE REGULON SENSOR PROTEIN PHOR (EC 2.7.3.-) |

Genes for signal transduction pathways, regulation of proteins and transcription

| | | | | | | |
|---|---|---|---|---|---|---|
| 25 | 26 | RXN00291 | VV0041 | 6431 | 4860 | SENSOR KINASE CITA (EC 2.7.3.-) |
| 27 | 28 | F RXA00291 | GR00047 | 2 | 1075 | SENSOR KINASE CITA (EC 2.7.3.-) |
| 29 | 30 | RXA00129 | GR00020 | 6205 | 4709 | SENSOR PROTEIN CPXA (EC 2.7.3.-) |
| 31 | 32 | RXN00651 | VV0109 | 8052 | 9383 | Hypothetical Sensor Histidine Kinase (EC 2.7.3.-) |
| 33 | 34 | F RXA00651 | GR00169 | 5450 | 4119 | SENSOR PROTEIN DEGS (EC 2.7.3.-) |
| 35 | 36 | RXA00006 | GR00001 | 6905 | 6471 | SENSOR PROTEIN FIXL (EC 2.7.3.-) |
| 37 | 38 | RXA01860 | GR00529 | 2368 | 1484 | SENSOR PROTEIN FIXL (EC 2.7.3.-) |
| 39 | 40 | RXA01861 | GR00529 | 4332 | 2368 | SENSOR PROTEIN FIXL (EC 2.7.3.-) |
| 41 | 42 | RXA02669 | GR00753 | 8893 | 10008 | SENSOR PROTEIN RESE (EC 2.7.3.-) |
| 43 | 44 | RXN01211 | VV0169 | 5106 | 6362 | SENSOR PROTEIN UHPB (EC 2.7.3.-) |
| 45 | 46 | F RXA01211 | GR00349 | 741 | 1535 | SENSOR PROTEIN UHPB (EC 2.7.3.-) |
| 47 | 48 | RXA01248 | GR00362 | 165 | 593 | SENSORY TRANSDUCTION PROTEIN REGX3 |
| 49 | 50 | RXA02668 | GR00753 | 8171 | 8893 | SENSORY TRANSDUCTION PROTEIN REGX3 |
| 51 | 52 | RXA02632 | GR00748 | 4863 | 4168 | putative two-component response regulator |
| | | | | | | [Mycobacterium tuberculosis] |
| 53 | 54 | RXA02631 | GR00748 | 4096 | 2732 | putative two-component sensor |
| | | | | | | [Mycobacterium tuberculosis] |
| 55 | 56 | RXA00609 | GR00161 | 226 | 891 | TWO COMPONENT RESPONSE REGULATOR |
| 57 | 58 | RXA00284 | GR00045 | 1318 | 2382 | ANKYRIN HOMOLOG PRECURSOR |
| 59 | 60 | RXA01827 | GR00516 | 6308 | 4902 | PROTEIN KINASE PKNA |
| 61 | 62 | RXA00813 | GR00219 | 1345 | 2475 | SECRETORY PROTEIN KINASE |
| 63 | 64 | RXA01826 | GR00516 | 4902 | 2965 | PUTATIVE SERINE/THREONINE-PROTEIN KINASE |
| | | | | | | PKNB (EC 2.7.1.-) |
| 65 | 66 | RXA02699 | GR00757 | 1357 | 3504 | PUTATIVE SERINE/THREONINE-PROTEIN KINASE |
| | | | | | | PKNB (EC 2.7.1.-) |
| 67 | 68 | RXA00319 | GR00056 | 505 | 80 | LOW MOLECULAR WEIGHT PHOSPHOTYROSINE PROTEIN |
| | | | | | | PHOSPHATASE (EC 3.1.3.48) |
| 69 | 70 | RXA01272 | GR00367 | 25049 | 24447 | PROBABLE LOW MOLECULAR WEIGHT PROTEIN-TYROSINE- |
| | | | | | | PHOSPHATASE EPSP (EC 3.1.3.48) |
| 71 | 72 | RXA01830 | GR00516 | 10410 | 9058 | PUTATIVE PHOSPHOPROTEIN PHOSPHATASE |
| 73 | 74 | RXA02747 | GR00764 | 277 | 2352 | [PROTEIN-PII] URIDYLYLTRANSFERASE |
| | | | | | | (EC 2.7.7.59) |
| 75 | 76 | RXA02210 | GR00648 | 1922 | 2485 | Hypothetical Transcriptional Regulator |
| 77 | 78 | RXA00221 | GR00032 | 20855 | 21073 | Hypothetical Transcriptional Regulator |
| 79 | 80 | RXN00551 | VV0079 | 30941 | 30471 | Hypothetical Transcriptional Regulator |
| 81 | 82 | F RXA00551 | GR00144 | 352 | 5 | Hypothetical Transcriptional Regulator |
| 83 | 84 | RXA01763 | GR00500 | 1987 | 1523 | Hypothetical Transcriptional Regulator |
| 85 | 86 | RXA02667 | GR00753 | 7863 | 7270 | Hypothetical Transcriptional Regulator |
| 87 | 88 | RXA00348 | GR00065 | 1507 | 1052 | Hypothetical Transcriptional Regulator |
| 89 | 90 | RXA01500 | GR00424 | 7551 | 7108 | Hypothetical Transcriptional Regulator |
| 91 | 92 | RXA01125 | GR00312 | 1800 | 1588 | Hypothetical Transcriptional Regulator |
| 93 | 94 | RXN00822 | VV0054 | 21521 | 20841 | Hypothetical Transcriptional Regulator |
| 95 | 96 | F RXA00822 | GR00221 | 3073 | 2393 | putative transcriptional regulator |
| 97 | 98 | RXN00849 | VV0067 | 4701 | 4381 | Hypothetical Transcriptional Regulator |
| 99 | 100 | F RXA00849 | GR00231 | 378 | 698 | possible transcriptional regulator |
| 101 | 102 | RXA02698 | GR00757 | 1143 | 775 | PUTATIVE TRANSCRIPTIONAL REGULATOR |
| 103 | 104 | RXA00350 | GR00066 | 1144 | 1470 | Hypothetical Transcription Inintiation Factor |
| 105 | 106 | RXA02830 | GR00817 | 3 | 497 | Helix-turn-helix domain-containing transcription |
| | | | | | | regulators |
| 107 | 108 | RXA00947 | GR00259 | 4164 | 3829 | Helix-turn-helix domain-containing transcriptional |
| | | | | | | regulators |
| 109 | 110 | RXA01836 | GR00517 | 4370 | 3666 | (AL021287) probable transcriptional regulator |
| | | | | | | [Mycobacterium tuberculosis] |
| 111 | 112 | RXA00292 | GR00047 | 1078 | 1731 | transcriptional regulator CriR |
| 113 | 114 | RXA00182 | GR00028 | 4247 | 7348 | POSSIBLE GLOBAL TRANSCRIPTION ACTIVATOR SNF2L |
| 115 | 116 | RXA02760 | GR00767 | 1154 | 201 | TRANSCRIPTION ANTITERMINATION PROTEIN NUSG |
| 117 | 118 | RXA02306 | GR00663 | 3214 | 2924 | TRANSCRIPTIONAL REGULATORY PROTEIN CITB |
| 119 | 120 | RXA00130 | GR00020 | 6985 | 6308 | TRANSCRIPTIONAL REGULATORY PROTEIN CPXR |
| 121 | 122 | RXA00885 | GR00242 | 11301 | 12326 | HEAT-INDUCIBLE TRANSCRIPTION REPRESSOR HRCA |
| 123 | 124 | RXA01418 | GR00415 | 776 | 531 | TRANSCRIPTIONAL REPRESSOR SMTB |
| 125 | 126 | RXA01759 | GR00498 | 4075 | 4836 | TRANSCRIPTIONAL REGULATORY PROTEIN GLTC |
| 127 | 128 | RXN00363 | VV0176 | 35684 | 34965 | Hypothetical Transcriptional Regulator |
| 129 | 130 | F RXA00363 | GR00073 | 1929 | 1246 | NTA OPERON TRANSCRIPTIONAL REGULATOR |
| 131 | 132 | RXA00516 | GR00131 | 592 | 1311 | NTA OPERON TRANSCRIPTIONAL REGULATOR |
| 133 | 134 | RXA01537 | GR00427 | 4829 | 4179 | NTA OPERON TRANSCRIPTIONAL REGULATOR |
| 135 | 136 | RXA02494 | GR00720 | 4169 | 4864 | KDP OPERON TRANSCRIPTIONAL REGULATORY PROTEIN KDPE |
| 137 | 138 | RXA00029 | GR00003 | 8910 | 8374 | PUTATIVE AGA OPERON TRANSCRIPTIONAL REPRESSOR |
| 139 | 140 | RXA00655 | GR00169 | 9049 | 8411 | putative regulatory protein |
| 141 | 142 | RXN03136 | VV0128 | 2692 | 278 | Hypothetical Transcriptional Regulator |
| 143 | 144 | F RXA00645 | GR00168 | 5831 | 8161 | PUTATIVE REGULATORY PROTEIN |
| 145 | 146 | RXA00593 | GR00158 | 2858 | 2511 | REGULATORY PROTEIN |
| 147 | 148 | RXA02724 | GR00760 | 870 | 4 | REGULATORY PROTEIN |
| 149 | 150 | RXA00494 | GR00123 | 768 | 472 | Hypothetical Regulatory Protein |
| 151 | 152 | RXN01368 | VV0091 | 3096 | 2785 | Hypothetical Regulatory Protein |
| 153 | 154 | F RXA01368 | GR00397 | 2334 | 2206 | Hypothetical Regulatory Protein |
| 155 | 156 | RXN00464 | VV0086 | 61883 | 62656 | REGULATORY PROTEIN SIR2 HOMOLOG |
| 157 | 158 | F RXA00464 | GR00117 | 75 | 332 | REGULATORY PROTEIN SIR2 HOMOLOG |
| 159 | 160 | RXA01655 | GR00460 | 1458 | 100 | PROBABLE RHIZOPINE CATABOLISM REGULATORY |
| | | | | | | PROTEIN MOCR |
| 161 | 162 | RXA00126 | GR00020 | 2269 | 1607 | PROBABLE SIGMA(54) MODULATION PROTEIN |
| 163 | 164 | RXN02450 | VV0107 | 10940 | 10386 | Hypothetical Transcriptional Regulator |
| 165 | 166 | F RXA02450 | GR00710 | 2533 | 3087 | POTENTIAL ACRAB OPERON REPRESSOR |
| 167 | 168 | RXA01898 | GR00544 | 1178 | 1870 | OPERON REGULATOR |
| 169 | 170 | RXA00004 | GR00001 | 4293 | 3823 | NITRILASE REGULATOR |
| 171 | 172 | RXA01001 | GR00284 | 516 | 833 | hex regulon repressor hexR |
| 173 | 174 | RXA01375 | GR00400 | 2560 | 1106 | FRNA |
| 175 | 176 | RXA02831 | GR00818 | 411 | 4 | EXTRAGENIC SUPPRESSOR PROTEIN SUHB |
| 177 | 178 | RXA01110 | GR00306 | 16399 | 16971 | TETRACYCLINE REPRESSOR PROTEIN CLASS C |
| 179 | 180 | RXA00253 | GR00038 | 1064 | 1801 | TETRACYCLINE REPRESSOR PROTEIN CLASS E |
| 181 | 182 | RXA01118 | GR00309 | 1787 | 2551 | regulator of the glyoxylate bypass |
| 183 | 184 | RXA01840 | GR00521 | 2 | 655 | ALIPHATIC AMIDASE EXPRESSION-REGULATING PROTEIN |
| 185 | 186 | RXA00400 | GR00087 | 1163 | 2041 | ALS OPERON REGULATORY PROTEIN |
| 187 | 188 | RXA02787 | GR00777 | 865 | 2241 | ACTIVATOR 1 41 KD SUBUNIT |
| 189 | 190 | RXA00287 | GR00046 | 1618 | 1145 | ADAPTIVE RESPONSE REGULATORY PROTEIN |
| 191 | 192 | RXA01687 | GR00470 | 3289 | 2219 | N-ACETYLGLUCOSAMINE REPRESSOR |
| 193 | 194 | RXA01935 | GR00555 | 8902 | 7739 | N-ACETYLGLUCOSAMINE REPRESSOR |
| 195 | 196 | RXN02270 | VV0020 | 13880 | 13260 | Hypothetical Transcriptional Regulator |
| 197 | 198 | F RXA02270 | GR00655 | 5005 | 4385 | member of the regulatory protein family SIR2 |
| 199 | 200 | RXA01241 | GR00359 | 739 | 1218 | LEXA REPRESSOR (EC 3.4.21.88) |
| 201 | 202 | RXA02127 | GR00637 | 2715 | 2062 | 6 ACTVA REGION GENES OF THE ACTINORHODIN |
| | | | | | | BIOSYNTHETIC GENE CLUSTER |
| 203 | 204 | RXA00583 | GR00156 | 10203 | 9466 | Uncharacterized ACR (translation?) |
| 205 | 206 | RXA00592 | GR00158 | 2121 | 1663 | Uncharacterized ACR (translation initiation |
| | | | | | | regulator?) |
| 207 | 208 | RXA00630 | GR00166 | 2 | 160 | (U67196) DNA-binding response regulator |
| | | | | | | [Thermotoga maritima] |
| 209 | 210 | F RXA00638 | GR00167 | 2862 | 3245 | DNA-binding response regulator |
| 211 | 212 | RXA00894 | GR00244 | 1926 | 799 | GTPASE-ACTIVATING PROTEIN 1 |
| 213 | 214 | RXA01450 | GR00419 | 1237 | 1800 | GTP-BINDING PROTEIN |
| 215 | 216 | RXA01451 | GR00419 | 1760 | 2326 | GTP-BINDING PROTEIN |
| 217 | 218 | RXA02376 | GR00689 | 3064 | 1562 | GTP-BINDING PROTEIN |
| 219 | 220 | RXA01065 | GR00298 | 2 | 583 | GTP-BINDING PROTEIN ERA |
| 221 | 222 | RXA02232 | GR00653 | 5286 | 6812 | GTP-BINDING PROTEIN HFLX |
| 223 | 224 | RXA00848 | GR00230 | 2125 | 1955 | GTP-BINDING PROTEIN LEPA |
| 225 | 226 | F RXA00839 | GR00228 | 372 | 4 | GTP-BINDING PROTEIN LEPA |
| 227 | 228 | F RXA00845 | GR00229 | 907 | 5 | GTP-BINDING PROTEIN LEPA |
| 229 | 230 | RXA02365 | GR00686 | 1568 | 1029 | GTP-BINDING PROTEIN LEPA |
| 231 | 232 | F RXA02392 | GR00696 | 1264 | 5 | GTP-BINDING PROTEIN LEPA |
| 233 | 234 | RXA01573 | GR00438 | 5744 | 3663 | 2′,3′-cyclic-nucleotide 2′- |
| | | | | | | posphodiesterase |
| 235 | 236 | RXN01445 | VV0089 | 14702 | 15694 | Hypothetical Sensor Histidine Kinase (EC 2.7.3.-) |
| 237 | 238 | RXN03143 | VV0139 | 1692 | 2822 | Hypothetical Sensor Histidine Kinase (EC 2.7.3.-) |
| 239 | 240 | RXN03071 | VV0040 | 6 | 344 | Hypothetical Sensor Protein |
| 241 | 242 | RXN03072 | VV0040 | 396 | 830 | Hypothetical Sensor Protein |
| 243 | 244 | RXN01773 | VV0015 | 1128 | 1604 | PROTEIN-TYROSINE PHOSPHATASE (EC 3.1.3.48) |
| 245 | 246 | RXN03090 | VV0054 | 5296 | 4076 | SENSORY COMPONENT OF SENSORY TRANSDUCTION HISTIDINE |
| | | | | | | KINASE (EC 2.7.3.-) |
| 247 | 248 | RXN00617 | VV0054 | 4053 | 3826 | SENSORY COMPONENT OF SENSORY TRANSDUCTION HISTIDINE |
| | | | | | | KINASE (EC 2.7.3.-) |
| 249 | 250 | RXN02990 | VV0073 | 1352 | 1948 | REGULATORY PROTEIN RECX |
| 251 | 252 | RXN03100 | VV0064 | 11866 | 11549 | ALIPHATIC AMIDASE EXPRESSION-REGULATING PROTEIN |
| 253 | 254 | RXN00031 | VV0127 | 54780 | 55181 | PHOSPHOHISTIDINE PHOSPHATASE SIXA (EC 3.1.3.-) |
| 255 | 256 | RXN02758 | VV0084 | 29359 | 28061 | PHOSPHOSERINE PHOSPHATASE (EC 3.1.3.3) |
| 257 | 258 | RXN00978 | VV0149 | 1360 | 1974 | NNRR |
| 259 | 260 | RXN01349 | VV0123 | 1531 | 755 | REGULATORY PROTEIN BETI |
| 261 | 262 | RXN00467 | VV0086 | 60275 | 60943 | IRON REPRESSOR |
| 263 | 264 | RXN02954 | VV0015 | 2693 | 3430 | Hypothetical Transcriptional Regulator |
| 265 | 266 | RXN03023 | VV0003 | 6100 | 5744 | Hypothetical Transcriptional Regulator |
| 267 | 268 | RXN03127 | VV0119 | 8276 | 7557 | Hypothetical Transcriptional Regulator |
| 269 | 270 | RXN03155 | VV0186 | 2 | 1669 | Hypothetical Transcriptional Regulator |
| 271 | 272 | RXN01315 | VV0082 | 13796 | 13146 | Hypothetical Transcription Regulator |
| 273 | 274 | RXN00035 | VV0020 | 24855 | 24499 | Hypothetical Transcriptional Regulator |
| 275 | 276 | RXN00049 | VV0174 | 11833 | 11147 | Hypothetical Transcriptional Regulator |
| 277 | 278 | RXN00486 | VV0086 | 22816 | 23724 | Hypothetical Transcriptional Regulator |
| 279 | 280 | RXN01081 | VV0084 | 33995 | 34744 | Hypothetical Transcriptional Regulator |
| 281 | 282 | RXN01160 | VV0151 | 4187 | 3213 | Hypothetical Transcriptional Regulator |
| 283 | 284 | RXN02097 | VV0298 | 184 | 3555 | Hypothetical Transcriptional Regulator |
| 285 | 286 | RXN02266 | VV0020 | 9528 | 10040 | Hypothetical Transcriptional Regulator |
| 287 | 288 | RXN02362 | VV0051 | 11237 | 7539 | Hypothetical Transcriptional Regulator |
| 289 | 290 | RXN02506 | VV0007 | 25030 | 24149 | Hypothetical Transcriptional Regulator |
| 291 | 292 | RXN02620 | VV0129 | 34206 | 33541 | Hypothetical Transcriptional Regulator |
| 293 | 294 | RXN00826 | VV0180 | 2580 | 3110 | Hypothetical Transcriptional Regulator |
| 295 | 296 | RXS00070 | VV0019 | 32468 | 32899 | FERRIC UPTAKE REGULATION PROTEIN |
| 297 | 298 | RXS00133 | VV0046 | 201 | 1013 | NITRATE/NITRITE RESPONSE REGULATOR PROTEIN NARP |
| 299 | 300 | RXS00144 | VV0134 | 20478 | 21053 | PYRIMIDINE OPERON REGULATORY PROTEIN PYRR |
| 301 | 302 | RXS00205 | VV0096 | 4885 | 3779 | CCPA PROTEIN |
| 303 | 304 | RXS00470 | VV0086 | 27401 | 28669 | NITRATE/NITRITE SENSOR PROTEIN NARX (EC 2.7.3.-) |
| 305 | 306 | RXS00471 | VV0086 | 28715 | 29404 | NITRATE/NITRITE RESPONSE REGULATOR PROTEIN NARL |
| 307 | 308 | RXS00481 | VV0086 | 43354 | 43938 | Hypothetical Protein |
| 309 | 310 | RXS00649 | VV0109 | 10679 | 10224 | Hypothetical Cytosolic Protein |
| 311 | 312 | RXS00650 | VV0109 | 9485 | 10120 | NITRATE/NITRITE RESPONSE REGULATOR PROTEIN NARP |
| 313 | 314 | RXS00657 | VV0109 | 2620 | 3522 | ACR Protein |
| 315 | 316 | RXS00719 | VV0232 | 7281 | 5653 | Hypothetical GTP-Binding Protein |
| 317 | 318 | RXS00738 | VV0254 | 3 | 365 | Hypothetical Cytosolic Protein |
| 319 | 320 | RXS01082 | VV0084 | 35406 | 34747 | IRON REPRESSOR |
| 321 | 322 | RXS01123 | VV0143 | 24824 | 25270 | Hypothetical Protein |
| 323 | 324 | RXS01189 | VV0169 | 6366 | 6974 | NITRATE/NITRITE RESPONSE REGULATOR PROTEIN NARP |
| 325 | 326 | RXS01242 | VV0068 | 17647 | 16871 | GLYCEROL-3-PHOSPHATE REGULON REPRESSOR |
| 327 | 328 | RXS01607 | VV0139 | 2822 | 3451 | NITRATE/NITRITE RESPONSE REGULATOR PROTEIN NARP |
| 329 | 330 | RXS01674 | | | | PROBABLE HYDROGEN PEROXIDE-INDUCIBLE GENES ACTIVATOR |
| 331 | 332 | RXS01872 | VV0248 | 2141 | 2968 | TRANSCRIPTIONAL REGULATORY PROTEIN |
| 333 | 334 | RXS02117 | VV0102 | 8076 | 8549 | Hypothetical Cytosolic Protein |
| 335 | 336 | RXS02288 | VV0127 | 51473 | 50628 | GLYCEROL-3-PHOSPHATE REGULON REPRESSOR |
| 337 | 338 | RXS02573 | VV0098 | 2475 | 2918 | ACR Protein |
| 339 | 340 | RXS02627 | VV0314 | 2981 | 2139 | DTXR/IRON-REGULATED LIPOPROTEIN PRECURSOR |
| 341 | 342 | RXS02691 | VV0098 | 55962 | 56768 | FATTY ACYL RESPONSIVE REGULATOR |
| 343 | 344 | RXS02730 | VV0145 | 7640 | 8677 | RIBOSE OPERON REPRESSOR |
| 345 | 346 | RXS02818 | VV0347 | 611 | 6 | Hypothetical Protein |
| 347 | 348 | RXS02911 | VV0135 | 24643 | 25101 | Hypothetical Cytosolic Protein |
| 349 | 350 | RXS03066 | VV0038 | 7298 | 6636 | Hypothetical Protein |
| 351 | 352 | RXS03208 | | | | DIPHTHERIA TOXIN REPRESSOR |
| 353 | 354 | F RXA00307 | GR00052 | 467 | 6 | DIPHTHERIA TOXIN REPRESSOR |
| 355 | 356 | RXS03219 | | | | LACI-FAMILY TRANSCRIPTION REGULATOR |
| 357 | 358 | F RXA02763 | GR00768 | 1603 | 2586 | MALTOSE OPERON TRANSCRIPTIONAL REPRESSOR |
| 359 | 360 | RXS03200 | | | | PROBABLE HYDROGEN PEROXIDE-INDUCIBLE GENES ACTIVATOR |

TABLE 2
GENES IDENTIFIED FROM GENBANK

| GenBank ™ | | | |
|---|---|---|---|
| Accession No. | Gene Name | Gene Function | Reference |
| A09073 | ppg | Phosphoenol pyruvate | Bachmann, B. et al. “DNA fragment coding |
| | | carboxylase | for phosphoenolpyruvat corboxylase, |
| | | | recombinant DNA carrying said fragment, |
| | | | strains carrying the recombinant DNA and |
| | | | method for producing L-amino acids using said |
| | | | strains,” Patent: EP 0358940-A 3 Mar. 21, 1990 |
| A45579, | | Threonine dehydratase | Moeckel, B. et al. “Production of L-isoleucine |
| A45581, | | | by means of recombinant micro-organisms with |
| A45583, | | | deregulated threonine dehydratase,” Patent: |
| A45585 | | | WO 9519442-A 5 Jul. 20, 1995 |
| A45587 | | | |
| AB003132 | murC; ftsQ; | | Kobayashi, M. et al. “Cloning, sequencing, |
| | ftsZ | | and characterization of the ftsZ gene from |
| | | | coryneform bacteria,” Biochem. Biophys. Res. |
| | | | Commun., 236(2): 383-388 (1997) |
| AB015023 | murC; ftsQ | | Wachi, M. et al. “A murC gene from Coryneform |
| | | | bacteria,” Appl. Microbiol. Biotechnol., |
| | | | 51(2): 223-228 (1999) |
| AB018530 | dtsR | | Kimura, E. et al. “Molecular cloning of a |
| | | | novel gene, dtsR, which rescues the |
| | | | detergent sensitivity of a mutant derived from |
| | | | Brevibacterium lactofermentum,” Biosci. |
| | | | Biotechnol. Biochem., 60(10): 1565-1570 (1996) |
| AB018531 | dtsR1; dtsR2 | | |
| AB020624 | murI | D-glutamate racemase | |
| AB023377 | tkt | transketolase | |
| AB024708 | gltB; gltD | Glutamine 2-oxoglutarate | |
| | | aminotransferase | |
| | | large and small subunits | |
| AB025424 | acn | aconitase | |
| AB027714 | rep | Replication protein | |
| AB027715 | rep; aad | Replication protein; | |
| | | aminoglycoside | |
| | | adenyltransferase | |
| AF005242 | argC | N-acetylglutamate-5- | |
| | | semialdehyde | |
| | | dehydrogenase | |
| AF005635 | glnA | Glutamine synthetase | |
| AF030405 | hisF | cyclase | |
| AF030520 | argG | Argininosuccinate synthetase | |
| AF031518 | argF | Ornithine carbamolytransferase | |
| AF036932 | aroD | 3-dehydroquinate dehydratase | |
| AF038548 | pyc | Pyruvate carboxylase | |
| AF038651 | dciAE; apt; | Dipeptide-binding protein; | Wehmeier, L. et al. “The role of the |
| | rel | adenine | Corynebacterium glutamicum rel gene in (p)ppGpp |
| | | phosphoribosyltransferase; | metabolism,” Microbiology, 144: 1853-1862 (1998) |
| | | GTP pyrophosphokinase | |
| AF041436 | argR | Arginine repressor | |
| AF045998 | impA | Inositol monophosphate | |
| | | phosphatase | |
| AF048764 | argH | Argininosuccinate lyase | |
| AF049897 | argC; argJ; | N-acetylglutamylphosphate | |
| | argB; argD; | reductase; ornithine | |
| | argF; argR; | acetyltransferase; N- | |
| | argG; argH | acetylglutamate kinase; | |
| | | acetylornithine | |
| | | transminase; ornithine | |
| | | carbamoyltransferase; | |
| | | arginine repressor; | |
| | | argininosuccinate synthase; | |
| | | argininosuccinate lyase | |
| AF050109 | inhA | Enoyl-acyl carrier protein | |
| | | reductase | |
| AF050166 | hisG | ATP phosphoribosyltransferase | |
| AF051846 | hisA | Phosphoribosylformimino-5- | |
| | | amino-1-phosphoribosyl-4- | |
| | | imidazolecarboxamide | |
| | | isomerase | |
| AF052652 | metA | Homoserine O-acetyltransferase | Park, S. et al. “Isolation and analysis of |
| | | | metA, a methionine biosynthetic gene encoding |
| | | | homoserine acetyltransferase in Corynebacterium |
| | | | glutamicum,” Mol. Cells., 8(3): 286-294 (1998) |
| AF053071 | aroB | Dehydroquinate synthetase | |
| AF060558 | hisH | Glutamine amidotransferase | |
| AF086704 | hisE | Phosphoribosyl-ATP- | |
| | | pyrophosphohydrolase | |
| AF114233 | aroA | 5-enolpyruvylshikimate | |
| | | 3-phosphate synthase | |
| AF116184 | panD | L-aspartate-alpha- | Dusch, N. et al. “Expression of the |
| | | decarboxylase precursor | Corynebacterium glutamicum panD gene encoding |
| | | | L-aspartate-alpha-decarboxylase leads to |
| | | | pantothenate overproduction in Escherichia coli,” |
| | | | Appl. Environ. Microbiol., 65(4)1530-1539 (1999) |
| AF124518 | aroD; aroE | 3-dehydroquinase; shikimate | |
| | | dehydrogenase | |
| AF124600 | aroC; aroK; | Chorismate synthase; shikimate kinase; 3- | |
| | aroB; pepQ | dehydroquinate synthase; | |
| | | putative cytoplasmic peptidase | |
| AF145897 | inhA | | |
| AF145898 | inhA | | |
| AJ001436 | ectP | Transport of ectoine, | Peter, H. et al. “Corynebacterium glutamicum |
| | | glycine betaine, proline | is equipped with four secondary carriers for |
| | | | compatible solutes: Identification, sequencing, |
| | | | and characterization of the proline/ectoine |
| | | | uptake system, ProP, and the ectoine/proline/ |
| | | | glycine betaine carrier, EctP,” J. Bacteriol., |
| | | | 180(22): 6005-6012 (1998) |
| AJ004934 | dapD | Tetrahydrodipicolinate | Wehrmann, A. et al. “Different modes of |
| | | succinylase (incomplete¹) | diaminopimelate synthesis and their role in cell |
| | | | wall integrity: A study with Corynebacterium |
| | | | glutamicum,” J. Bacteriol., 180(12): 3159-3165 (1998) |
| AJ007732 | ppc; secG; | Phosphoenolpyruvate-carboxylase; ?; high | |
| | amt; ocd; | affinity ammonium uptake protein; | |
| | soxA | putative ornithine- | |
| | | cyclodecarboxylase; sarcosine | |
| | | oxidase | |
| AJ010319 | ftsY, glnB, | Involved in cell division; | Jakoby, M. et al. “Nitrogen regulation in |
| | glnD; srp; | PII protein; uridylyltransferase | Corynebacterium glutamicum; Isolation of genes |
| | amtP | (uridylyl-removing enzmye); | involved in biochemical characterization of |
| | | signal recognition particle; | corresponding proteins,” FEMS Microbiol., |
| | | low affinity ammonium uptake | 173(2): 303-310 (1999) |
| | | protein | |
| AJ132968 | cat | Chloramphenicol aceteyl | |
| | | transferase | |
| AJ224946 | mqo | L-malate: quinone oxidoreductase | Molenaar, D. et al. “Biochemical and genetic |
| | | | characterization of the membrane-associated |
| | | | malate dehydrogenase (acceptor) from |
| | | | Corynebacterium glutamicum,” Eur. J. |
| | | | Biochem., 254(2): 395-403 (1998) |
| AJ238250 | ndh | NADH dehydrogenase | |
| AJ238703 | porA | Porin | Lichtinger, T. et al. “Biochemical and |
| | | | biophysical characterization of the cell |
| | | | wall porin of Corynebacterium glutamicum: |
| | | | The channel is formed by a low molecular mass |
| | | | polypeptide,” Biochemistry, 37(43): 15024-15032 (1998) |
| D17429 | | Transposable element IS31831 | Vertes, A. A. et al. “Isolation and |
| | | | characterization of IS31831, a transposable |
| | | | element from Corynebacterium glutamicum,” |
| | | | Mol. Microbiol., 11(4): 739-746 (1994) |
| D84102 | odhA | 2-oxoglutarate dehydrogenase | Usuda, Y. et al. “Molecular cloning of the |
| | | | Corynebacterium glutamicum (Brevibacterium |
| | | | lactofermentum AJ12036) odhA gene encoding a |
| | | | novel type of 2-oxoglutarate dehydrogenase,” |
| | | | Microbiology, 142: 3347-3354 (1996) |
| E01358 | hdh; hk | Homoserine dehydrogenase; | Katsumata, R. et al. “Production of |
| | | homoserine kinase | L-thereonine and L-isoleucine,” Patent: JP |
| | | | 1987232392-A 1 Oct. 12, 1987 |
| E01359 | | Upstream of the start codon | Katsumata, R. et al. “Production of |
| | | of homoserine kinase gene | L-thereonine and L-isoleucine,” Patent: JP |
| | | | 1987232392-A 2 Oct. 12, 1987 |
| E01375 | | Tryptophan operon | |
| E01376 | trpL; trpE | Leader peptide; anthranilate | Matsui, K. et al. “Tryptophan operon, |
| | | synthase | peptide and protein coded thereby, utilization |
| | | | of tryptophan operon gene expression and |
| | | | production of tryptophan,” Patent: |
| | | | JP 1987244382-A 1 Oct. 24, 1987 |
| E01377 | | Promoter and operator regions | Matsui, K. et al. “Tryptophan operon, |
| | | of tryptophan operon | peptide and protein coded thereby, utilization |
| | | | of tryptophan operon gene expression and |
| | | | production of tryptophan,” Patent: |
| | | | JP 1987244382-A 1 Oct. 24, 1987 |
| E03937 | | Biotin-synthase | Hatakeyama, K. et al. “DNA fragment |
| | | | containing gene capable of coding biotin |
| | | | synthetase and its utilization,” Patent: |
| | | | JP 1992278088-A 1 Oct. 02, 1992 |
| E04040 | | Diamino pelargonic acid | Kohama, K. et al. “Gene coding |
| | | aminotransferase | diaminopelargonic acid aminotransferase and |
| | | | desthiobiotin synthetase and its utilization,” |
| | | | Patent: JP 1992330284-A 1 Nov. 18, 1992 |
| E04041 | | Desthiobiotinsynthetase | Kohama, K. et al. “Gene coding |
| | | | diaminopelargonic acid aminotransferase and |
| | | | desthiobiotin synthetase and its utilization,” |
| | | | Patent: JP 1992330284-A 1 Nov. 18, 1992 |
| E04307 | | Flavum aspartase | Kurusu, Y. et al. “Gene DNA coding aspartase |
| | | | and utilization thereof,” Patent: |
| | | | JP 1993030977-A 1 Feb. 09, 1993 |
| E04376 | | Isocitric acid lyase | Katsumata, R. et al. “Gene manifestation |
| | | | controlling DNA,” Patent: |
| | | | JP 1993056782-A 3 Mar. 09, 1993 |
| E04377 | | Isocitric acid lyase N-terminal | Katsumata, R. et al. “Gene manifestation |
| | | fragment | controlling DNA,” Patent: |
| | | | JP 1993056782-A 3 Mar. 09, 1993 |
| E04484 | | Prephenate dehydratase | Sotouchi, N. et al. “Production of |
| | | | L-phenylalanine by fermentation,” Patent: |
| | | | JP 1993076352-A 2 Mar. 30, 1993 |
| E05108 | | Aspartokinase | Fugono, N. et al. “Gene DNA coding |
| | | | Aspartokinase and its use,” Patent: JP |
| | | | 1993184366-A 1 Jul. 27, 1993 |
| E05112 | | Dihydro-dipichorinate synthetase | Hatakeyama, K. et al. “Gene DNA coding |
| | | | dihydrodipicolinic acid synthetase and its use,” |
| | | | Patent: JP 1993184371-A 1 Jul. 27, 1993 |
| E05776 | | Diaminopimelic acid dehydrogenase | Kobayashi, M. et al. “Gene DNA coding |
| | | | Diaminopimelic acid dehydrogenase and its use,” |
| | | | Patent: JP 1993284970-A 1 Nov. 02, 1993 |
| E05779 | | Threonine synthase | Kohama, K. et al. “Gene DNA coding threonine |
| | | | synthase and its use,” Patent: |
| | | | JP 1993284972-A 1 Nov. 02, 1993 |
| E06110 | | Prephenate dehydratase | Kikuchi, T. et al. “Production of |
| | | | L-phenylalanine by fermentation method,” |
| | | | Patent: JP 1993344881-A 1 Dec. 27, 1993 |
| E06111 | | Mutated Prephenate dehydratase | Kikuchi, T. et al. “Production of |
| | | | L-phenylalanine by fermentation method,” |
| | | | Patent: JP 1993344881-A 1 Dec. 27, 1993 |
| E06146 | | Acetohydroxy acid synthetase | Inui, M. et al. “Gene capable of coding |
| | | | Acetohydroxy acid synthetase and its |
| | | | use,” Patent: JP 1993344893-A 1 Dec. 27, 1993 |
| E06825 | | Aspartokinase | Sugimoto, M. et al. “Mutant aspartokinase |
| | | | gene,” patent: JP 1994062866-A 1 Mar. 08, 1994 |
| E06826 | | Mutated aspartokinase alpha | Sugimoto, M. et al. “Mutant aspartokinase |
| | | subunit | gene,” patent: JP 1994062866-A 1 Mar. 08, 1994 |
| E06827 | | Mutated aspartokinase alpha | Sugimoto, M. et al. “Mutant aspartokinase |
| | | subunit | gene,” patent: JP 1994062866-A 1 Mar. 08, 1994 |
| E07701 | secY | | Honno, N. et al. “Gene DNA participating in |
| | | | integration of membraneous protein to membrane,” |
| | | | Patent: JP 1994169780-A 1 Jun. 21, 1994 |
| E08177 | | Aspartokinase | Sato, Y. et al. “Genetic DNA capable of coding |
| | | | Aspartokinase released from feedback inhibition |
| | | | and its utilization,” Patent: |
| | | | JP 1994261766-A 1 Sep. 20, 1994 |
| E08178, | | Feedback inhibition-released | Sato, Y. et al. “Genetic DNA capable of coding |
| E08179, | | Aspartokinase | Aspartokinase released from feedback inhibition |
| E08180, | | | and its utilization,” Patent: |
| E08181, | | | JP 1994261766-A 1 Sep. 20, 1994 |
| E08182 | | | |
| E08232 | | Acetohydroxy-acid | Inui, M. et al. “Gene DNA coding acetohydroxy |
| | | isomeroreductase | acid isomeroreductase,” |
| | | | Patent: JP 1994277067-A 1 Oct. 04, 1994 |
| E08234 | secE | | Asai, Y. et al. “Gene DNA coding for |
| | | | translocation machinery of protein,” |
| | | | Patent: JP 1994277073-A 1 Oct. 04, 1994 |
| E08643 | | FT aminotransferase and | Hatakeyama, K. et al. “DNA fragment having |
| | | desthiobiotin | promoter function in coryneform bacterium,” |
| | | synthetase promoter region | Patent: JP 1995031476-A 1 Feb. 03, 1995 |
| E08646 | | Biotin synthetase | Hatakeyama, K. et al. “DNA fragment having |
| | | | promoter function in coryneform bacterium,” |
| | | | Patent: JP 1995031476-A 1 Feb. 03, 1995 |
| E08649 | | Aspartase | Kohama, K. et al “DNA fragment having promoter |
| | | | function in coryneform bacterium,” Patent: |
| | | | JP 1995031478-A 1 Feb. 03, 1995 |
| E08900 | | Dihydrodipicolinate reductase | Madori, M. et al. “DNA fragment containing |
| | | | gene coding Dihydrodipicolinate acid reductase |
| | | | and utilization thereof,” Patent: |
| | | | JP 1995075578-A 1 Mar. 20, 1995 |
| E08901 | | Diaminopimelic acid decarboxylase | Madori, M. et al. “DNA fragment containing |
| | | | gene coding Diaminopimelic acid decarboxylase |
| | | | and utilization thereof,” Patent: |
| | | | JP 1995075579-A 1 Mar. 20, 1995 |
| E12594 | | Serine hydroxymethyltransferase | Hatakeyama, K. et al. “Production of |
| | | | L-trypophan,” Patent: JP 1997028391-A |
| | | | 1 Feb. 04, 1997 |
| E12760, | | transposase | Moriya, M. et al. “Amplification of gene |
| E12759, | | | using artificial transposon,” Patent: |
| E12758 | | | JP 1997070291-A Mar. 18, 1997 |
| E12764 | | Arginyl-tRNA synthetase; | Moriya, M. et al. “Amplification of gene |
| | | diaminopimelic | using artificial transposon,” Patent: |
| | | acid decarboxylase | JP 1997070291-A Mar. 18, 1997 |
| E12767 | | Dihydrodipicolinic acid | Moriya, M. et al. “Amplification of gene |
| | | synthetase | using artificial transposon,” Patent: |
| | | | JP 1997070291-A Mar. 18, 1997 |
| E12770 | | aspartokinase | Moriya, M. et al. “Amplification of gene |
| | | | using artificial transposon,” Patent: |
| | | | JP 1997070291-A Mar. 18, 1997 |
| E12773 | | Dihydrodipicolinic acid reductase | Moriya, M. et al. “Amplification of gene |
| | | | using artificial transposon,” Patent: |
| | | | JP 1997070291-A Mar. 18, 1997 |
| E13655 | | Glucose-6-phosphate dehydrogenase | Hatakeyama, K. et al. “Glucose-6-phosphate |
| | | | dehydrogenase and DNA capable of coding the |
| | | | same,” Patent: JP 1997224661-A 1 Sep. 02, 1997 |
| L01508 | IlvA | Threonine dehydratase | Moeckel, B. et al. “Functional and structural |
| | | | analysis of the threonine dehydratase of |
| | | | Corynebacterium glutamicum,” J. Bacteriol., |
| | | | 174: 8065-8072 (1992) |
| L07603 | EC 4.2.1.15 | 3-deoxy-D-arabinoheptulosonate-7- | Chen, C. et al. “The cloning and nucleotide |
| | | phosphate synthase | sequence of Corynebacterium glutamicum 3-deoxy-D- |
| | | | arabinoheptulosonate-7-phosphate synthase gene,” |
| | | | FEMS Microbiol. Lett., 107: 223-230 (1993) |
| L09232 | IlvB; ilvN; | Acetohydroxy acid synthase large subunit; | Keilhauer, C. et al. “Isoleucine synthesis in |
| | ilvC | Acetohydroxy acid synthase small subunit; | Corynebacterium glutamicum: molecular analysis of |
| | | Acetohydroxy acid | the ilvB-ilvN-ilvC operon,” J. Bacteriol., |
| | | isomeroreductase | 175(17): 5595-5603 (1993) |
| L18874 | PtsM | Phosphoenolpyruvate sugar | Fouet, A et al. “Bacillus subtilis sucrose- |
| | | phosphotransferase | specific enzyme II of the phosphotransferase |
| | | | system: expression in Escherichia coli and |
| | | | homology to enzymes II from enteric bacteria,” |
| | | | PNAS USA, 84(24): 8773-8777 (1987); Lee, J. K. et al. |
| | | | “Nucleotide sequence of the gene encoding the |
| | | | Corynebacterium glutamicum mannose enzyme II and |
| | | | analyses of the deduced protein sequence,” |
| | | | FEMS Microbiol. Lett., 119(1-2): 137-145 (1994) |
| L27123 | aceB | Malate synthase | Lee, H-S. et al. “Molecular characterization |
| | | | of aceB, a gene encoding malate synthase in |
| | | | Corynebacterium glutamicum,” J. Microbiol. |
| | | | Biotechnol., 4(4): 256-263 (1994) |
| L27126 | | Pyruvate kinase | Jetten, M. S. et al. “Structural and |
| | | | functional analysis of pyruvate kinase from |
| | | | Corynebacterium glutamicum,” Appl. Environ. |
| | | | Microbiol., 60(7): 2501-2507 (1994) |
| L28760 | aceA | Isocitrate lyase | |
| L35906 | dtxr | Diphtheria toxin repressor | Oguiza, J. A. et al. “Molecular cloning, DNA |
| | | | sequence analysis, and characterization of the |
| | | | Corynebacterium diphtheriae dtxR from Brevibacterium |
| | | | lactofermentum,” J. Bacteriol., 177(2): |
| | | | 465-467 (1995) |
| M13774 | | Prephenate dehydratase | Follettie, M. T. et al. “Molecular cloning and |
| | | | nucleotide sequence of the Corynebacterium |
| | | | glutamicum pheA gene,” J. Bacteriol., |
| | | | 167: 695-702 (1986) |
| M16175 | 5S rRNA | | Park, Y-H. et al. “Phylogenetic analysis of |
| | | | the coryneform bacteria by 56 rRNA sequences,” |
| | | | J. Bacteriol., 169: 1801-1806 (1987) |
| M16663 | trpE | Anthranilate synthase, 5′ end | Sano, K. et al. “Structure and function of the |
| | | | trp operon control regions of Brevibacterium |
| | | | lactofermentum, a glutamic-acid-producing |
| | | | bacterium,” Gene, 52: 191-200 (1987) |
| M16664 | trpA | Tryptophan synthase, 3′end | Sano, K. et al. “Structure and function of the |
| | | | trp operon control regions of Brevibacterium |
| | | | lactofermentum, a glutamic-acid-producing |
| | | | bacterium,” Gene, 52: 191-200 (1987) |
| M25819 | | Phosphoenolpyruvate carboxylase | O'Regan, M. et al. “Cloning and nucleotide |
| | | | sequence of the Phosphoenolpyruvate carboxylase- |
| | | | coding gene of Corynebacterium glutamicum |
| | | | ATCC13032,” Gene, 77(2): 237-251 (1989) |
| M85106 | | 23S rRNA gene insertion sequence | Roller, C. et al. “Gram-positive bacteria with |
| | | | a high DNA G + C content are characterized by a |
| | | | common insertion within their 23S rRNA genes,” |
| | | | J. Gen. Microbiol., 138: 1167-1175 (1992) |
| M85107, | | 23S rRNA gene insertion sequence | Roller, C. et al. “Gram-positive bacteria with |
| M85108 | | | a high DNA G + C content are characterized by a |
| | | | common insertion within their 23S rRNA genes,” |
| | | | J. Gen. Microbiol., 138: 1167-1175 (1992) |
| M89931 | aecD; brnQ; | Beta C—S lyase; branched-chain | Rossol, I. et al. “The Corynebacterium |
| | yhbw | amino acid uptake carrier; | glutamicum aecD gene encodes a C—S lyase with |
| | | hypothetical protein yhbw | alpha, beta-elimination activity that degrades |
| | | | aminoethylcysteine,” J. Bacteriol., 174(9): |
| | | | 2968-2977 (1992); Tauch, A. et al. “Isoleucine |
| | | | uptake in Corynebacterium glutamicum ATCC 13032 |
| | | | is directed by the brnQ gene product,”Arch. |
| | | | Microbiol, 169(4): 303-312 (1998) |
| S59299 | trp | Leader gene (promoter) | Herry, D. M. et al. “Cloning of the trp gene |
| | | | cluster from a tryptophan-hyperproducing strain |
| | | | of Corynebacterium glutamicum: identification of a |
| | | | mutation in the trp leader sequence,” Appl. |
| | | | Environ. Microbiol., 59(3): 791-799 (1993) |
| U11545 | trpD | Anthranilate | O'Gara, J. P. and Dunican, L. K. (1994) Complete |
| | | phosphoribosyltransferase | nucleotide sequence of the Corynebacterium |
| | | | glutamicum ATCC 21850 tpD gene.” Thesis, |
| | | | Microbiology Department, University College |
| | | | Galway, Ireland. |
| U13922 | cglIM; cglIR; | Putative type II 5-cytosoine | Schafer, A. et al. “Cloning and characterization |
| | clgIIR | methyltransferase; putative type II | of a DNA region encoding a stress-sensitive |
| | | restriction endonuclease; | restriction system from Corynebacterium glutamicum |
| | | putative type I or type III | ATCC 13032 and analysis of its role in intergeneric |
| | | restriction endonuclease | conjugation with Escherichia coli,” J. |
| | | | Bacteriol., 176(23): 7309-7319 (1994); Schafer, A. |
| | | | et al. “The Corynebacterium glutamicum cglIM gene |
| | | | encoding a 5-cytosine in an McrBC-deficient |
| | | | Escherichia coli strain,” Gene, 203(2): 95-101 (1997) |
| U14965 | recA | | |
| U31224 | ppx | | Ankri, S. et al. “Mutations in the |
| | | | Corynebacterium glutamicum proline biosynthetic |
| | | | pathway: A natural bypass of the proA step,” |
| | | | J. Bacteriol, 178(15): 4412-4419 (1996) |
| U31225 | proC | L-proline: NADP+ | Ankri, S. et al. “Mutations in the |
| | | 5-oxidoreductase | Corynebacterium glutamicum proline biosynthetic |
| | | | pathway: A natural bypass of the proA step,” |
| | | | J. Bacteriol, 178(15): 4412-4419 (1996) |
| U31230 | obg; proB; | ?; gamma glutamyl kinase; | Ankri, S. et al. “Mutations in the |
| | unkdh | similar to D-isomer specific | Corynebacterium glutamicum proline |
| | | 2-hydroxyacid dehydrogenases | biosynthetic pathway: A natural bypass of the |
| | | | proA step,” J. Bacteriol, 178(15): 4412-4419 (1996) |
| U31281 | bioB | Biotin synthase | Serebriiskii, I. G., “Two new members of the |
| | | | bio B superfamily: Cloning, sequencing and |
| | | | expression of bio B genes of Methylobacillus |
| | | | flagellatum and Corynebacterium glutamicum,” |
| | | | Gene, 175: 15-22 (1996) |
| U35023 | thtR; accBC | Thiosulfate sulfurtransferase; | Jager, W. et al. “A Corynebacterium glutamicum |
| | | acyl CoA carboxylase | gene encoding a two-domain protein similar to |
| | | | biotin carboxylases and biotin-carboxyl-carrier |
| | | | proteins,“ Arch. Microbiol., 166(2); 76-82 (1996) |
| U43535 | cmr | Multidrug resistance protein | Jager, W. et al. “A Corynebacterium glutamicum |
| | | | gene conferring multidrug resistance in the |
| | | | heterologous host Escherichia coli,” J. |
| | | | Bacteriol., 179(7): 2449-2451 (1997) |
| U43536 | clpB | Heat shock ATP-binding protein | |
| U53587 | aphA-3 | 3′5″-aminoglycoside | |
| | | phosphotransferase | |
| U89648 | | Corynebacterium glutamicum | |
| | | unidentified sequence involved | |
| | | in histidine biosynthesis, | |
| | | partial sequence | |
| X04960 | trpA; trpB; | Tryptophan operon | Matsui, K. et al. “Complete nucleotide and |
| | trpC; trpD; | | deduced amino acid sequences of the Brevibacterium |
| | trpE; trpG; | | lactofermentum tryptophan operon,” Nucleic |
| | trpL | | Acids Res., 14(24): 10113-10114 (1986) |
| X07563 | lys A | DAP decarboxylase (meso- | Yeh, P. et al. “Nucleic sequence of the lysA |
| | | diaminopimelate | gene of Corynebacterium glutamicum and possible |
| | | decarboxylase, EC 4.1.1.20) | mechanisms for modulation of its expression,” |
| | | | Mol. Gen. Genet., 212(1): 112-119 (1988) |
| X14234 | EC 4.1.1.31 | Phosphoenolpyruvate carboxylase | Eikmanns, B. J. et al. “The Phosphoenolpyruvate |
| | | | carboxylase gene of Corynebacterium glutamicum: |
| | | | Molecular cloning, nucleotide sequence, and |
| | | | expression,” Mol. Gen. Genet., 218(2): 330-339 |
| | | | (1989); Lepiniec, L. et al. “Sorghum |
| | | | Phosphoenolpyruvate carboxylase gene family: |
| | | | structure, function and molecular evolution,” |
| | | | Plant. Mol. Biol., 21 (3): 487-502 (1993) |
| X17313 | fda | Fructose-bisphosphate aldolase | Von der Osten, C. H. et al. “Molecular cloning, |
| | | | nucleotide sequence and fine-structural analysis |
| | | | of the Corynebacterium glutamicum fda gene: |
| | | | structural comparison of C. glutamicum fructose- |
| | | | 1,6-biphosphate aldolase to class I and |
| | | | class II aldolases,” Mol. Microbiol., |
| X53993 | dapA | L-2,3-dihydrodipicolinate | Bonnassie, S. et al. “Nucleic sequence of the |
| | | synthetase (EC 4.2.1.52) | dapA gene from Corynebacterium glutamicum,” |
| | | | Nucleic Acids Res., 18(21): 6421 (1990) |
| X54223 | | AttB-related site | Cianciotto, N. et al. “DNA sequence homology |
| | | | between att B-related sites of Corynebacterium |
| | | | diphtheriae, Corynebacterium ulcerans, |
| | | | Corynebacterium glutamicum, and the attP site |
| | | | of lambdacorynephage,” FEMS. Microbiol, |
| | | | Lett., 66: 299-302 (1990) |
| X54740 | argS; lysA | Arginyl-tRNA synthetase; | Marcel, T. et al. “Nucleotide sequence and |
| | | Diaminopimelate decarboxylase | organization of the upstream region |
| | | | of the Corynebacterium glutamicum lysA gene,” |
| | | | Mol. Microbiol., 4(11): 1819-1830 (1990) |
| X55994 | trpL; trpE | Putative leader peptide; | Heery, D. M. et al. “Nucleotide sequence of |
| | | anthranilate synthase component 1 | the Corynebacterium glutamicum trpE gene,” |
| | | | Nucleic Acids Res., 18(23): 7138 (1990) |
| X56037 | thrC | Threonine synthase | Han, K. S. et al. “The molecular structure of |
| | | | the Corynebacterium glutamicum threonine synthase |
| | | | gene,” Mol. Microbiol., 4(10): 1693-1702 (1990) |
| X56075 | attB-related | Attachment site | Cianciotto, N. et al. “DNA sequence homology |
| | site | | between att B-related sites of Corynebacterium |
| | | | diphtheriae, Corynebacterium ulcerans, |
| | | | Corynebacterium glutamicum, and the attP site of |
| | | | lambdacorynephage,” FEMS. Microbiol, |
| | | | Lett., 66: 299-302 (1990) |
| X57226 | lysC-alpha; | Aspartokinase-alpha subunit; | Kalinowski, J. et al. “Genetic and biochemical |
| | lysC-beta; | Aspartokinase-beta subunit; | analysis of the Aspartokinase from Corynebacterium |
| | asd | aspartate beta | glutamicum,” Mol. Microbiol., 5(5): 1197-1204 |
| | | semialdehyde dehydrogenase | (1991); Kalinowski, J. et al. “Aspartokinase |
| | | | genes lysC alpha and lysC beta overlap and are |
| | | | adjacent to the aspertate beta-semialdehyde |
| | | | dehydrogenase gene asd in Corynebacterium |
| | | | glutamicum,” Mol. Gen. Genet., |
| | | | 224(3): 317-324 (1990) |
| X59403 | gap; pgk; tpi | Glyceraldehyde-3-phosphate; | Eikmanns, B. J. “Identification, sequence |
| | | phosphoglycerate kinase; | analysis, and expression of a Corynebacterium |
| | | triosephosphate isomerase | glutamicum gene cluster encoding the three |
| | | | glycolytic enzymes glyceraldehyde-3-phosphate |
| | | | dehydrogenase, 3-phosphoglycerate |
| | | | kinase, and triosephosphate isomeras,” |
| | | | J. Bacteriol., 174(19): 6076-6086 (1992) |
| X59404 | gdh | Glutamate dehydrogenase | Bormann, E. R. et al. “Molecular analysis of |
| | | | the Corynebacterium glutamicum gdh gene encoding |
| | | | glutamate dehydrogenase,” Mol. Microbiol., |
| | | | 6(3): 317-326 (1992) |
| X60312 | lysl | L-lysine permease | Seep-Feldhaus, A. H. et al. “Molecular |
| | | | analysis of the Corynebacterium glutamicum lysl |
| | | | gene involved in lysine uptake,” Mol. |
| | | | Microbiol., 5(12): 2995-3005 (1991) |
| X66078 | cop1 | Ps1 protein | Joliff, G. et al. “Cloning and nucleotide |
| | | | sequence of the csp 1 gene encoding PS1, one of |
| | | | the two major secreted proteins of |
| | | | Corynebacterium glutamicum: The deduced N-terminal |
| | | | region of PS1 is similar to the Mycobacterium |
| | | | antigen 85 complex,” Mol. Microbiol., |
| | | | 6(16): 2349-2362 (1992) |
| X66112 | glt | Citrate synthase | Eikmanns, B. J. et al. “Cloning sequence, |
| | | | expression and transcriptional analysis of the |
| | | | Corynebacterium glutamicum gltA gene encoding |
| | | | citrate synthase,” Microbiol., |
| | | | 140: 1817-1828 (1994) |
| X67737 | dapB | Dihydrodipicolinate reductase | |
| X69103 | csp2 | Surface layer protein PS2 | Peyret, J. L. et al. “Characterization of the |
| | | | cspB gene encoding PS2, an ordered surface-layer |
| | | | protein in Corynebacterium glutamicum,” Mol. |
| | | | Microbiol., 9(1): 97-109 (1993) |
| X69104 | | IS3 related insertion element | Bonamy, C. et al. “Identification of IS1206, |
| | | | a Corynebacterium glutamicum IS3-related insertion |
| | | | sequence and phylogenetic analysis,” Mol. |
| | | | Microbiol., 14(3): 571-581 (1994) |
| X70959 | leuA | Isopropylmalate synthase | Patek, M. et al. “Leucine synthesis in |
| | | | Corynebacterium glutamicum: enzyme activities, |
| | | | structure of leuA, and effect of leuA inactivation |
| | | | on lysine synthesis,” Appl. Environ. |
| | | | Microbiol., 60(1): 133-140 (1994) |
| X71489 | icd | Isocitrate dehydrogenase | Eikmanns, B. J. et al. “Cloning sequence |
| | | (NADP+) | analysis, expression, and inactivation of the |
| | | | Corynebacterium glutamicum icd gene encoding |
| | | | isocitrate dehydrogenase and biochemical |
| | | | characterization of the enzyme,” J. Bacteriol., |
| | | | 177(3): 774-782 (1995) |
| X72855 | GDHA | Glutamate dehydrogenase (NADP+) | |
| X75083, | mtrA | 5-methyltryptophan resistance | Heery, D. M. et al. “A sequence from a |
| X70584 | | | tryptophan-hyperproducing strain of |
| | | | Corynebacterium glutamicum encoding resistance |
| | | | to 5-methyltryptophan,” Biochem. Biophys. |
| | | | Res. Commun., 201(3): 1255-1262 (1994) |
| X75085 | recA | | Fitzpatrick, R. et al. “Construction and |
| | | | characterization of recA mutant strains |
| | | | of Corynebacterium glutamicum and |
| | | | Brevibacterium lactofermentum,” Appl. |
| | | | Microbioil. Biotechnol., 42(4): 575-580 (1994) |
| X75504 | aceA; thiX | Partial Isocitrate lyase; ? | Reinscheid, D. J. et al. “Characterization of |
| | | | the isocitrate lyase gene from |
| | | | Corynebacterium glutamicum and biochemical |
| | | | analysis of the enzyme,” J. |
| | | | Bacterial., 176(12): 3474-3483 (1994) |
| X76875 | | ATPase beta-subunit | Ludwig, W. et al. “Phylogenetic relationships |
| | | | of bacteria based on comparative sequence analysis |
| | | | of elongation factor Tu and ATP-synthase beta- |
| | | | subunit genes,” Antonie Van Leeuwenhoek, |
| | | | 64: 285-305 (1993) |
| X77034 | tuf | Elongation factor Tu | Ludwig, W. et al. “Phylogenetic relationships |
| | | | of bacteria based on comparative sequence analysis |
| | | | of elongation factor Tu and ATP-synthase beta- |
| | | | subunit genes,” Antonie Van Leeuwenhoek, |
| | | | 64: 285-305 (1993) |
| X77384 | recA | | Billman-Jacobe, H. “Nucleotide sequence of a |
| | | | recA gene from Corynebacterium glutamicum,” |
| | | | DNA Seq., 4(6): 403-404 (1994) |
| X78491 | aceB | Malate synthase | Reinscheid, D. J. et al. “Malate synthase from |
| | | | Corynebacterium glutamicum pta-ack operon |
| | | | encoding phosphotransacetylase: sequence |
| | | | analysis,” Microbiology, 140: 3099-3108 (1994) |
| X80629 | 16S rDNA | 16S ribosomal RNA | Rainey, F. A. et al. “Phylogenetic analysis of |
| | | | the genera Rhodococcus and Norcardia and evidence |
| | | | for the evolutionary origin of the genus |
| | | | Norcardia from within the radiation of |
| | | | Rhodococcus species,” Microbiol, 141: 523-528 |
| | | | (1995) |
| X81191 | gluA; gluB; | Glutamate uptake system | Kronemeyer, W. et al. “Structure of the |
| | gluC; gluD | | gluABCD cluster encoding the glutamate uptake |
| | | | system of Corynebacterium glutamicum,” J. |
| | | | Bacteriol., 177(5): 1152-1158 (1995) |
| X81379 | dapE | Succinyldiaminopimelate | Wehrmann, A. et al. “Analysis of different |
| | | desuccinylase | DNA fragments of Corynebacterium glutamicum |
| | | | complementing dapE of Escherichia coli,” |
| | | | Microbiology, 40: 3349-56 (1994) |
| X82061 | 16S rDNA | 16S ribosomal RNA | Ruimy, R. et al. “Phylogeny of the genus |
| | | | Corynebacterium deduced from analyses of small- |
| | | | subunit ribosomal DNA sequences,” Int. J. |
| | | | Syst. Bacteriol., 45(4): 740-746 (1995) |
| X82928 | asd; lysC | Aspartate-semialdehyde | Serebrijski, I. et al. “Multicopy suppression |
| | | dehydrogenase; ? | by asd gene and osmotic stress-dependent |
| | | | complementation by heterologous proA in proA |
| | | | mutants,” J. Bacteriol., 177(24): 7255-7260 (1995) |
| X82929 | proA | Gamma-glutamyl phosphate reductase | Serebrijski, I. et al. “Multicopy suppression |
| | | | by asd gene and osmotic stress-dependent |
| | | | complementation by heterologous proA in proA |
| | | | mutants,” J. Bacteriol., 177(24): 7255-7260 (1995) |
| X84257 | 16S rDNA | 16S ribosomal RNA | Pascual, C. et al. “Phylogenetic analysis of |
| | | | the genus Corynebacterium based on 16S rRNA gene |
| | | | sequences,” Int. J. Syst. Bacteriol., |
| | | | 45(4): 724-728 (1995) |
| X85965 | aroP; dapE | Aromatic amino acid permease; ? | Wehrmann, A. et al. “Functional analysis of |
| | | | sequences adjacent to dapE of |
| | | | Corynebacterium glutamicum proline reveals the |
| | | | presence of aroP, which encodes the aromatic |
| | | | amino acid transporter,” J. Bacteriol., |
| | | | 177(20): 5991-5993 (1995) |
| X86157 | argB; argC; | Acetylglutamate kinase; | Sakanyan, V. et al. “Genes and enzymes of |
| | argD; argF; | N-acetyl-gamma-glutamyl-phosphate | the acetyl cycle of arginine biosynthesis in |
| | argJ | reductase; acetylornithine | Corynebacterium glutamicum: enzyme evolution in |
| | | aminotransferase; ornithine | the early steps of the arginine pathway,” |
| | | carbamoyltransferase; glutamate N- | Microbiology, 142: 99-108 (1996) |
| | | acetyltransferase | |
| X89084 | pta; ackA | Phosphate acetyltransferase; | Reinscheid, D. J. et al. “Cloning, sequence |
| | | acetate kinase | analysis, expression and inactivation of the |
| | | | Corynebacterium glutamicum pta-ack operon encoding |
| | | | phosphotransacetylase and acetate kinase,” |
| | | | Microbiology, 145: 503-513 (1999) |
| X89850 | attB | Attachment site | Le Marrec, C. et al. “Genetic characterization of site-specific integration |
| | | | functions of phi AAU2 infecting ”Arthrobacter |
| | | | aureus C70,” J. Bacteriol., |
| | | | 178(7): 1996-2004 (1996) |
| X90356 | | Promoter fragment F1 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90357 | | Promoter fragment F2 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90358 | | Promoter fragment F10 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90359 | | Promoter fragment F13 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90360 | | Promoter fragment F22 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90361 | | Promoter fragment F34 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90362 | | Promoter fragment F37 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90363 | | Promoter fragment F45 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90364 | | Promoter fragment F64 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90365 | | Promoter fragment F75 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90366 | | Promoter fragment PF101 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90367 | | Promoter fragment PF104 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X90368 | | Promoter fragment PF109 | Patek, M. et al. “Promoters from |
| | | | Corynebacterium glutamicum: cloning, molecular |
| | | | analysis and search for a consensus motif,” |
| | | | Microbiology, 142: 1297-1309 (1996) |
| X93513 | amt | Ammonium transport system | Siewe, R. M. et al. “Functional and genetic |
| | | | characterization of the (methyl) ammonium |
| | | | uptake carrier of Corynebacterium glutamicum,” |
| | | | J. Biol. Chem., 271(10): 5398-5403 (1996) |
| X93514 | betP | Glycine betaine transport system | Peter, H. et al. “Isolation, characterization, |
| | | | and expression of the Corynebacterium glutamicum |
| | | | betP gene, encoding the transport system for the |
| | | | compatible solute glycine betaine,” |
| | | | J. Bacteriol., 178(17): 5229-5234 (1996) |
| X95649 | orf4 | | Patek, M. et al. “Identification and |
| | | | transcriptional analysis of the dapB-ORF2- |
| | | | dapA-ORF4 operon of Corynebacterium glutamicum, |
| | | | encoding two enzymes involved in L-lysine |
| | | | synthesis,” Biotechnol. Lett., |
| | | | 19: 1113-1117 (1997) |
| X96471 | lysE; lysG | Lysine exporter protein; | Vrljic, M. et al. “A new type of transporter |
| | | Lysine export regulator protein | with a new type of cellular function: L-lysine |
| | | | export from Corynebacterium glutamicum,” Mol. |
| | | | Microbiol., 22(5): 815-826 (1996) |
| X96580 | panB; panC; | 3-methyl-2-oxobutanoate | Sahm, H. et al. “D-pantothenate synthesis in |
| | xylB | hydroxymethyltransferase; | Corynebacterium glutamicum and use of panBC and |
| | | pantoate-beta-alanine ligase; | genes encoding L-valine synthesis for |
| | | xylulokinase | D-pantothenate overproduction,” Appl. Environ. |
| | | | Microbiol., 65(5): 1973-1979 (1999) |
| X96962 | | Insertion sequence IS 1207 | |
| | | and transposase | |
| X99289 | | Elongation factor P | Ramos, A. et al. “Cloning, sequencing and |
| | | | expression of the gene encoding elongation factor |
| | | | P in the amino-acid producer Brevibacterium |
| | | | lactofermentum (Corynebacterium glutamicum ATCC |
| | | | 13869),” Gene, 198: 217-222 (1997) |
| Y00140 | thrB | Homoserine kinase | Mateos, L. M. et al. “Nucleotide sequence of |
| | | | the homoserine kinase (thrB) gene of the |
| | | | Brevibacterium lactofermentum,” Nucleic Acids |
| | | | Res., 15(9): 3922 (1987) |
| Y00151 | ddh | Meso-diaminopimelate D- | Ishino, S. et al. “Nucleotide sequence of the |
| | | dehydrogenase (EC 1.4.1.16) | meso-diaminopimelate D-dehydrogenase gene from |
| | | | Corynebacterium glutamicum,” Nucleic Acids |
| | | | Res., 15(9): 3917(1987) |
| Y00476 | thrA | Homoserine dehydrogenase | Mateos, L. M. et al. “Nucleotide sequence of |
| | | | the homoserine dehydrogenase (thrA) gene of the |
| | | | Brevibacterium lactofermentum,” Nucleic Acids |
| | | | Res., 15(24): 10598 (1987) |
| Y00546 | hom; thrB | Homoserine dehydrogenase; | Peoples, O.P. et al. “Nucleotide sequence and |
| | | homoserine kinase | fine structural analysis of the |
| | | | Corynebacterium glutamicum hom-thrB operon,” |
| | | | Mol. Microbiol., 2(l): 63-72 (1988) |
| Y08964 | murC; ftsQ/ | UPD-N-acetylmuramate-alanine | Honrubia, M. P. et al. “Identification, |
| | divD; ftsZ | ligase; division initiation | characterization, and chromosomal organization of |
| | | protein or cell division | the ftsZ gene from Brevibacterium lactofermentum,” |
| | | protein; cell division protein | Mol. Gen. Genet., 259(1): 97-104 (1998) |
| Y09163 | putP | High affinity proline transport | Peter, H. et al. “Isolation of the putP gene |
| | | system | of Corynebacterium glutamicum proline and |
| | | | characterization of a low-affinity uptake system |
| | | | for compatible solutes” Arch. Microbiol., |
| | | | 168(2): 143-151 (1997) |
| Y09548 | pyc | Pyruvate carboxylase | Peters-Wendisch, P. G. et al. “Pyruvate |
| | | | carboxylase from Corynebacterium glutamicum: |
| | | | characterization, expression and inactivation of |
| | | | the pyc gene,” Microbiology, 144: 915-927 (1998) |
| Y09578 | ieuB | 3-isopropylmalate dehydrogenase | Patek, M. et al. “Analysis of the leuB gene |
| | | | from Corynebacterium glutamicum,” Appl. |
| | | | Microbiol. Biotechnol, 50(1): 42-47 (1998) |
| Y12472 | | Attachment site bacteriophage | Moreau, S. et al. “Site-specific integration |
| | | Phi-16 | of corynephage Phi-16: The construction of an |
| | | | integration vector” Microbiol., |
| | | | 145: 539-548 (1999) |
| Y12537 | proP | Proline/ectoine uptake system | Peter, H. et al. “Corynebacterium glutamicum |
| | | protein | is equipped with four secondary carriers for |
| | | | compatible solutes: Identification, sequencing, |
| | | | and characterization of the proline/ectoine |
| | | | uptake system, ProP, and the ectoine/proline/ |
| | | | glycine betaine carrier, EctP,” J. Bacteriol., |
| | | | 180(22): 6005-6012 (1998) |
| Y13221 | glnA | Glutamine synthetase I | Jakoby, M. et al. “Isolation of |
| | | | Corynebacterium glutamicum glnA gene |
| | | | encoding glutamine synthetase I,” FEMS |
| | | | Microbiol. Lett., 154(1): 81-88 (1997) |
| Y16642 | lpd | Dihydrolipoamide dehydrogenase | |
| Y18059 | | Attachment site Corynephage 304L | Moreau, S. et al. “Analysis of the integration |
| | | | functions of &phi; 304L: An integrase module |
| | | | among corynephages,” Virology, 255(1): 150-159 |
| | | | (1999) |
| Z21501 | argS; lysA | Arginyl-tRNA synthetase; | Oguiza, J. A. et al. “A gene encoding |
| | | diaminopimelate | arginyl-tRNA synthetase is located in the |
| | | decarboxylase (partial) | upstream region of the lysA gene in |
| | | | Brevibacterium lactofermentum: Regulation of |
| | | | argS-lysA cluster expression by arginine,” J. |
| | | | Bacteriol., 175(22): 7356-7362 (1993) |
| Z21502 | dapA; dapB | Dihydrodipicolinate synthase; | Pisabarro, A. et al. “A cluster of three genes |
| | | dihydrodipicolinate reductase | (dapA, orf2, and dapB) of |
| | | | Brevibacterium lactofermentum encodes |
| | | | dihydrodipicolinate reductase, and a third |
| | | | polypeptide of unknown function,” J. |
| | | | Bacterial., 175(9): 2743-2749 (1993) |
| Z29563 | thrC | Threonine synthase | Malumbres, M. et al. “Analysis and expression |
| | | | of the thrC gene of the encoded threonine |
| | | | synthase,” Appl. Environ. Microbiol., |
| | | | 60(7)2209-2219 (1994) |
| Z46753 | 16S rDNA | Gene for 16S ribosomal RNA | |
| Z49822 | sigA | SigA sigma factor | Oguiza, J. A. et al “Multiple sigma factor |
| | | | genes in Brevibacterium lactofermentum: |
| | | | Characterization of sigA and sigB,” J. |
| | | | Bacteriol., 178(2): 550-553 (1996) |
| Z49823 | galE; dtxR | Catalytic activity UDP-galactose | Oguiza, J. A. et al “The galE gene encoding the |
| | | 4-epimerase; diphtheria toxin | UDP-galactose 4-epimerase of Brevibacterium |
| | | regulatory protein | lactofermentumis coupled transcriptionally to |
| | | | the dmdR gene,” Gene, 177: 103-107 (1996) |
| Z49824 | orf1; sigB | ?; SigB sigma factor | Oguiza, J. A. et al “Multiple sigma factor |
| | | | genes in Brevibacterium lactofermentum: |
| | | | Characterization of sigA and sigB,” J. |
| | | | Bacterial., 178(2): 550-553(1996) |
| Z66534 | | Transposase | Correia, A. et al. “Cloning and |
| | | | characterization of an IS-like element present |
| | | | in the genome of Brevibacterium lactofermentum |
| | | | ATCC 13869,” Gene, 170(1): 91-94 (1996) |

¹A sequence for this gene was published in the indicated reference. However, the sequence obtained by the inventors of the present application is significantly longer than the published version. It is believed that the published version relied on an incorrect start codon, and thus represents only a fragment of the actual coding region.

TABLE 3
Corynebacterium and Brevibacterium Strains Which May be Used in the Practice of the Invention

| Genus | species | ATCC | FERM | NRRL | CECT | NCIMB | CBS | NCTC | DSMZ |
|---|---|---|---|---|---|---|---|---|---|
| Brevibacterium | ammoniagenes | 21054 | | | | | | | |
| Brevibacterium | ammoniagenes | 19350 | | | | | | | |
| Brevibacterium | ammoniagenes | 19351 | | | | | | | |
| Brevibacterium | ammoniagenes | 19352 | | | | | | | |
| Brevibacterium | ammoniagenes | 19353 | | | | | | | |
| Brevibacterium | ammoniagenes | 19354 | | | | | | | |
| Brevibacterium | ammoniagenes | 19355 | | | | | | | |
| Brevibacterium | ammoniagenes | 19356 | | | | | | | |
| Brevibacterium | ammoniagenes | 21055 | | | | | | | |
| Brevibacterium | ammoniagenes | 21077 | | | | | | | |
| Brevibacterium | ammoniagenes | 21553 | | | | | | | |
| Brevibacterium | ammoniagenes | 21580 | | | | | | | |
| Brevibacterium | ammoniagenes | 39101 | | | | | | | |
| Brevibacterium | butanicum | 21196 | | | | | | | |
| Brevibacterium | divaricatum | 21792 | P928 | | | | | | |
| Brevibacterium | flavum | 21474 | | | | | | | |
| Brevibacterium | flavum | 21129 | | | | | | | |
| Brevibacterium | flavum | 21518 | | | | | | | |
| Brevibacterium | flavum | | | B11474 | | | | | |
| Brevibacterium | flavum | | | B11472 | | | | | |
| Brevibacterium | flavum | 21127 | | | | | | | |
| Brevibacterium | flavum | 21128 | | | | | | | |
| Brevibacterium | flavum | 21427 | | | | | | | |
| Brevibacterium | flavum | 21475 | | | | | | | |
| Brevibacterium | flavum | 21517 | | | | | | | |
| Brevibacterium | flavum | 21528 | | | | | | | |
| Brevibacterium | flavum | 21529 | | | | | | | |
| Brevibacterium | flavum | | | B11477 | | | | | |
| Brevibacterium | flavum | | | B11478 | | | | | |
| Brevibacterium | flavum | 21127 | | | | | | | |
| Brevibacterium | flavum | | | B11474 | | | | | |
| Brevibacterium | healii | 15527 | | | | | | | |
| Brevibacterium | ketoglutamicum | 21004 | | | | | | | |
| Brevibacterium | ketoglutamicum | 21089 | | | | | | | |
| Brevibacterium | ketosoreductum | 21914 | | | | | | | |
| Brevibacterium | lactofermentum | | | | 70 | | | | |
| Brevibacterium | lactofermentum | | | | 74 | | | | |
| Brevibacterium | lactofermentum | | | | 77 | | | | |
| Brevibacterium | lactofermentum | 21798 | | | | | | | |
| Brevibacterium | lactofermentum | 21799 | | | | | | | |
| Brevibacterium | lactofermentum | 21800 | | | | | | | |
| Brevibacterium | lactofermentum | 21801 | | | | | | | |
| Brevibacterium | lactofermentum | | | B11470 | | | | | |
| Brevibacterium | lactofermentum | | | B11471 | | | | | |
| Brevibacterium | lactofermentum | 21086 | | | | | | | |
| Brevibacterium | lactofermentum | 21420 | | | | | | | |
| Brevibacterium | lactofermentum | 21086 | | | | | | | |
| Brevibacterium | lactofermentum | 31269 | | | | | | | |
| Brevibacterium | linens | 9174 | | | | | | | |
| Brevibacterium | linens | 19391 | | | | | | | |
| Brevibacterium | linens | 8377 | | | | | | | |
| Brevibacterium | paraffinolyticum | | | | | 11160 | | | |
| Brevibacterium | spec. | | | | | | 717.73 | | |
| Brevibacterium | spec. | | | | | | 717.73 | | |
| Brevibacterium | spec. | 14604 | | | | | | | |
| Brevibacterium | spec. | 21860 | | | | | | | |
| Brevibacterium | spec. | 21864 | | | | | | | |
| Brevibacterium | spec. | 21865 | | | | | | | |
| Brevibacterium | spec. | 21866 | | | | | | | |
| Brevibacterium | spec. | 19240 | | | | | | | |
| Corynebacterium | acetoacidophilum | 21476 | | | | | | | |
| Corynebacterium | acetoacidophilum | 13870 | | | | | | | |
| Corynebacterium | ammoniagenes | | | B11473 | | | | | |
| Corynebacterium | ammoniagenes | | | B11475 | | | | | |
| Corynebacterium | ammoniagenes | 15806 | | | | | | | |
| Corynebacterium | ammoniagenes | 21491 | | | | | | | |
| Corynebacterium | ammoniagenes | 31270 | | | | | | | |
| Corynebacterium | acetophilum | | | B3671 | | | | | |
| Corynebacterium | ammoniagenes | 6872 | | | | | | 2399 | |
| Corynebacterium | ammoniagenes | 15511 | | | | | | | |
| Corynebacterium | fujiokense | 21496 | | | | | | | |
| Corynebacterium | glutamicum | 14067 | | | | | | | |
| Corynebacterium | glutamicum | 39137 | | | | | | | |
| Corynebacterium | glutamicum | 21254 | | | | | | | |
| Corynebacterium | glutamicum | 21255 | | | | | | | |
| Corynebacterium | glutamicum | 31830 | | | | | | | |
| Corynebacterium | glutamicum | 13032 | | | | | | | |
| Corynebacterium | glutamicum | 14305 | | | | | | | |
| Corynebacterium | glutamicum | 15455 | | | | | | | |
| Corynebacterium | glutamicum | 13058 | | | | | | | |
| Corynebacterium | glutamicum | 13059 | | | | | | | |
| Corynebacterium | glutamicum | 13060 | | | | | | | |
| Corynebacterium | glutamicum | 21492 | | | | | | | |
| Corynebacterium | glutamicum | 21513 | | | | | | | |
| Corynebacterium | glutamicum | 21526 | | | | | | | |
| Corynebacterium | glutamicum | 21543 | | | | | | | |
| Corynebacterium | glutamicum | 13287 | | | | | | | |
| Corynebacterium | glutamicum | 21851 | | | | | | | |
| Corynebacterium | glutamicum | 21253 | | | | | | | |
| Corynebacterium | glutamicum | 21514 | | | | | | | |
| Corynebacterium | glutamicum | 21516 | | | | | | | |
| Corynebacterium | glutamicum | 21299 | | | | | | | |
| Corynebacterium | glutamicum | 21300 | | | | | | | |
| Corynebacterium | glutamicum | 39684 | | | | | | | |
| Corynebacterium | glutamicum | 21488 | | | | | | | |
| Corynebacterium | glutamicum | 21649 | | | | | | | |
| Corynebacterium | glutamicum | 21650 | | | | | | | |
| Corynebacterium | glutamicum | 19223 | | | | | | | |
| Corynebacterium | glutamicum | 13869 | | | | | | | |
| Corynebacterium | glutamicum | 21157 | | | | | | | |
| Corynebacterium | glutamicum | 21158 | | | | | | | |
| Corynebacterium | glutamicum | 21159 | | | | | | | |
| Corynebacterium | glutamicum | 21355 | | | | | | | |
| Corynebacterium | glutamicum | 31808 | | | | | | | |
| Corynebacterium | glutamicum | 21674 | | | | | | | |
| Corynebacterium | glutamicum | 21562 | | | | | | | |
| Corynebacterium | glutamicum | 21563 | | | | | | | |
| Corynebacterium | glutamicum | 21564 | | | | | | | |
| Corynebacterium | glutamicum | 21565 | | | | | | | |
| Corynebacterium | glutamicum | 21566 | | | | | | | |
| Corynebacterium | glutamicum | 21567 | | | | | | | |
| Corynebacterium | glutamicum | 21568 | | | | | | | |
| Corynebacterium | glutamicum | 21569 | | | | | | | |
| Corynebacterium | glutamicum | 21570 | | | | | | | |
| Corynebacterium | glutamicum | 21571 | | | | | | | |
| Corynebacterium | glutamicum | 21572 | | | | | | | |
| Corynebacterium | glutamicum | 21573 | | | | | | | |
| Corynebacterium | glutamicum | 21579 | | | | | | | |
| Corynebacterium | glutamicum | 19049 | | | | | | | |
| Corynebacterium | glutamicum | 19050 | | | | | | | |
| Corynebacterium | glutamicum | 19051 | | | | | | | |
| Corynebacterium | glutamicum | 19052 | | | | | | | |
| Corynebacterium | glutamicum | 19053 | | | | | | | |
| Corynebacterium | glutamicum | 19054 | | | | | | | |
| Corynebacterium | glutamicum | 19055 | | | | | | | |
| Corynebacterium | glutamicum | 19056 | | | | | | | |
| Corynebacterium | glutamicum | 19057 | | | | | | | |
| Corynebacterium | glutamicum | 19058 | | | | | | | |
| Corynebacterium | glutamicum | 19059 | | | | | | | |
| Corynebacterium | glutamicum | 19060 | | | | | | | |
| Corynebacterium | glutamicum | 19185 | | | | | | | |
| Corynebacterium | glutamicum | 13286 | | | | | | | |
| Corynebacterium | glutamicum | 21515 | | | | | | | |
| Corynebacterium | glutamicum | 21527 | | | | | | | |
| Corynebacterium | glutamicum | 21544 | | | | | | | |
| Corynebacterium | glutamicum | 21492 | | | | | | | |
| Corynebacterium | glutamicum | | | B8183 | | | | | |
| Corynebacterium | glutamicum | | | B8182 | | | | | |
| Corynebacterium | glutamicum | | | B12416 | | | | | |
| Corynebacterium | glutamicum | | | B12417 | | | | | |
| Corynebacterium | glutamicum | | | B12418 | | | | | |
| Corynebacterium | glutamicum | | | B11476 | | | | | |
| Corynebacterium | glutamicum | 21608 | | | | | | | |
| Corynebacterium | lilium | | P973 | | | | | | |
| Corynebacterium | nitrilophilus | 21419 | | | | 11594 | | | |
| Corynebacterium | spec. | | P4445 | | | | | | |
| Corynebacterium | spec. | | P4446 | | | | | | |
| Corynebacterium | spec. | 31088 | | | | | | | |
| Corynebacterium | spec. | 31089 | | | | | | | |
| Corynebacterium | spec. | 31090 | | | | | | | |
| Corynebacterium | spec. | 31090 | | | | | | | |
| Corynebacterium | spec. | 31090 | | | | | | | |
| Corynebacterium | spec. | 15954 | | | | | | | 20145 |
| Corynebacterium | spec. | 21857 | | | | | | | |
| Corynebacterium | spec. | 21862 | | | | | | | |
| Corynebacterium | spec. | 21863 | | | | | | | |

ATCC: American Type Culture Collection, Rockville, MD, USA
FERM: Fermentation Research Institute, Chiba, Japan
NRRL: ARS Culture Collection, Northern Regional Research Laboratory, Peoria, IL, USA
CECT: Coleccion Espanola de Cultivos Tipo, Valencia, Spain
NCIMB: National Collection of Industrial and Marine Bacteria Ltd., Aberdeen, UK
CBS: Centraalbureau voor Schimmelcultures, Baarn, NL
NCTC: National Collection of Type Cultures, London, UK
DSMZ: Deutsche Sammlung von Mikroorganismen und Zellkulturen, Braunschweig, Germany
For reference see Sugawara, H. et al. (1993) World directory of collections of cultures of microorganisms: Bacteria, fungi and yeasts (4^{th} edn), World federation for culture collections world data center on microorganisms, Saimata, Japen.

TABLE 4
ALIGNMENT RESULTS

| | | | | | | | % | |
|---|---|---|---|---|---|---|---|---|
| | | | | | | | homol- | |
| | length | Genbank | | Acces- | | Source of | ogy | Date of |
| ID # | (NT) | Hit | Length | sion | Name of Genbank Hit | Genbank Hit | (GAP) | Deposit |
| rxa00004 | 594 | GB_IN1: | 34660 | U58762 | Caenorhabditis elegans | Caenorhabditis | 36,442 | 24-MAY-1996 |
| | | CELT27F7 | | | cosmid T27F7. | elegans | | |
| | | GB_PR4: | 161910 | AC005531 | Homo sapiens PAC | Homo sapiens | 36,672 | 13-Jan-99 |
| | | AC005531 | | | clone DJ0701O16 | | | |
| | | | | | from 7q33-q36, | | | |
| | | | | | complete sequence. | | | |
| | | GB_EST36: | 360 | AV186136 | AV186136 Yuji Kohara | Caenorhabditis | 44,380 | 22-Jul-99 |
| | | AV186136 | | | unpublished cDNA: | elegans | | |
| | | | | | Strain N2 hermaphrodite | | | |
| | | | | | embryo | | | |
| | | | | | Caenorhabditis elegans | | | |
| | | | | | cDNA clone yk495f12 | | | |
| | | | | | 5′, mRNA sequence. | | | |
| rxa00006 | 558 | GB_BA1: | 8734 | AB024708 | Corynebacterium | Corynebacterium | 39,525 | 13-MAR-1999 |
| | | AB024708 | | | glutamicum gltB and | glutamicum | | |
| | | | | | gltD genes for | | | |
| | | | | | glutamine | | | |
| | | | | | 2-oxoglutarate | | | |
| | | | | | aminotransferase | | | |
| | | | | | large and small | | | |
| | | | | | subunits, complete cds. | | | |
| | | GB_EST5: | 434 | N23892 | yw46f12.s1 Weizmann | Homo sapiens | 38,462 | 28-DEC-1995 |
| | | N23892 | | | Olfactory Epithelium | | | |
| | | | | | Homo sapiens cDNA clone | | | |
| | | | | | IMAGE: 255311 3′, | | | |
| | | | | | mRNA sequence. | | | |
| | | GB_BA1: | 8734 | AB024708 | Corynebacterium | Corynebacterium | 38,961 | 13-MAR-1999 |
| | | AB024708 | | | glutamicum gltB and | glutamicum | | |
| | | | | | gltD genes for glutamine | | | |
| | | | | | 2-oxoglutarate | | | |
| | | | | | aminotransferase | | | |
| | | | | | large and small | | | |
| | | | | | subunits, complete cds. | | | |
| rxa00029 | | | | | | | | |
| rxa00126 | | | | | | | | |
| rxa00129 | 1620 | GB_BA1: | 36330 | Z95121 | Mycobacterium | Mycobacterium | 40,788 | 17-Jun-98 |
| | | MTY20B11 | | | tuberculosis H37Rv | tuberculosis | | |
| | | | | | complete genome; | | | |
| | | | | | segment 139/162. | | | |
| | | GB_BA1: | 1799 | U14909 | Mycobacterium | Mycobacterium | 54,422 | 11-Sep-96 |
| | | MTU14909 | | | tuberculosis MtrB | tuberculosis | | |
| | | | | | (mtrB) gene, | | | |
| | | | | | complete cds. | | | |
| | | GB_HTG2: | 140702 | AC006888 | Caenorhabditis elegans | Caenorhabditis | 35,883 | 26-Feb-99 |
| | | AC006888 | | | clone | elegans | | |
| | | | | | Y61A9L, | | | |
| | | | | | *** SEQUENCING | | | |
| | | | | | IN PROGRESS ***, | | | |
| | | | | | 2 unordered pieces. | | | |
| rxa00130 | 801 | GB_BA1: | 36330 | Z95121 | Mycobacterium | Mycobacterium | 41,069 | 17-Jun-98 |
| | | MTY20B11 | | | tuberculosis | tuberculosis | | |
| | | | | | H37Rv complete | | | |
| | | | | | genome; segment 139/162. | | | |
| | | GB_BA1: | 689 | U01971 | Mycobacterium | Mycobacterium | 66,183 | 11-Sep-96 |
| | | MTU01971 | | | tuberculosis | tuberculosis | | |
| | | | | | H37Rv MtrA | | | |
| | | | | | (mtrA) gene, | | | |
| | | | | | complete cds. | | | |
| | | GB_BA1: | 618 | X92405 | N. meningitidis | Neisseria | 50,249 | 31-OCT-1995 |
| | | NMOMPR | | | ompR gene. | meningitidis | | |
| rxa00182 | 3225 | GB_BA1: | 3791 | Y09163 | C. glutamicum | Corynebacterium | 41,126 | 8-Sep-97 |
| | | CGPUTP | | | putP gene. | glutamicum | | |
| | | GB_BA1: | 5143 | AL021924 | Mycobacterium | Mycobacterium | 48,140 | 17-Jun-98 |
| | | MTV020 | | | tuberculosis | tuberculosis | | |
| | | | | | H37Rv complete | | | |
| | | | | | genome; segment | | | |
| | | | | | 94/162. | | | |
| | | GB_BA1: | 212610 | Z99122 | Bacillus subtilis | Bacillus | 44,221 | 24-Jun-99 |
| | | BSUB0019 | | | complete genome | subtilis | | |
| | | | | | (section 19 of 21): | | | |
| | | | | | from 3597091 to | | | |
| | | | | | 3809700. | | | |
| rxa00221 | 342 | GB_PL2: | 1415 | AF020584 | Welwitschia mirabilis | Mitochondrion | 36,656 | 5-Jan-99 |
| | | AF020584 | | | cytochrome c oxidase | Welwitschia | | |
| | | | | | (coxl) gene, | mirabilis | | |
| | | | | | mitochondrial gene | | | |
| | | | | | encoding mitochondrial | | | |
| | | | | | protein, partial cds. | | | |
| | | GB_PR4: | 95240 | AC007421 | Homo sapiens chromosome | Homo sapiens | 35,061 | 27-Aug-99 |
| | | AC007421 | | | 17, clone hRPC.1030_O_14, | | | |
| | | | | | complete sequence. | | | |
| | | GB_BA2: | 60232 | AE001272 | Lactococcus lactis | Lactococcus | 37,764 | 11-Sep-98 |
| | | AE001272 | | | DPC3147 plasmid | lactis | | |
| | | | | | pMRC01, complete | | | |
| | | | | | plasmid sequence. | | | |
| rxa00253 | 861 | GB_BA2: | 1638 | AF126953 | Corynebacterium | Corynebacterium | 41,107 | 10-Sep-99 |
| | | AF126953 | | | glutamicum | glutamicum | | |
| | | | | | cystathionine | | | |
| | | | | | gamma-synthase | | | |
| | | | | | (metB) gene, | | | |
| | | | | | complete cds. | | | |
| | | GB_PR3: | 148440 | AL096791 | Human DNA sequence | Homo sapiens | 36,190 | 23-Nov-99 |
| | | HSJ659F15 | | | from clone 659F15 | | | |
| | | | | | on chromosome | | | |
| | | | | | Xp11.21-11.4, | | | |
| | | | | | complete sequence. | | | |
| | | GB_HTG1: | 129149 | Z98044 | Homo sapiens | Homo sapiens | 36,450 | 23-Nov-99 |
| | | HS510D11 | | | chromosome 1 clone | | | |
| | | | | | RP3-510D11, | | | |
| | | | | | *** SEQUENCING | | | |
| | | | | | IN PROGRESS ***, in | | | |
| | | | | | unordered pieces. | | | |
| rxa00284 | 1188 | GB_PR2: | 108260 | Z98880 | Human DNA sequence | Homo sapiens | 38,370 | 23-Nov-99 |
| | | HS179P9 | | | from PAC 179P9 on | | | |
| | | | | | chromosome 6q22. | | | |
| | | | | | Contains transmembrane | | | |
| | | | | | tyrosine-specific | | | |
| | | | | | protein kinase | | | |
| | | | | | (ROS1), ESTs and STS. | | | |
| | | GB_PR4: | 113345 | AF109076 | Homo sapiens | Homo sapiens | 35,340 | 13-DEC-1998 |
| | | AF109076 | | | chromosome 7 | | | |
| | | | | | map 7q36 BAC H6, | | | |
| | | | | | complete sequence. | | | |
| | | GB_PR2: | 108260 | Z98880 | Human DNA sequence | Homo sapiens | 35,344 | 23-Nov-99 |
| | | HS179P9 | | | from PAC 179P9 on | | | |
| | | | | | chromosome 6q22. | | | |
| | | | | | Contains transmembrane | | | |
| | | | | | tyrosine-specific | | | |
| | | | | | protein kinase | | | |
| | | | | | (ROS1), ESTs and STS. | | | |
| rxa00287 | 597 | GB_IN2: | 7887 | AF144549 | Aedes albopictus | Aedes | 39,828 | 3-Jun-99 |
| | | AF144549 | | | ribosomal protein | albopictus | | |
| | | | | | L34 (rpl34) gene, | | | |
| | | | | | complete cds. | | | |
| | | GB_EST15: | 503 | AA475366 | vh14e09.r1 Soares | Mus musculus | 37,063 | 18-Jun-97 |
| | | AA475366 | | | mouse mammary gland | | | |
| | | | | | NbMMG Mus musculus | | | |
| | | | | | cDNA clone | | | |
| | | | | | IMAGE: 875464 5′ | | | |
| | | | | | similar to gb: X87671 | | | |
| | | | | | M. musculus mRNA | | | |
| | | | | | for 3BP-1, an SH3 | | | |
| | | | | | domain binding | | | |
| | | | | | (MOUSE);, | | | |
| | | | | | mRNA sequence. | | | |
| | | GB_RO: | 2359 | X87671 | M. musculus mRNA for | Mus musculus | 34,635 | 20-OCT-1995 |
| | | MM3BP1 | | | 3BP-1, an SH3 domain | | | |
| | | | | | binding protein. | | | |
| rxa00291 | 1606 | GB_PR4: | 138107 | AC004967 | Homo sapiens clone | Homo sapiens | 36,785 | 5-Jun-99 |
| | | AC004967 | | | DJ1111F22, | | | |
| | | | | | complete sequence. | | | |
| | | GB_EST1: | 418 | M89319 | CEL21A4 Chris Martin | Caenorhabditis | 38,418 | 02-DEC-1992 |
| | | M89319 | | | sorted cDNA library | elegans | | |
| | | | | | Caenorhabditis elegans | | | |
| | | | | | cDNA clone | | | |
| | | | | | cm21a4 5′ similar to | | | |
| | | | | | pepsinogen A homologous | | | |
| | | | | | peptide, mRNA sequence. | | | |
| | | GB_GSS15: | 569 | AQ641399 | RPCI93-DpnII-28C1.TV | Trypanosoma | 39,106 | 8-Jul-99 |
| | | AQ641399 | | | RPCI93-DpnII | brucei | | |
| | | | | | Trypanosoma brucei | | | |
| | | | | | genomic clone | | | |
| | | | | | RPCI93-DpnII-28C1, | | | |
| | | | | | genomic survey | | | |
| | | | | | sequence. | | | |
| rxa00292 | 777 | GB_PL1: | 2112 | M34531 | S. cerevisiae | Saccharomyces | 37,330 | 27-Apr-93 |
| | | YSCKGD2 | | | dihydrolipoyl | cerevisiae | | |
| | | | | | transsuccinylase | | | |
| | | | | | (KGD2) gene, | | | |
| | | | | | complete cds. | | | |
| | | GB_PL1: | 9851 | X61236 | S. cerevisiae | Saccharomyces | 36,070 | 06-DEC-1991 |
| | | SCNUM1 | | | NUM1 gene, | cerevisiae | | |
| | | | | | involved in nuclear | | | |
| | | | | | migration control. | | | |
| | | GB_PL1: | 43468 | Z50046 | S. cerevisiae | Saccharomyces | 36,070 | 11-Aug-97 |
| | | SC8358 | | | chromosome IV | cerevisiae | | |
| | | | | | cosmid 8358. | | | |
| rxa00319 | 549 | GB_BA1: | 282700 | D84432 | Bacillus subtilis | Bacillus | 43,258 | 6-Feb-99 |
| | | BACJH642 | | | DNA, 283 Kb region | subtilis | | |
| | | | | | containing skin | | | |
| | | | | | element. | | | |
| | | GB_BA1: | 213420 | Z99117 | Bacillus subtilis | Bacillus | 34,264 | 26-Nov-97 |
| | | BSUB0014 | | | complete genome | subtilis | | |
| | | | | | (section 14 of 21): | | | |
| | | | | | from 2599451 to | | | |
| | | | | | 2812870. | | | |
| | | GB_BA1: | 213420 | Z99117 | Bacillus subtilis | Bacillus | 35,622 | 26-NOV-97 |
| | | BSUB0014 | | | complete genome | subtilis | | |
| | | | | | (section 14 of 21): | | | |
| | | | | | from 2599451 | | | |
| | | | | | to 2812870. | | | |
| rxa00348 | 519 | GB_PL2: | 68554 | AC007045 | Arabidopsis thaliana | Arabidopsis | 43,513 | 31-MAR-1999 |
| | | ATAC007045 | | | chromosome II BAC | thaliana | | |
| | | | | | F23M2 genomic sequence, | | | |
| | | | | | complete sequence. | | | |
| | | GB_PL2: | 5777 | AJ133743 | Arabidopsis thaliana | Arabidopsis | 38,247 | 18-Jun-99 |
| | | ATH133743 | | | ttg1 gene. | thaliana | | |
| | | GB_PL1: | 74589 | AB010068 | Arabidopsis thaliana | Arabidopsis | 34,387 | 20-Nov-99 |
| | | AB010068 | | | genomic DNA, | thaliana | | |
| | | | | | chromosome 5, | | | |
| | | | | | TAC clone: K18P6, | | | |
| | | | | | complete sequence. | | | |
| rxa00350 | 450 | GB_PL1: | 54719 | Z70678 | S. cerevisiae | Saccharomyces | 35,347 | 16-MAY-1997 |
| | | SCXV55KB | | | chromosome XV | cerevisiae | | |
| | | | | | DNA, 54.7 kb region. | | | |
| | | GB_PL1: | 1732 | Z74960 | S. cerevisiae | Saccharomyces | 35,347 | 11-Aug-97 |
| | | SCYOR052C | | | chromosome XV | cerevisiae | | |
| | | | | | reading frame | | | |
| | | | | | ORF YOR052c. | | | |
| | | GB_BA1: | 2600 | AJ006703 | Pseudanabaena sp | Pseudanabaena | 37,978 | 19-Jan-99 |
| | | PSE6703 | | | gene encoding | sp. | | |
| | | | | | for glutamine | | | |
| | | | | | synthetase. | | | |
| rxa00363 | 843 | GB_VI: | 9215 | M27470 | Simian | Simian | 35,379 | 13-MAR-1997 |
| | | SIVMNDGB1 | | | immunodeficiency | immunodeficiency | | |
| | | | | | virus, complete genome. | virus | | |
| | | GB_OM: | 1198 | U35642 | Bos taurus alpha 1- | Bos taurus | 40,131 | 5-Sep-96 |
| | | BTU35642 | | | microglobulin/bikunin | | | |
| | | | | | mRNA, complete cds. | | | |
| | | GB_PL1: | 1633 | AJ011518 | Malus domestica acc | Malus | 40,343 | 23-OCT-1998 |
| | | MDO011518 | | | synthase gene, | domestica | | |
| | | | | | exons 1-4, partial. | | | |
| rxa00400 | 1002 | GB_HTG2: | 203407 | AC006174 | Homo sapiens | Homo sapiens | 38,320 | 09-DEC-1998 |
| | | AC006174 | | | chromosome 10 clone | | | |
| | | | | | CIT987SK-1057L21 | | | |
| | | | | | map 10q25, | | | |
| | | | | | *** SEQUENCING | | | |
| | | | | | IN PROGRESS ***, | | | |
| | | | | | 6 unordered pieces. | | | |
| | | GB_HTG2: | 203407 | AC006174 | Homo sapiens | Homo sapiens | 38,320 | 09-DEC-1998 |
| | | AC006174 | | | chromosome 10 clone | | | |
| | | | | | CIT987SK-1057L21 | | | |
| | | | | | map 10q25, | | | |
| | | | | | ***SEQUENCING | | | |
| | | | | | IN PROGRESS ***, | | | |
| | | | | | 6 unordered pieces. | | | |
| | | GB_HTG2: | 203407 | AC006174 | Homo sapiens | Homo sapiens | 37,693 | 09-DEC-1998 |
| | | AC006174 | | | chromosome 10 clone | | | |
| | | | | | CIT987SK-1057L21 | | | |
| | | | | | map 10q25, | | | |
| | | | | | ***SEQUENCING | | | |
| | | | | | IN PROGRESS ***, | | | |
| | | | | | 6 unordered pieces. | | | |
| rxa00464 | | | | | | | | |
| rxa00494 | 420 | GB_BA2: | 40897 | AF004835 | Brevibacillus brevis | Brevibacillus | 40,500 | 18-NOV-97 |
| | | AF004835 | | | tyrocidine | brevis | | |
| | | | | | biosynthesis operon, | | | |
| | | | | | tyrocidine synthetase | | | |
| | | | | | 1 (tycA), tyrocidine | | | |
| | | | | | synthetase 2 (tycB), | | | |
| | | | | | tyrocidine synthetase | | | |
| | | | | | 3 (tycC), putative | | | |
| | | | | | ABC-transporter | | | |
| | | | | | TycD (tycD), putative | | | |
| | | | | | ABC-transporter TycE | | | |
| | | | | | (tycE) and putative | | | |
| | | | | | thioesterase GrsT | | | |
| | | | | | homolog (tycF) genes, | | | |
| | | | | | complete cds. | | | |
| | | GB_PR3: | 78011 | AL008712 | Human DNA sequence | Homo sapiens | 35,749 | 23-Nov-99 |
| | | HS84F12 | | | from PAC 84F12 on | | | |
| | | | | | chromosome Xq25-Xq26.3. | | | |
| | | | | | Contains glypican-3 | | | |
| | | | | | precursor (intestinal | | | |
| | | | | | protein OCI-5) | | | |
| | | | | | (GTR2-2), ESTs and | | | |
| | | | | | CA repeat. | | | |
| | | GB_PR3: | 37005 | AC005239 | Homo sapiens chromosome | Homo sapiens | 33,663 | 3-Jul-98 |
| | | AC005239 | | | 19, cosmid F23149, | | | |
| | | | | | complete sequence. | | | |
| rxa00516 | 843 | GB_PR3: | 206880 | AF020503 | Homo sapiens FRA3B | Homo sapiens | 40,503 | 23-Jan-98 |
| | | AF020503 | | | common fragile | | | |
| | | | | | region, diadenosine | | | |
| | | | | | triphosphate hydrolase | | | |
| | | | | | (FHIT) gene, exon 5. | | | |
| | | GB_HTG2: | 210344 | AC007100 | Homo sapiens clone | Homo sapiens | 37,226 | 7-Apr-99 |
| | | AC007100 | | | NH0462D13, | | | |
| | | | | | *** SEQUENCING | | | |
| | | | | | IN PROGRESS ***, | | | |
| | | | | | 5 unordered pieces. | | | |
| | | GB_HTG2: | 210344 | AC007100 | Homo sapiens clone | Homo sapiens | 37,226 | 7-Apr-99 |
| | | AC007100 | | | NH0462D13, | | | |
| | | | | | *** SEQUENCING | | | |
| | | | | | IN PROGRESS ***, | | | |
| | | | | | 5 unordered pieces. | | | |
| rxa00551 | 594 | GB_EST27: | 607 | AI405761 | GH25883.5prime GH | Drosophila | 40,481 | 8-Feb-99 |
| | | AI405761 | | | Drosophila melanogaster | melanogaster | | |
| | | | | | head pOT2 | | | |
| | | | | | Drosophila melanogaster | | | |
| | | | | | cDNA clone GH25883 | | | |
| | | | | | 5prime, mRNA sequence. | | | |
| | | GB_EST27: | 607 | AI405774 | GH25902.5prime GH | Drosophila | 40,481 | 8-Feb-99 |
| | | AI405774 | | | Drosophila melanogaster | melanogaster | | |
| | | | | | head pOT2 | | | |
| | | | | | Drosophila melanogaster | | | |
| | | | | | cDNA clone GH25902 | | | |
| | | | | | 5prime, mRNA sequence. | | | |
| | | GB_EST22: | 674 | AI063444 | GH03263.5prime GH | Drosophila | 40,437 | 24-Nov-98 |
| | | AI063444 | | | Drosophila melanogaster | melanogaster | | |
| | | | | | head pOT2 | | | |
| | | | | | Drosophila melanogaster | | | |
| | | | | | cDNA clone GH03263 | | | |
| | | | | | 5prime, | | | |
| | | | | | mRNA sequence. | | | |
| rxa00583 | 861 | GB_BA1: | 2570 | L07603 | Corynebacterium | Corynebacterium | 97,310 | 26-Apr-93 |
| | | CORAHPS | | | glutamicum | glutamicum | | |
| | | | | | 3-deoxy-D- | | | |
| | | | | | arabinoheptulosonate- | | | |
| | | | | | 7-phosphate | | | |
| | | | | | synthase gene, | | | |
| | | | | | complete cds. | | | |
| | | GB_BA1: | 67200 | AL021897 | Mycobacterium | Mycobacterium | 58,769 | 24-Jun-99 |
| | | MTV017 | | | tuberculosis | tuberculosis | | |
| | | | | | H37Rv complete | | | |
| | | | | | genome; segment | | | |
| | | | | | 48/162. | | | |
| | | GB_IN1: | 849 | X68555 | A. californica | Aplysia | 41,417 | 30-Jun-98 |
| | | ACKRPA | | | KRP-A gene. | californica | | |
| rxa00592 | 582 | GB_IN2: | 62091 | AC005467 | Drosophila | Drosophila | 33,565 | 12-DEC-1998 |
| | | AC005467 | | | melanogaster, | melanogaster | | |
| | | | | | chromosome 2R, | | | |
| | | | | | region 48C1-48C2, | | | |
| | | | | | P1 clone | | | |
| | | | | | DS00568, complete | | | |
| | | | | | sequence. | | | |
| | | GB_IN2: | 62091 | AC005467 | Drosophila | Drosophila | 35,893 | 12-DEC-1998 |
| | | AC005467 | | | melanogaster, | melanogaster | | |
| | | | | | chromosome 2R, | | | |
| | | | | | region 48C1-48C2, | | | |
| | | | | | P1 clone DS00568, | | | |
| | | | | | complete sequence. | | | |
| rxa00593 | 471 | GB_BA1: | 121125 | AL022121 | Mycobacterium | Mycobacterium | 33,761 | 24-Jun-99 |
| | | MTV025 | | | tuberculosis | tuberculosis | | |
| | | | | | H37Rv complete | | | |
| | | | | | genome; | | | |
| | | | | | segment 155/162. | | | |
| | | GB_BA1: | 37770 | L01263 | M. leprae genomic dna | Mycobacterium | 35,065 | 14-Jun-96 |
| | | MSGB577CO | | | sequence, cosmid b577. | leprae | | |
| | | S | | | | | | |
| | | GB_BA2: | 2366 | AF114720 | Xanthomonas | Xanthomonas | 37,768 | 1-Feb-99 |
| | | AF114720 | | | campestris pv. | campestris | | |
| | | | | | vesicatoria | pv. | | |
| | | | | | avirulence protein | vesicatoria | | |
| | | | | | AvrBs2 (avrBs2) gene, | | | |
| | | | | | complete cds. | | | |
| rxa00603 | 576 | GB_BA1: | 4357 | X78346 | R. capsulatus (B10S) | Rhodobacter | 34,867 | 08-DEC-1995 |
| | | RCPUTRA | | | putR and | capsulatus | | |
| | | | | | putA genes. | | | |
| | | GB_GSS10: | 474 | AQ227452 | HS_2015_B2_B07_MR CIT | Homo sapiens | 35,337 | 26-Sep-98 |
| | | AQ227452 | | | Approved Human | | | |
| | | | | | Genomic Sperm | | | |
| | | | | | Library D Homo | | | |
| | | | | | sapiens genomic | | | |
| | | | | | clone Plate = | | | |
| | | | | | 2015 Col = 14 | | | |
| | | | | | Row = D, | | | |
| | | | | | genomic survey | | | |
| | | | | | sequence. | | | |
| | | GB_GSS3: | 251 | B60643 | CIT-HSP-2015D14.TRB | Homo sapiens | 39,200 | 21-Jun-98 |
| | | B60643 | | | CIT-HSP | | | |
| | | | | | Homo sapiens | | | |
| | | | | | genomic clone | | | |
| | | | | | 2015D14, genomic | | | |
| | | | | | survey sequence. | | | |
| rxa00609 | 558 | GB_HTG3: | 105005 | AC009346 | Drosophila | Drosophila | 31,261 | 27-Aug-99 |
| | | AC009346 | | | melanogaster | melanogaster | | |
| | | | | | chromosome 3 clone | | | |
| | | | | | BACR03P13 (D672) | | | |
| | | | | | RPCI-98 03.P.13 map | | | |
| | | | | | 83A-83B strain | | | |
| | | | | | y; cn bw sp, | | | |
| | | | | | *** SEQUENCING | | | |
| | | | | | IN PROGRESS***, | | | |
| | | | | | 83 unordered pieces. | | | |
| | | GB_HTG3: | 105005 | AC009346 | Drosophila | Drosophila | 31,261 | 27-Aug-99 |
| | | AC009346 | | | melanogaster | melanogaster | | |
| | | | | | chromosome 3 clone | | | |
| | | | | | BACR03P13 (D672) | | | |
| | | | | | RPCI-98 03.P.13 map | | | |
| | | | | | 83A-83B strain | | | |
| | | | | | y; cn bw sp, | | | |
| | | | | | *** SEQUENCING | | | |
| | | | | | IN PROGRESS***, | | | |
| | | | | | 83 unordered pieces. | | | |
| | | GB_HTG3: | 105005 | AC009346 | Drosophila | Drosophila | 30,072 | 27-Aug-99 |
| | | AC009346 | | | melanogaster | melanogaster | | |
| | | | | | chromosome 3 clone | | | |
| | | | | | BACR03P13 (D672) | | | |
| | | | | | RPCI-98 03.P.13 map | | | |
| | | | | | 83A-83B strain | | | |
| | | | | | y; cn bw sp, | | | |
| | | | | | *** SEQUENCING | | | |
| | | | | | IN PROGRESS***, | | | |
| | | | | | 83 unordered pieces. | | | |
| rxa00630 | 828 | GB_BA1: | 36850 | Z80226 | Mycobacterium | Mycobacterium | 60,870 | 17-Jun-98 |
| | | MTCY369 | | | tuberculosis | tuberculosis | | |
| | | | | | H37Rv complete | | | |
| | | | | | genome; | | | |
| | | | | | segment 36/162. | | | |
| | | GB_BA1: | 15560 | AL020958 | Streptomyces | Streptomyces | 48,474 | 10-DEC-1997 |
| | | SC4H8 | | | coelicolor | coelicolor | | |
| | | | | | cosmid 4H8. | | | |
| | | GB_BA1: | 37218 | Z77162 | Mycobacterium | Mycobacterium | 46,537 | 17-Jun-98 |
| | | MTCY20G9 | | | tuberculosis | tuberculosis | | |
| | | | | | H37Rv complete | | | |
| | | | | | genome; | | | |
| | | | | | segment 25/162. | | | |
| rxa00651 | 1455 | GB_PR2: | 100000 | AP000165 | Homo sapiens | Homo sapiens | 35,685 | 20-Nov-99 |
| | | AP000165 | | | genomic DNA, | | | |
| | | | | | chromosome 21q22.1, | | | |
| | | | | | D21S226-AML region, | | | |
| | | | | | clone B2344F14-f50E8, | | | |
| | | | | | segment 1/9, | | | |
| | | | | | complete sequence. | | | |
| | | GB_RO: | 132297 | AC005835 | Mus musculus clone | Mus musculus | 37,851 | 21-OCT-1998 |
| | | AC005835 | | | UWGC: mbac82 | | | |
| | | | | | from 14D1-D2 | | | |
| | | | | | (T-Cell Receptor | | | |
| | | | | | Alpha Locus), | | | |
| | | | | | complete sequence. | | | |
| | | GB_PR2: | 100000 | AP000165 | Homo sapiens | Homo sapiens | 35,610 | 20-Nov-99 |
| | | AP000165 | | | genomic DNA, | | | |
| | | | | | chromosome 21q22.1, | | | |
| | | | | | D21S226-AML | | | |
| | | | | | region, clone | | | |
| | | | | | B2344F14-f50E8, | | | |
| | | | | | segment 1/9, | | | |
| | | | | | complete sequence. | | | |
| rxa00655 | 762 | GB_PR3: | 113803 | AC004460 | Homo sapiens PAC | Homo sapiens | 38,606 | 24-MAR-1998 |
| | | AC004460 | | | clone DJ1086D14, | | | |
| | | | | | complete sequence. | | | |
| | | GB_PL1: | 7707 | M87526 | Chlamydomonas | Chlamydomonas | 39,067 | 27-Apr-93 |
| | | CRERSP4A | | | reinhardtii | reinhardtii | | |
| | | | | | flagellar radial | | | |
| | | | | | spoke protein | | | |
| | | | | | (RSP4) and RSP6) | | | |
| | | | | | genes, complete cds. | | | |
| | | GB_EST38: | 517 | AW041495 | EST284359 tomato mixed | Lycopersicon | 38,760 | 18-OCT-1999 |
| | | AW041495 | | | elicitor, BTI | esculentum | | |
| | | | | | Lycopersicon esculentum | | | |
| | | | | | cDNA clone cLET14F2, | | | |
| | | | | | mRNA sequence. | | | |
| rxa00813 | 1254 | GB_BA1: | 1009 | D38230 | Mycobacterium bovis | Mycobacterium | 40,956 | 8-Feb-99 |
| | | MSGMPB70B | | | DNA for MPB70, | bovis | | |
| | | | | | complete cds, | | | |
| | | | | | strain: BCG Tokyo. | | | |
| | | GB_BA1: | 39991 | Z74024 | Mycobacterium | Mycobacterium | 41,447 | 19-Jun-98 |
| | | MTCY274 | | | tuberculosis H37Rv | tuberculosis | | |
| | | | | | complete genome; | | | |
| | | | | | segment 126/162. | | | |
| | | GB_BA1: | 1009 | D38229 | Mycobacterium bovis | Mycobacterium | 40,956 | 8-Feb-99 |
| | | MSGMPB70A | | | DNA for MPB70, | bovis | | |
| | | | | | complete cds, strain: | | | |
| | | | | | BCG Pasteur. | | | |
| rxa00822 | 804 | GB_BA1: | 121125 | AL022121 | Mycobacterium | Mycobacterium | 64,925 | 24-Jun-99 |
| | | MTV025 | | | tuberculosis H37Rv | tuberculosis | | |
| | | | | | complete genome; | | | |
| | | | | | segment 155/162. | | | |
| | | GB_EST35: | 646 | AI857185 | 603007G10.x1 603 - | Zea mays | 40,206 | 16-Jul-99 |
| | | AI857185 | | | stressed root | | | |
| | | | | | cDNA library from | | | |
| | | | | | Wang/Bohnert lab | | | |
| | | | | | Zea mays cDNA, | | | |
| | | | | | mRNA sequence. | | | |
| | | GB_PR3: | 138849 | 297181 | Homo sapiens DNA | Homo sapiens | 37,633 | 23-NOV-99 |
| | | HS95C20 | | | sequence from PAC | | | |
| | | | | | 95C20 on chromosome | | | |
| | | | | | Xp11.3-11.4. | | | |
| | | | | | Contains STSs and | | | |
| | | | | | the DXS7 locus | | | |
| | | | | | with GT and GTG repeat | | | |
| | | | | | polymorphisms, | | | |
| | | | | | complete sequence. | | | |
| rxa00848 | 2043 | GB_BA1: | 34331 | Z95584 | Mycobacterium | Mycobacterium | 63,215 | 17-Jun-98 |
| | | MTCI65 | | | tuberculosis H37Rv | tuberculosis | | |
| | | | | | complete genome; | | | |
| | | | | | segment 50/162. | | | |
| | | GB_BA1: | 40056 | AD000020 | Mycobacterium | Mycobacterium | 47,938 | 10-DEC-1996 |
| | | MSGY348 | | | tuberculosis sequence | tuberculosis | | |
| | | | | | from clone y348. | | | |
| | | GB_HTG3: | 207341 | AC008608 | Homo sapiens | Homo sapiens | 43,001 | 3-Aug-99 |
| | | AC008608 | | | chromosome 5 clone | | | |
| | | | | | CIT978SKB_13I20, | | | |
| | | | | | *** SEQUENCING IN | | | |
| rxa00849 | 444 | GB_HTG4: | 216524 | AC007305 | Mus musculus, | Mus musculus | 38,979 | 23-OCT-1999 |
| | | AC007305 | | | *** SEQUENCING IN | | | |
| | | | | | PROGRESS ***, | | | |
| | | | | | 10 unordered pieces. | | | |
| | | GB_HTG4: | 216524 | AC007305 | Mus musculus, | Mus musculus | 38,979 | 23-OCT-1999 |
| | | AC007305 | | | *** SEQUENCING IN | | | |
| | | | | | PROGRESS ***, | | | |
| | | | | | 10 unordered pieces. | | | |
| | | GB_HTG4: | 216524 | AC007305 | Mus musculus, | Mus musculus | 36,636 | 23-OCT-1999 |
| | | AC007305 | | | *** SEQUENCING IN | | | |
| | | | | | PROGRESS ***, | | | |
| | | | | | 10 unordered pieces. | | | |
| rxa00885 | 1149 | GB_EST36: | 300 | AV178106 | AV178106 Yuji Kohara | Caenorhabditis | 39,057 | 21-Jul-99 |
| | | AV178106 | | | unpublished cDNA: | elegans | | |
| | | | | | Strain N2 hermaphrodite | | | |
| | | | | | embryo | | | |
| | | | | | Caenorhabditis elegans | | | |
| | | | | | cDNA clone yk538b7 | | | |
| | | | | | 3′, mRNA sequence. | | | |
| | | GB_EST16: | 300 | C30090 | C30090 Yuji Kohara | Caenorhabditis | 38,000 | 18-OCT-1999 |
| | | C30090 | | | unpublished cDNA: | elegans | | |
| | | | | | Strain N2 hermaphrodite | | | |
| | | | | | embryo | | | |
| | | | | | Caenorhabditis elegans | | | |
| | | | | | cDNA clone yk236d2 | | | |
| | | | | | 3′, mRNA sequence. | | | |
| | | GB_IN1: | 32679 | Z68220 | Caenorhabditis elegans | Caenorhabditis | 36,067 | 2-Sep-99 |
| | | CET20D3 | | | cosmid T20D3, | elegans | | |
| | | | | | complete sequence. | | | |
| rxa00894 | 1251 | GB_EST20: | 281 | AA890839 | TENS0689 T. cruzi | Trypanosoma | 39,779 | 29-OCT-1998 |
| | | AA890839 | | | epimastigote | cruzi | | |
| | | | | | normalized cDNA | | | |
| | | | | | Library | | | |
| | | | | | Trypanosoma cruzi | | | |
| | | | | | cDNA clone 689 5′, | | | |
| | | | | | mRNA sequence. | | | |
| | | GB_EST20: | 284 | AA890838 | TENS0687 T. cruzi | Trypanosoma | 39,674 | 29-OCT-1998 |
| | | AA890838 | | | epimastigote | cruzi | | |
| | | | | | normalized cDNA | | | |
| | | | | | Library | | | |
| | | | | | Trypanosoma cruzi | | | |
| | | | | | cDNA clone 687 | | | |
| | | | | | 5′, mRNA sequence. | | | |
| | | GB_RO: | 1709 | X97192 | R. norvegicus MAFA | Rattus | 36,989 | 17-Apr-96 |
| | | RNMAFAEX2 | | | gene, exon2. | norvegicus | | |
| rxa00947 | 459 | GB_EST6: | 420 | W04640 | zb93b03.s1 Soares_{—} | Homo sapiens | 43,519 | 23-Apr-96 |
| | | W04640 | | | parathyroid_tumor_{—} | | | |
| | | | | | NbHPA Homo sapiens | | | |
| | | | | | cDNA clone IMAGE: | | | |
| | | | | | 320333 3′, | | | |
| | | | | | mRNA sequence. | | | |
| | | GB_EST6: | 420 | W04640 | zb93b03.s1 Soares_{—} | Homo sapiens | 37,725 | 23-Apr-96 |
| | | W04640 | | | parathyroid_{—} | | | |
| | | | | | tumor_NbHPA | | | |
| | | | | | Homo sapiens cDNA | | | |
| | | | | | clone IMAGE: | | | |
| | | | | | 320333 3′, | | | |
| | | | | | mRNA sequence. | | | |
| rxa01001 | | | | | | | | |
| rxa01065 | 1038 | GB_BA1: | 27548 | Z95208 | Mycobacterium | Mycobacterium | 38,949 | 17-Jun-98 |
| | | MTCY27 | | | tuberculosis H37Rv | tuberculosis | | |
| | | | | | complete genome; | | | |
| | | | | | segment 104/162. | | | |
| | | GB_BA2: | 35209 | AF065159 | Bradyrhizobium | Bradyrhizobium | 46,369 | 27-OCT-1999 |
| | | AF065159 | | | japonicum putative | japonicum | | |
| | | | | | arylsulfatase | | | |
| | | | | | (arsA), putative | | | |
| | | | | | soluble lytic | | | |
| | | | | | transglycosylase | | | |
| | | | | | precursor (sltA), | | | |
| | | | | | dihydrodipicolinate | | | |
| | | | | | synthase (dapA), MscL | | | |
| | | GB_HTG2: | 297866 | AC006794 | Caenorhabditis elegans | Caenorhabditis | 34,676 | 23-Feb-99 |
| | | AC006794 | | | clone Y50D4a, | elegans | | |
| | | | | | *** SEQUENCING | | | |
| | | | | | IN PROGRESS***, | | | |
| | | | | | 29 unordered pieces. | | | |
| rxa01110 | 696 | GB_HTG7: | 204901 | AC009530 | Homo sapiens | Homo sapiens | 36,364 | 08-DEC-1999 |
| | | AC009530 | | | chromosome 7, | | | |
| | | | | | *** SEQUENCING | | | |
| | | | | | IN PROGRESS ****, | | | |
| | | | | | 32 unordered pieces. | | | |
| | | GB_HTG3: | 163369 | AC009301 | Homo sapiens clone | Homo sapiens | 34,538 | 13-Aug-99 |
| | | AC009301 | | | NH0062F14, | | | |
| | | | | | *** SEQUENCING | | | |
| | | | | | IN PROGRESS ***, | | | |
| | | | | | 5 unordered pieces. | | | |
| | | GB_HTG3: | 163369 | AC009301 | Homo sapiens clone | Homo sapiens | 34,538 | 13-Aug-99 |
| | | AC009301 | | | NH0062F14, | | | |
| | | | | | *** SEQUENCING | | | |
| | | | | | IN PROGRESS ***, | | | |
| | | | | | 5 unordered pieces. | | | |
| rxa01118 | 888 | GB_BA2: | 5475 | AF003947 | Rhodococcus opacus | Rhodococcus | 55,982 | 12-MAR-1998 |
| | | AF003947 | | | succinyl CoA: | opacus | | |
| | | | | | 3-oxoadipate CoA | | | |
| | | | | | transferase | | | |
| | | | | | subunit homolog | | | |
| | | | | | (pcal′) | | | |
| | | | | | gene, partial cds, | | | |
| | | | | | protocatechuate | | | |
| | | | | | dioxygenase beta | | | |
| | | | | | subunit (pcaH), | | | |
| | | | | | protocatechuate | | | |
| | | | | | dioxygenase | | | |
| | | | | | alpha subunit | | | |
| | | | | | (pcaG), 3-carboxy- | | | |
| | | | | | cis,cis-muconate | | | |
| | | | | | cycloisomerase | | | |
| | | | | | homolog (pcaB), | | | |
| | | | | | 3-oxoadipate | | | |
| | | | | | enol-lactone | | | |
| | | | | | hydrolase/ | | | |
| | | | | | 4-carboxymuconolactone | | | |
| | | | | | decarboxylase | | | |
| | | | | | (pcaL) and PcaR | | | |
| | | | | | (pcaR) genes, | | | |
| | | | | | complete cds, | | | |
| | | | | | and 3-oxoadipyl | | | |
| | | | | | CoA thiolase | | | |
| | | | | | homolog (pcaF′) | | | |
| | | | | | gene, partial cds. | | | |
| | | GB_BA1: | 7224 | X99622 | Rhodococcus opacus | Rhodococcus | 40,000 | 24-Sep-97 |
| | | ROX99622 | | | catR, catA, catB, | opacus | | |
| | | | | | catC genes and | | | |
| | | | | | five ORFs. | | | |
| | | GB_IN1: | 42966 | U29082 | Caenorhabditis elegans | Caenorhabditis | 37,485 | 15-Jun-95 |
| | | CELC14F5 | | | cosmid C14F5. | elegans | | |
| rxa01125 | 336 | GB_EST16: | 360 | C41499 | C41499 Yuji Kohara | Caenorhabditis | 44,747 | 18-OCT-1999 |
| | | C41499 | | | unpublished cDNA: | elegans | | |
| | | | | | Strain N2 hermaphrodite | | | |
| | | | | | embryo | | | |
| | | | | | Caenorhabditis elegans | | | |
| | | | | | cDNA clone yk268f1 | | | |
| | | | | | 5′, mRNA sequence. | | | |
| | | GB_HTG2: | 195349 | AC006705 | Caenorhabditis elegans | Caenorhabditis | 42,415 | 23-Feb-99 |
| | | AC006705 | | | clone Y108G3c, | elegans | | |
| | | | | | *** SEQUENCING | | | |
| | | | | | IN PROGRESS***, | | | |
| | | | | | 2 unordered pieces. | | | |
| | | GB_IN2: | 36400 | AF067622 | Caenorhabditis elegans | Caenorhabditis | 42,415 | 27-MAY-1999 |
| | | CELF33E11 | | | cosmid F33E11. | elegans | | |
| rxa01211 | 1380 | GB_EST28: | 503 | AI520492 | LD40669.3prime | Drosophila | 40,726 | 16-MAR-1999 |
| | | AI520492 | | | LD Drosophila | melanogaster | | |
| | | | | | melanogaster | | | |
| | | | | | embryo pOT2 | | | |
| | | | | | Drosophila | | | |
| | | | | | melanogaster | | | |
| | | | | | cDNA clone LD40669 | | | |
| | | | | | 3prime, | | | |
| | | | | | mRNA sequence. | | | |
| | | GB_EST27: | 551 | AI403753 | GH23256.3prime GH | Drosophila | 41,316 | 8-Feb-99 |
| | | AI403753 | | | Drosophila | melanogaster | | |
| | | | | | melanogaster | | | |
| | | | | | head pOT2 | | | |
| | | | | | Drosophila | | | |
| | | | | | melanogaster | | | |
| | | | | | cDNA clone GH23256 | | | |
| | | | | | 3prime, mRNA sequence. | | | |
| | | GB_EST19: | 493 | AA391230 | LD10605.3prime LD | Drosophila | 38,415 | 27-Nov-98 |
| | | AA391230 | | | Drosophila | melanogaster | | |
| | | | | | melanogaster embryo | | | |
| | | | | | BlueScript | | | |
| | | | | | Drosophila | | | |
| | | | | | melanogaster | | | |
| | | | | | cDNA clone LD10605 | | | |
| | | | | | 3prime, mRNA sequence. | | | |
| rxa01241 | 603 | GB_BA1: | 36033 | U00019 | Mycobacterium leprae | Mycobacterium | 58,783 | 01-MAR-1994 |
| | | U00019 | | | cosmid B2235. | leprae | | |
| | | GB_BA1: | 22781 | L78826 | Mycobacterium leprae | Mycobacterium | 58,464 | 15-Jun-96 |
| | | MSGB42CS | | | cosmid B42 | leprae | | |
| | | | | | DNA sequence. | | | |
| | | GB_HTG5: | 173897 | AC007521 | Drosophila | Drosophila | 40,137 | 17-Nov-99 |
| | | AC007521 | | | melanogaster chromosome | melanogaster | | |
| | | | | | X clone BACR49A04 | | | |
| | | | | | (D698) RPCI-98 | | | |
| | | | | | 49.A.4 map 10A2-10B2 | | | |
| | | | | | strain y; cn bw sp, | | | |
| | | | | | *** SEQUENCING IN | | | |
| | | | | | PROGRESS***, | | | |
| | | | | | 56 unordered pieces. | | | |
| rxa01248 | 529 | GB_BA1: | 338534 | U14003 | Escherichia coli | Escherichia | 40,546 | 17-Apr-96 |
| | | ECOUW93 | | | K-12 chromosomal | coli | | |
| | | | | | region from 92.8 | | | |
| | | | | | to 00.1 minutes. | | | |
| | | GB_BA1: | 137740 | D90900 | Synechocystis sp. | Synechocystis | 32,177 | 7-Feb-99 |
| | | D90900 | | | PCC6803 complete | sp. | | |
| | | | | | genome, 2/27, | | | |
| | | | | | 133860-271599. | | | |
| | | GB_BA1: | 338534 | U14003 | Escherichia coli | Escherichia | 37,044 | 17-Apr-96 |
| | | ECOUW93 | | | K-12 chromosomal | coli | | |
| | | | | | region from 92.8 | | | |
| | | | | | to 00.1 minutes. | | | |
| rxa01272 | 726 | GB_EST10: | 520 | AA181367 | zp42c11.s1 Stratagene | Homo sapiens | 41,408 | 09-MAR-1998 |
| | | AA181367 | | | muscle 937209 | | | |
| | | | | | Homo sapiens cDNA | | | |
| | | | | | clone IMAGE: 612116 | | | |
| | | | | | 3′, mRNA sequence. | | | |
| | | GB_VI: | 330742 | U42580 | Paramecium bursaria | Paramecium | 38,265 | 4-Nov-99 |
| | | PBU42580 | | | Chlorella virus 1, | bursaria | | |
| | | | | | complete genome. | Chlorella | | |
| | | | | | | virus 1 | | |
| | | GB_VI: | 236120 | AF063866 | Melanoplus sanguinipes | Melanoplus | 38,579 | 22-DEC-1998 |
| | | AF063866 | | | entomopoxvirus, | sanguinipes | | |
| | | | | | complete genome. | entomopoxvirus | | |
| rxa01368 | 435 | GB_BA2: | 783 | AF164439 | Mycobacterium | Mycobacterium | 57,477 | 4-Aug-99 |
| | | AF164439 | | | smegmatis WhmD | smegmatis | | |
| | | | | | (whmD) gene, | | | |
| | | | | | complete cds; | | | |
| | | | | | and unknown gene. | | | |
| | | GB_BA1: | 1668 | AL021840 | Mycobacterium | Mycobacterium | 37,617 | 17-Jun-98 |
| | | MTV015 | | | tuberculosis | tuberculosis | | |
| | | | | | H37Rv | | | |
| | | | | | complete genome; | | | |
| | | | | | segment 140/162. | | | |
| | | GB_BA1: | 593 | X68708 | S. griseocarneum | Streptomyces | 53,396 | 17-Jan-94 |
| | | SGWHIB | | | whiB-Stv gene. | griseocarneus | | |
| rxa01375 | 1578 | GB_BA1: | 42729 | Z92771 | Mycobacterium | Mycobacterium | 52,638 | 10-Feb-99 |
| | | MTCY71 | | | tuberculosis H37Rv | tuberculosis | | |
| | | | | | complete genome; | | | |
| | | | | | segment 141/162. | | | |
| | | GB_IN2: | 29330 | AC005935 | Leishmania major | Leishmania | 39,777 | 15-Nov-99 |
| | | AC005935 | | | chromosome 3 | major | | |
| | | | | | clone L7234 | | | |
| | | | | | strain Friedlin, | | | |
| | | | | | complete sequence. | | | |
| | | GB_IN2: | 1962 | AF005195 | Trypanosoma cruzi | Trypanosoma | 40,304 | 17-Aug-98 |
| | | AF005195 | | | paraflagellar rod | cruzi | | |
| | | | | | component Par3 (par3b) | | | |
| | | | | | mRNA, complete cds. | | | |
| rxa01418 | 369 | GB_IN2: | 29535 | U42830 | Caenorhabditis elegans | Caenorhabditis | 34,375 | 03-MAR-1998 |
| | | CELC53B7 | | | cosmid C53B7. | elegans | | |
| | | GB_IN1: | 1118 | U49449 | Caenorhabditis elegans | Caenorhabditis | 47,111 | 17-MAY-1996 |
| | | CEU49449 | | | olfactory receptor | elegans | | |
| | | | | | Odr-10 (odr-10) mRNA, | | | |
| | | | | | complete cds. | | | |
| | | GB_EST35: | 295 | AI871077 | wI70c12.x1 | Homo sapiens | 37,722 | 30-Aug-99 |
| | | AI871077 | | | NCI_CGAP_Brn25 | | | |
| | | | | | Homo sapiens | | | |
| | | | | | cDNA clone | | | |
| | | | | | IMAGE: 2430262 3′ | | | |
| | | | | | similar to gb: | | | |
| | | | | | X70683_cds1 | | | |
| | | | | | SOX-4 PROTEIN | | | |
| | | | | | (HUMAN);, | | | |
| | | | | | mRNA sequence. | | | |
| rxa01450 | 687 | GB_BA1: | 67200 | AL021897 | Mycobacterium | Mycobacterium | 60,059 | 24-Jun-99 |
| | | MTV017 | | | tuberculosis H37Rv | tuberculosis | | |
| | | | | | complete genome; | | | |
| | | | | | segment 48/162. | | | |
| | | GB_BA1: | 4972 | X79027 | M. ammoniaphilum | Microbacterium | 39,912 | 20-Nov-96 |
| | | MAMAMIRM | | | genes mamIR | ammoniaphilum | | |
| | | | | | and mamIM. | | | |
| | | GB_HTG3: | 46469 | AC009121 | Homo sapiens | Homo sapiens | 55,507 | 3-Aug-99 |
| | | AC009121 | | | chromosome 16 clone | | | |
| | | | | | RPCI-11_485G7, | | | |
| | | | | | *** SEQUENCING IN | | | |
| | | | | | PROGRESS ***, | | | |
| | | | | | 32 unordered pieces. | | | |
| rxa01451 | 690 | GB_BA1: | 67200 | AL021897 | Mycobacterium | Mycobacterium | 63,516 | 24-Jun-99 |
| | | MTV017 | | | tuberculosis H37Rv | tuberculosis | | |
| | | | | | complete genome; | | | |
| | | | | | segment 48/162. | | | |
| | | GB_BA1: | 4972 | X79027 | M. ammoniaphilum | Microbacterium | 37,113 | 20-Nov-96 |
| | | MAMAMIRM | | | genes mamIR | ammoniaphilum | | |
| | | | | | and mamIM. | | | |
| | | GB_BA1: | 34714 | AL049491 | Mycobacterium leprae | Mycobacterium | 36,324 | 27-Aug-99 |
| | | MLCB1222 | | | cosmid B1222. | leprae | | |
| rxa01500 | 567 | GB_IN1: | 29688 | Z46791 | Caenorhabditis | Caenorhabditis | 36,298 | 2-Sep-99 |
| | | CEC09G5 | | | elegans | elegans | | |
| | | | | | cosmid C09G5, | | | |
| | | | | | complete sequence. | | | |
| | | GB_GSS9: | 390 | AQ096256 | HS_3037_A1_F11_MF CIT | Homo sapiens | 46,316 | 27-Aug-98 |
| | | AQ096256 | | | Approved Human | | | |
| | | | | | Genomic Sperm | | | |
| | | | | | Library D Homo | | | |
| | | | | | sapiens genomic | | | |
| | | | | | clone Plate = 3037 | | | |
| | | | | | Col = 21 | | | |
| | | | | | Row = K, | | | |
| | | | | | genomic | | | |
| | | | | | survey sequence. | | | |
| | | GB_HTG1: | 1301 | AL035456 | Homo sapiens | Homo sapiens | 39,388 | 23-Nov-99 |
| | | HS1099D15 | | | chromosome 20 clone | | | |
| | | | | | RP5-1099D15, | | | |
| | | | | | *** SEQUENCING IN | | | |
| | | | | | PROGRESS ***, | | | |
| | | | | | in unordered pieces. | | | |
| rxa01537 | 774 | GB_RO: | 2354 | X64589 | R. norvegicus mRNA | Rattus | 40,584 | 29-MAR-1994 |
| | | RNCYCBMR | | | for cyclin B. | norvegicus | | |
| | | GB_RO: | 1465 | L11995 | Rattus norvegicus | Rattus | 40,584 | 3-Feb-98 |
| | | RATCYCLINB | | | cyclin B mRNA, | norvegicus | | |
| | | | | | complete cds. | | | |
| | | GB_RO: | 1902 | X60768 | Rat mRNA for cyclin B. | Rattus | 40,530 | 15-Aug-96 |
| | | RNCYCLNB | | | | norvegicus | | |
| rxa01573 | 2205 | GB_HTG4: | 40524 | AC011317 | Homo sapiens | Homo sapiens | 34,814 | 21-OCT-1999 |
| | | AC011317 | | | chromosome 3 seeders | | | |
| | | | | | clone RPCI11-103G8, | | | |
| | | | | | ***SEQUENCING | | | |
| | | | | | IN PROGRESS ***, | | | |
| | | | | | 31 unordered pieces. | | | |
| | | GB_HTG4: | 40524 | AC011317 | Homo sapiens | Homo sapiens | 34,814 | 21-OCT-1999 |
| | | AC011317 | | | chromosome 3 seeders | | | |
| | | | | | clone RPCI1 1-103G8, | | | |
| | | | | | ***SEQUENCING IN | | | |
| | | | | | PROGRESS ***, | | | |
| | | | | | 31 unordered pieces. | | | |
| | | GB_IN1: | 24323 | AF039038 | Caenorhabditis elegans | Caenorhabditis | 38,899 | 1-Jan-98 |
| | | CELK06A5 | | | cosmid K06A5. | elegans | | |
| rxa01655 | 1482 | GB_GSS15: | 460 | AQ624398 | HS_2106_B2_C03_T7C CIT | Homo sapiens | 36,449 | 16-Jun-99 |
| | | AQ624398 | | | Approved Human | | | |
| | | | | | Genomic Sperm Library D | | | |
| | | | | | Homo sapiens | | | |
| | | | | | genomic clone | | | |
| | | | | | Plate = 2106 | | | |
| | | | | | Col = 6 | | | |
| | | | | | Row = F, genomic | | | |
| | | | | | survey sequence. | | | |
| | | GB_BA1: | 36734 | AL049497 | Streptomyces coelicolor | Streptomyces | 39,098 | 24-MAR-1999 |
| | | SC6G10 | | | cosmid 6G10. | coelicolor | | |
| | | GB_BA1: | 38859 | AL022602 | Mycobacterium leprae | Mycobacterium | 39,891 | 27-Aug-99 |
| | | MLCB268 | | | cosmid B268. | leprae | | |
| rxa01687 | | | | | | | | |
| rxa01759 | 885 | GB_OV: | 16201 | U11880 | Petromyzon marinus | Mitochondrion | 36,977 | 24-Sep-96 |
| | | PMU11880 | | | mitochondrion, | Petromyzon | | |
| | | | | | complete genome. | marinus | | |
| | | GB_STS: | 605 | G39160 | Z13915 Zebrafish AB | Danio rerio | 36,093 | 30-Jul-98 |
| | | G39160 | | | Danio rerio STS | | | |
| | | | | | genomic, sequence | | | |
| | | | | | tagged site. | | | |
| | | GB_STS: | 605 | G39160 | Z13915 Zebrafish AB | Danio rerio | 36,093 | 30-Jul-98 |
| | | G39160 | | | Danio rerio STS | | | |
| | | | | | genomic, sequence | | | |
| | | | | | tagged site. | | | |
| rxa01763 | 588 | GB_GSS4: | 454 | AQ701186 | HS_2129_A2_D04_T7C | Homo sapiens | 40,000 | 7-Jul-99 |
| | | AQ701186 | | | CIT Approved | | | |
| | | | | | Human Genomic Sperm | | | |
| | | | | | Library D | | | |
| | | | | | Homo sapiens genomic | | | |
| | | | | | clone Plate = | | | |
| | | | | | 2129 Col = 8 | | | |
| | | | | | Row = G, | | | |
| | | | | | genomic survey | | | |
| | | | | | sequence. | | | |
| | | GB_BA1: | 5363 | D28859 | Enterococcus faecalis | Enterococcus | 37,117 | 7-Feb-99 |
| | | ENEPPD1 | | | Plasmid pPD1 | faecalis | | |
| | | | | | DNA for iPD1, TraB, | | | |
| | | | | | TraA, ORF1 and | | | |
| | | | | | TraC, complete cds. | | | |
| | | GB_BA1: | 8526 | D78016 | Enterococcus faecalis | Enterococcus | 35,788 | 5-Feb-99 |
| | | ENEPPD1A | | | Plasmid pPD1 genes | faecalis | | |
| | | | | | for REPB, REPA, TRAC, | | | |
| | | | | | TRAB, TRAA, iPD1, | | | |
| | | | | | TRAE, TRAF, complete | | | |
| | | | | | cds and partial cds. | | | |
| rxa01826 | 2061 | GB_BA1: | 37821 | Z70722 | Mycobacterium leprae | Mycobacterium | 37,524 | 29-Aug-97 |
| | | MLCB1770 | | | cosmid B1770. | leprae | | |
| | | GB_BA1: | 35824 | AL079308 | Streptomyces coelicolor | Streptomyces | 51,185 | 15-Jun-99 |
| | | SCH69 | | | cosmid H69. | coelicolor | | |
| | | GB_BA1: | 33779 | AL096822 | Streptomyces coelicolor | Streptomyces | 38,775 | 8-Jul-99 |
| | | SCGD3 | | | cosmid GD3. | coelicolor | | |
| rxa01827 | 1530 | GB_BA1: | 39160 | Z80233 | Mycobacterium | Mycobacterium | 37,815 | 17-Jun-98 |
| | | MTCY10H4 | | | tuberculosis H37Rv | tuberculosis | | |
| | | | | | complete genome; | | | |
| | | | | | segment 2/162. | | | |
| | | GB_BA1: | 2711 | AB016932 | Streptomyces | Streptomyces | 42,543 | 11-Nov-98 |
| | | AB016932 | | | coelicolor gene for | coelicolor | | |
| | | | | | protein serine/ | | | |
| | | | | | threonine kinase, | | | |
| | | | | | complete cds. | | | |
| | | GB_RO: | 2201 | AF145705 | Mus musculus T2K | Mus musculus | 40,438 | 2-Jun-99 |
| | | AF145705 | | | protein kinase | | | |
| | | | | | homolog mRNA, | | | |
| | | | | | complete cds. | | | |
| rxa01830 | 1476 | GB_PR2: | 156854 | U82672 | Human chromosome X | Homo sapiens | 36,389 | 12-MAY-1997 |
| | | HSU82672 | | | clone Qc15B1, | | | |
| | | | | | complete sequence. | | | |
| | | GB_BA2: | 26245 | AF087482 | Pseudomonas | Pseudomonas | 40,805 | 31-OCT-1998 |
| | | AF087482 | | | aeruginosa | aeruginosa | | |
| | | | | | cIcC and ohbH genes, | | | |
| | | | | | Lys-R type regulatory | | | |
| | | | | | protein | | | |
| | | | | | (clcR), chlorocatechol- | | | |
| | | | | | 1,2-dioxygenase | | | |
| | | | | | (clcA), chloromuconate | | | |
| | | | | | cycloisomerase | | | |
| | | | | | (clcB), dienelactone | | | |
| | | | | | hydrolase (clcD), | | | |
| | | | | | maleylacetate | | | |
| | | | | | reductase (clcE), | | | |
| | | | | | transposase (tnpA), | | | |
| | | | | | ATP-binding protein | | | |
| | | | | | (tnpB), putative | | | |
| | | | | | regulatory protein | | | |
| | | | | | (ohbR), | | | |
| | | | | | o-halobenzoate | | | |
| | | | | | dioxygenase | | | |
| | | | | | reductase (ohbA), | | | |
| | | | | | o-halobenzoate | | | |
| | | | | | dioxygenase alpha | | | |
| | | | | | subunit (ohbB), | | | |
| | | | | | o-halobenzoate | | | |
| | | | | | dioxygenase | | | |
| | | | | | beta subunit (ohbC), | | | |
| | | | | | o-halobenzoate | | | |
| | | | | | dioxygenase | | | |
| | | | | | ferredoxin (ohbD), | | | |
| | | | | | putative membrane | | | |
| | | | | | spanning protein | | | |
| | | | | | (ohbE), ATP-binding | | | |
| | | | | | protein (ohbF), | | | |
| | | | | | putative substrate | | | |
| | | | | | binding protein | | | |
| | | | | | (ohbG), and putative | | | |
| | | | | | dioxygenase genes, | | | |
| | | | | | complete cds; and | | | |
| | | | | | unknown gene. | | | |
| | | GB_PR2: | 156854 | U82672 | Human chromosome X | Homo sapiens | 36,301 | 12-MAY-1997 |
| | | HSU82672 | | | clone Qc15B1, | | | |
| | | | | | complete sequence. | | | |
| rxa01836 | 828 | GB_GSS1: | 704 | AJ227010 | Ciona intestinalis | Ciona | 33,481 | 10-MAR-1998 |
| | | CI22H2 | | | genomic fragment, | intestinalis | | |
| | | | | | clone 22H2, genomic | | | |
| | | | | | survey sequence. | | | |
| | | GB_EST18: | 461 | AA692868 | vr58h12.s1 Knowles | Mus musculus | 47,222 | 16-DEC-1997 |
| | | AA692868 | | | Solter mouse 2 cell | | | |
| | | | | | Mus musculus | | | |
| | | | | | cDNA clone IMAGE: | | | |
| | | | | | 1124903 5′, | | | |
| | | | | | mRNA sequence. | | | |
| | | GB_PR3: | 156791 | AL049594 | Human DNA sequence from | Homo sapiens | 35,504 | 23-Nov-99 |
| | | HSDJ860P4 | | | clone 860P4 on | | | |
| | | | | | chromosome 20 | | | |
| | | | | | Contains ESTs, | | | |
| | | | | | STSs, GSSs and a | | | |
| | | | | | CpG island, | | | |
| | | | | | complete sequence. | | | |
| rxa01840 | 654 | GB_BA1: | 145709 | D90914 | Synechocystis sp. | Synechocystis | 61,315 | 7-Feb-99 |
| | | D90914 | | | PCC6803 complete | sp. | | |
| | | | | | genome, 16/27, | | | |
| | | | | | 1991550-2137258. | | | |
| | | GB_EST25: | 306 | AU041657 | AU041657 Mouse | Mus musculus | 39,216 | 04-DEC-1998 |
| | | AU041657 | | | four-cell- | | | |
| | | | | | embryo cDNA | | | |
| | | | | | Mus musculus cDNA | | | |
| | | | | | clone J1007D01 | | | |
| | | | | | 3′, mRNA sequence. | | | |
| | | GB_PL2: | 474 | U82633 | Alternaria alternata | Alternaria | 45,092 | 13-Jan-97 |
| | | AAU82633 | | | Alt a I subunit | alternata | | |
| | | | | | mRNA, complete cds. | | | |
| rxa01860 | 1008 | GB_PL2: | 97789 | AC004255 | Arabidopsis thaliana | Arabidopsis | 35,939 | 16-Apr-98 |
| | | AC004255 | | | BAC T1F9 chromosome | thaliana | | |
| | | | | | 1, complete sequence. | | | |
| | | GB_BA1: | 213190 | Z99107 | Bacillus subtilis | Bacillus | 37,111 | 26-NOV-97 |
| | | BSUB0004 | | | complete genome | subtilis | | |
| | | | | | (section 4 of 21): | | | |
| | | | | | from 600701 to 813890. | | | |
| | | GB_BA1: | 20341 | D86418 | Bacillus subtilis | Bacillus | 38,352 | 7-Feb-99 |
| | | D86418 | | | genomic DNA 69-70 | subtilis | | |
| | | | | | degree region, | | | |
| | | | | | partial sequence. | | | |
| rxa01861 | 2088 | GB_HTG4: | 173517 | AC009949 | Homo sapiens chromosome | Homo sapiens | 36,544 | 29-OCT-1999 |
| | | AC009949 | | | unknown clone | | | |
| | | | | | NH0069J07, | | | |
| | | | | | WORKING DRAFT | | | |
| | | | | | SEQUENCE, in | | | |
| | | | | | unordered pieces. | | | |
| | | GB_HTG4: | 173517 | AC009949 | Homo sapiens chromosome | Homo sapiens | 36,544 | 29-OCT-1999 |
| | | AC009949 | | | unknown clone | | | |
| | | | | | NH0069J07, | | | |
| | | | | | WORKING DRAFT | | | |
| | | | | | SEQUENCE, | | | |
| | | | | | in unordered pieces. | | | |
| | | GB_HTG4: | 173517 | AC009949 | Homo sapiens chromosome | Homo sapiens | 35,676 | 29-OCT-1999 |
| | | AC009949 | | | unknown clone | | | |
| | | | | | NH0069J07, | | | |
| | | | | | WORKING DRAFT | | | |
| | | | | | SEQUENCE, | | | |
| | | | | | in unordered pieces. | | | |
| rxa01898 | 816 | GB_HTG1: | 293827 | AL021151 | Caenorhabditis elegans | Caenorhabditis | 33,250 | 1-Apr-99 |
| | | CEY48B6 | | | chromosome II | elegans | | |
| | | | | | clone Y48B6, | | | |
| | | | | | *** SEQUENCING IN | | | |
| | | | | | PROGRESS ***, | | | |
| | | | | | in unordered pieces. | | | |
| | | GB_HTG1: | 293827 | AL021151 | Caenorhabditis elegans | Caenorhabditis | 33,250 | 1-Apr-99 |
| | | CEY48B6 | | | chromosome II | elegans | | |
| | | | | | clone Y48B6, | | | |
| | | | | | *** SEQUENCING IN | | | |
| | | | | | PROGRESS ***, | | | |
| | | | | | in unordered pieces. | | | |
| | | GB_HTG1: | 110000 | Z92860 | Caenorhabditis elegans | Caenorhabditis | 34,766 | Z92860 |
| | | CEY53F4_2 | | | chromosome II | elegans | | |
| | | | | | clone Y53F4, | | | |
| | | | | | *** SEQUENCING IN | | | |
| | | | | | PROGRESS ***, | | | |
| | | | | | in unordered pieces. | | | |
| rxa01935 | 1287 | GB_PR3: | 48084 | AL080273 | Human DNA sequence from | Homo sapiens | 38,661 | 23-Nov-99 |
| | | HSBA259P1 | | | clone 259P1 on | | | |
| | | | | | chromosome 22. | | | |
| | | | | | Contains STSs, | | | |
| | | | | | GSSs, genomic | | | |
| | | | | | markers D22S1154, | | | |
| | | | | | D22S310 and D22S690, | | | |
| | | | | | and a gt repeat | | | |
| | | | | | polymorphism, | | | |
| | | | | | complete sequence. | | | |
| | | GB_BA1: | 2862 | M19019 | R. fredii host-inducible | Sinorhizobium | 37,007 | 26-Apr-93 |
| | | RHMIND | | | protein genes | fredii | | |
| | | | | | A and B, complete cds. | | | |
| | | GB_BA2: | 10894 | AE000108 | Rhizobium sp. | Rhizobium | 37,322 | 12-DEC-1997 |
| | | AE000108 | | | NGR234 plasmid | sp. NGR234 | | |
| | | | | | pNGR234a, section | | | |
| | | | | | 45 of 46 | | | |
| | | | | | of the complete | | | |
| | | | | | plasmid sequence. | | | |
| rxa02127 | 777 | GB_BA1: | 143051 | D90911 | Synechocystis sp. | Synechocystis | 35,480 | 7-Feb-99 |
| | | D90911 | | | PCC6803 complete | sp. | | |
| | | | | | genome, 13/27, | | | |
| | | | | | 1576593-1719643. | | | |
| | | GB_PR2: | 124095 | AC002477 | Human PAC clone | Homo sapiens | 35,409 | 22-Aug-97 |
| | | AC002477 | | | DJ327A19 | | | |
| | | | | | from Xq25-q26, | | | |
| | | | | | complete sequence. | | | |
| | | GB_PR2: | 124095 | AC002477 | Human PAC clone | Homo sapiens | 38,536 | 22-Aug-97 |
| | | AC002477 | | | DJ327A19 | | | |
| | | | | | from Xq25-q26, | | | |
| | | | | | complete sequence. | | | |
| rxa02210 | 687 | GB_BA1: | 2995 | AB025424 | Corynebacterium | Corynebacterium | 100,000 | 3-Apr-99 |
| | | AB025424 | | | glutamicum | glutamicum | | |
| | | | | | gene for aconitase, | | | |
| | | | | | partial cds. | | | |
| | | GB_EST15: | 490 | AA534896 | nf78e02.s1 NCI_{—} | Homo sapiens | 38,929 | 21-Aug-97 |
| | | AA534896 | | | CGAP_Co3 | | | |
| | | | | | Homo sapiens | | | |
| | | | | | cDNA clone IMAGE: | | | |
| | | | | | 926042 3′, | | | |
| | | | | | mRNA sequence. | | | |
| | | GB_BA1: | 2995 | AB025424 | Corynebacterium | Corynebacterium | 41,119 | 3-Apr-99 |
| | | AB025424 | | | glutamicum | glutamicum | | |
| | | | | | gene for aconitase, | | | |
| | | | | | partial cds. | | | |
| rxa02232 | 1650 | GB_BA1: | 13935 | Z98209 | Mycobacterium | Mycobacterium | 38,882 | 17-Jun-98 |
| | | MTCY154 | | | tuberculosis H37Rv | tuberculosis | | |
| | | | | | complete genome; | | | |
| | | | | | segment 121/162. | | | |
| | | GB_BA1: | 40221 | AD000002 | Mycobacterium | Mycobacterium | 56,593 | 03-DEC-1996 |
| | | MSGY154 | | | tuberculosis | tuberculosis | | |
| | | | | | sequence from | | | |
| | | | | | clone y154. | | | |
| | | GB_BA1: | 38400 | AL022268 | Streptomyces | Streptomyces | 55,569 | 6-Apr-98 |
| | | SC4H2 | | | coelicolor | coelicolor | | |
| | | | | | cosmid 4H2. | | | |
| rxa02270 | 744 | GB_BA1: | 217000 | AP000004 | Pyrococcus | Pyrococcus | 36,190 | 8-Feb-99 |
| | | AP000004 | | | horikoshii OT3 | horikoshii | | |
| | | | | | genomic DNA, | | | |
| | | | | | 777001-994000 | | | |
| | | | | | nt. position (4/7). | | | |
| | | GB_BA1: | 217000 | AP000004 | Pyrococcus | Pyrococcus | 36,951 | 8-Feb-99 |
| | | AP000004 | | | horikoshii OT3 | horikoshii | | |
| | | | | | genomic DNA, | | | |
| | | | | | 777001-994000 nt. | | | |
| | | | | | position (4/7). | | | |
| | | GB_HTG3: | 199233 | AC008403 | Homo sapiens | Homo sapiens | 38,420 | 3-Aug-99 |
| | | AC008403 | | | chromosome 19 clone | | | |
| | | | | | CIT-HSPC_273B12, | | | |
| | | | | | *** SEQUENCING | | | |
| | | | | | IN PROGRESS ***, | | | |
| | | | | | 82 unordered pieces. | | | |
| rxa02306 | 414 | GB_EST8: | 313 | AA011641 | zi02e11.s1 | Homo sapiens | 35,235 | 09-MAY-1997 |
| | | AA011641 | | | Soares_fetal_liver_{—} | | | |
| | | | | | spleen_1NFLS_S1 | | | |
| | | | | | Homo sapiens cDNA clone | | | |
| | | | | | IMAGE: 429644 3′, | | | |
| | | | | | mRNA sequence. | | | |
| | | GB_GSS1: | 527 | AL081678 | Arabidopsis thaliana | Arabidopsis | 40,615 | 28-Jun-99 |
| | | CNSOONAO | | | genome survey | thaliana | | |
| | | | | | sequence SP6 end of | | | |
| | | | | | BAC F3H19 of IGF | | | |
| | | | | | library from strain | | | |
| | | | | | Columbia of | | | |
| | | | | | Arabidopsis thaliana, | | | |
| | | | | | genomic | | | |
| | | | | | survey sequence. | | | |
| | | GB_EST24: | 494 | C97772 | C97772 Rice callus | Oryza sativa | 36,667 | 19-OCT-1998 |
| | | C97772 | | | Oryza sativa cDNA | | | |
| | | | | | clone C62702_6Z, | | | |
| | | | | | mRNA sequence. | | | |
| rxa02365 | 1968 | GB_BA1: | 42931 | U00016 | Mycobacterium leprae | Mycobacterium | 67,483 | 01-MAR-1994 |
| | | U00016 | | | cosmid B1937. | leprae | | |
| | | GB_BA1: | 41230 | Z81368 | Mycobacterium | Mycobacterium | 37,888 | 17-Jun-98 |
| | | MTCY253 | | | tuberculosis H37Rv | tuberculosis | | |
| | | | | | complete genome; | | | |
| | | | | | segment 106/162. | | | |
| | | GB_BA1: | 282700 | D84432 | Bacillus subtilis DNA, | Bacillus | 58,496 | 6-Feb-99 |
| | | BACJH642 | | | 283 Kb region | subtilis | | |
| | | | | | containing skin element. | | | |
| rxa02376 | 1626 | GB_BA2: | 3005 | U31230 | Corynebacterium | Corynebacterium | 97,504 | 2-Aug-96 |
| | | CGU31230 | | | glutamicum | glutamicum | | |
| | | | | | Obg protein homolog | | | |
| | | | | | gene, partial cds, | | | |
| | | | | | gamma glutamyl | | | |
| | | | | | kinase (proB) gene, | | | |
| | | | | | complete cds, and | | | |
| | | | | | (unkdh) gene, | | | |
| | | | | | complete cds. | | | |
| | | GB_BA1: | 1647 | D87915 | Streptomyces | Streptomyces | 58,013 | 7-Feb-99 |
| | | D87915 | | | coelicolor | coelicolor | | |
| | | | | | DNA for Obg, | | | |
| | | | | | complete cds. | | | |
| | | GB_BA1: | 53662 | AL021841 | Mycobacterium | Mycobacterium | 38,051 | 23-Jun-99 |
| | | MTV016 | | | tuberculosis H37Rv | tuberculosis | | |
| | | | | | complete genome; | | | |
| | | | | | segment 143/162. | | | |
| rxa02450 | 678 | GB_BA2: | 12391 | AE000654 | Helicobacter pylori | Helicobacter | 36,269 | 6-Apr-99 |
| | | AE000654 | | | 26695 section | pylori 26695 | | |
| | | | | | 132 of 134 of the | | | |
| | | | | | complete genome. | | | |
| | | GB_HTG3: | 165826 | AC009298 | Homo sapiens clone | Homo sapiens | 35,886 | 13-Aug-99 |
| | | AC009298 | | | NH0017I06, | | | |
| | | | | | *** SEQUENCING | | | |
| | | | | | IN PROGRESS ***, | | | |
| | | | | | 2 unordered pieces. | | | |
| | | GB_HTG4: | 110000 | AC010187_ | Homo sapiens | Homo sapiens | 38,939 | AC010187 |
| | | AC010187 | | | chromosome 3 seeders | | | |
| | | 2 | | | clone RPCI11-389O9, | | | |
| | | | | | ***SEQUENCING | | | |
| | | | | | IN PROGRESS ***, | | | |
| | | | | | 164 unordered pieces. | | | |
| rxa02493 | 1362 | GB_BA1: | 2339 | X93514 | C. glutamicum betP gene. | Corynebacterium | 38,346 | 8-Sep-97 |
| | | CGBETPGEN | | | | glutamicum | | |
| | | GB_BA1: | 107379 | X86780 | S. hygroscopicus gene | Streptomyces | 42,556 | 16-Aug-96 |
| | | SHGCPIR | | | cluster for polyketide | hygroscopicus | | |
| | | | | | immunosuppressant | | | |
| | | | | | rapamycin. | | | |
| | | GB_HTG2: | 138793 | AC007084 | Drosophila melanogaster | Drosophila | 35,985 | 2-Aug-99 |
| | | AC007084 | | | chromosome 2 clone | melanogaster | | |
| | | | | | BACR26A16 (D577) RPCI-98 | | | |
| | | | | | 26.A.16 map 43F-44A | | | |
| | | | | | strain y; cn bw sp, | | | |
| | | | | | *** SEQUENCING | | | |
| | | | | | IN PROGRESS***, | | | |
| | | | | | 19 unordered pieces. | | | |
| rxa02494 | 819 | GB_BA1: | 42991 | U00018 | Mycobacterium leprae | Mycobacterium | 42,105 | 01-MAR-1994 |
| | | U00018 | | | cosmid B2168. | leprae | | |
| | | GB_BA1: | 37218 | Z77162 | Mycobacterium | Mycobacterium | 64,552 | 17-Jun-98 |
| | | MTCY20G9 | | | tuberculosis H37Rv | tuberculosis | | |
| | | | | | complete genome; | | | |
| | | | | | segment 25/162. | | | |
| | | GB_BA1: | 3208 | Y13627 | Mycobacterium bovis | Mycobacterium | 64,428 | 6-Jan-98 |
| | | MBY13627 | | | BCG senX3, | bovis BCG | | |
| | | | | | regX3 genes. | | | |
| rxa02631 | 1488 | GB_EST17: | 468 | AA655226 | vq84a10.s1 Knowles | Mus musculus | 36,052 | 4-Nov-97 |
| | | AA655226 | | | Solter mouse 2 cell | | | |
| | | | | | Mus musculus cDNA clone | | | |
| | | | | | IMAGE: 1108986 5′ | | | |
| | | | | | similar to gb: | | | |
| | | | | | J03827 Y BOX | | | |
| | | | | | BINDING PROTEIN-1 | | | |
| | | | | | (HUMAN); gb: M62867 | | | |
| | | | | | Mouse Y box | | | |
| | | | | | transcription factor | | | |
| | | | | | (MOUSE);, mRNA | | | |
| | | | | | sequence. | | | |
| | | GB_GSS1: | 898 | AL101527 | Drosophila melanogaster | Drosophila | 34,449 | 26-Jul-99 |
| | | CNS012GD | | | genome survey | melanogaster | | |
| | | | | | sequence T7 end of BAC | | | |
| | | | | | BACN07L05 of DrosBAC | | | |
| | | | | | library from | | | |
| | | | | | Drosophila melanogaster | | | |
| | | | | | (fruit fly), | | | |
| | | | | | genomic survey sequence. | | | |
| | | GB_GSS3: | 1137 | B10133 | F2H22-T7 IGF | Arabidopsis | 38,011 | 14-MAY-1997 |
| | | B10133 | | | Arabidopsis thaliana | thaliana | | |
| | | | | | genomic clone F2H22, | | | |
| | | | | | genomic survey | | | |
| | | | | | sequence. | | | |
| rxa02632 | 819 | GB_BA1: | 36850 | Z80226 | Mycobacterium | Mycobacterium | 50,124 | 17-Jun-98 |
| | | MTCY369 | | | tuberculosis | tuberculosis | | |
| | | | | | H37Rv complete genome; | | | |
| | | | | | segment 36/162. | | | |
| | | GB_BA1: | 480 | S76966 | {BCG2 insert site} | Mycobacterium | 39,437 | 27-Jul-95 |
| | | S76966 | | | [Mycobacterium | tuberculosis | | |
| | | | | | tuberculosis, | | | |
| | | | | | BCG Japan, IS6110/IS986, | | | |
| | | | | | Insertion, 480 nt]. | | | |
| | | GB_PR3: | 188362 | AC005019 | Homo sapiens BAC clone | Homo sapiens | 36,763 | 27-Aug-98 |
| | | AC005019 | | | GS250A16 from 7p21-p22, | | | |
| | | | | | complete sequence. | | | |
| rxa02667 | 717 | GB_BA1: | 40806 | AD000016 | Mycobacterium | Mycobacterium | 55,742 | 10-DEC-1996 |
| | | MSGY23 | | | tuberculosis | tuberculosis | | |
| | | | | | sequence from clone y23. | | | |
| | | GB_BA1: | 8189 | AL022075 | Mycobacterium | Mycobacterium | 39,474 | 17-Jun-98 |
| | | MTV024 | | | tuberculosis H37Rv | tuberculosis | | |
| | | | | | complete genome; | | | |
| | | | | | segment 151/162. | | | |
| | | GB_BA1: | 38065 | AL035159 | Mycobacterium | Mycobacterium | 39,898 | 27-Aug-99 |
| | | MLCB1450 | | | leprae cosmid B1450. | leprae | | |
| rxa02668 | 846 | GB_HTG2: | 158262 | AC007739 | Homo sapiens clone | Homo sapiens | 38,659 | 5-Jun-99 |
| | | AC007739 | | | NH0091L03, | | | |
| | | | | | *** SEQUENCING | | | |
| | | | | | IN PROGRESS ***, | | | |
| | | | | | 4 unordered pieces. | | | |
| | | GB_HTG2: | 158262 | AC007739 | Homo sapiens clone | Homo sapiens | 38,659 | 5-Jun-99 |
| | | AC007739 | | | NH0091L03, | | | |
| | | | | | *** SEQUENCING | | | |
| | | | | | IN PROGRESS ***, | | | |
| | | | | | 4 unordered pieces. | | | |
| | | GB_EST24: | 443 | AI90741 | qd61a09.x1 Soares_{—} | Homo sapiens | 39,661 | 28-OCT-1998 |
| | | AI90741 | | | testis_NHT | | | |
| | | | | | Homo sapiens cDNA | | | |
| | | | | | clone IMAGE: | | | |
| | | | | | 1733944 3′, | | | |
| | | | | | mRNA sequence. | | | |
| rxa02669 | 1239 | GB_HTG2: | 158262 | AC007739 | Homo sapiens clone | Homo sapiens | 36,230 | 5-Jun-99 |
| | | AC007739 | | | NH0091L03, | | | |
| | | | | | *** SEQUENCING | | | |
| | | | | | IN PROGRESS ***, | | | |
| | | | | | 4 unordered pieces. | | | |
| | | GB_HTG2: | 158262 | AC007739 | Homo sapiens clone | Homo sapiens | 36,230 | 5-Jun-99 |
| | | AC007739 | | | NH0091L03, | | | |
| | | | | | *** SEQUENCING | | | |
| | | | | | IN PROGRESS ***, | | | |
| | | | | | 4 unordered pieces. | | | |
| | | GB_GSS9: | 425 | AQ128685 | HS_3026_B2_D10_MR | Homo sapiens | 36,235 | 23-Sep-98 |
| | | AQ128685 | | | CIT Approved | | | |
| | | | | | Human Genomic Sperm | | | |
| | | | | | Library D | | | |
| | | | | | Homo sapiens genomic | | | |
| | | | | | clone | | | |
| | | | | | Plate = 3026 | | | |
| | | | | | Col = 20 | | | |
| | | | | | Row = H, | | | |
| | | | | | genomic survey | | | |
| | | | | | sequence. | | | |
| rxa02698 | 492 | GB_EST18: | 398 | AA704727 | zj21f05.s1 | Homo sapiens | 40,470 | 24-DEC-1997 |
| | | AA704727 | | | Soares_fetal_{—} | | | |
| | | | | | liver_spleen_{—} | | | |
| | | | | | 1NFLS_S1 | | | |
| | | | | | Homo sapiens cDNA clone | | | |
| | | | | | IMAGE: 450945 3′, | | | |
| | | | | | mRNA sequence. | | | |
| | | GB_PR2: | 75698 | AP000228 | Homo sapiens | Homo sapiens | 42,616 | 20-Nov-99 |
| | | AP000228 | | | genomic DNA, | | | |
| | | | | | chromosome 21q21.2, | | | |
| | | | | | LL56-APP region, | | | |
| | | | | | clone: R49K20, | | | |
| | | | | | complete sequence. | | | |
| | | GB_PR2: | 100000 | AP000140 | Homo sapiens | Homo sapiens | 42,616 | 20-NOV-99 |
| | | AP000140 | | | genomic DNA, | | | |
| | | | | | chromosome 21q21.2, | | | |
| | | | | | LL56-APP region, clone | | | |
| | | | | | B2291C14-R44F3, | | | |
| | | | | | segment 5/10, | | | |
| | | | | | complete sequence. | | | |
| rxa02699 | 2271 | GB_GSS12: | 497 | AQ364540 | nbxb0061O09r CUGI | Oryza sativa | 37,903 | 3-Feb-99 |
| | | AQ364540 | | | Rice BAC Library | | | |
| | | | | | Oryza sativa genomic | | | |
| | | | | | clone nbxb0061O09r, | | | |
| | | | | | genomic | | | |
| | | | | | survey sequence. | | | |
| | | GB_PR4: | 141509 | AC006044 | Homo sapiens BAC clone | Homo sapiens | 36,360 | 18-MAR-1999 |
| | | AC006044 | | | NH0539B24 from | | | |
| | | | | | 7p15.1-p14, | | | |
| | | | | | complete sequence. | | | |
| | | GB_PR2: | 91526 | AF001552 | Homo sapiens | Homo sapiens | 35,352 | 21-Aug-97 |
| | | HSAF001552 | | | chromosome 16 BAC | | | |
| | | | | | clone CIT987SK-381E11 | | | |
| | | | | | complete sequence. | | | |
| rxa02724 | 967 | GB_HTG2: | 167079 | AL096814 | Homo sapiens | Homo sapiens | 36,820 | 03-DEC-1999 |
| | | HSDJ139D8 | | | chromosome 6 clone | | | |
| | | | | | RP1-139D8 map | | | |
| | | | | | p12.1-21.1, | | | |
| | | | | | *** SEQUENCING | | | |
| | | | | | IN PROGRESS ***, | | | |
| | | | | | in unordered pieces. | | | |
| | | GB_HTG2: | 167079 | AL096814 | Homo sapiens | Homo sapiens | 36,820 | 03-DEC-1999 |
| | | HSDJ139D8 | | | chromosome 6 clone | | | |
| | | | | | RP1-139D8 map | | | |
| | | | | | p12.1-21.1, | | | |
| | | | | | ***SEQUENCING | | | |
| | | | | | IN PROGRESS ***, | | | |
| | | | | | in unordered pieces. | | | |
| | | GB_BA1: | 5461 | AB015853 | Pseudomonas | Pseudomonas | 39,121 | 13-Nov-98 |
| | | AB015853 | | | aeruginosa gene for | aeruginosa | | |
| | | | | | MexX and MexY, | | | |
| | | | | | complete cds. | | | |
| rxa02747 | 2199 | GB_BA1: | 5368 | AJ010319 | Corynebacterium | Corynebacterium | 100,000 | 14-MAY-1999 |
| | | CAJ10319 | | | glutamicum amtP, | glutamicum | | |
| | | | | | glnB, glnD genes | | | |
| | | | | | and partial ftsY | | | |
| | | | | | and srp genes. | | | |
| | | GB_GSS13: | 463 | AQ463737 | HS_5051_B2_D05_{—} | Homo sapiens | 37,549 | 23-Apr-99 |
| | | AQ463737 | | | SP6E RPCI-11 Human | | | |
| | | | | | Male BAC Library | | | |
| | | | | | Homo sapiens | | | |
| | | | | | genomic clone | | | |
| | | | | | Plate = | | | |
| | | | | | 627 Col = 10 | | | |
| | | | | | Row = H, | | | |
| | | | | | genomic survey | | | |
| | | | | | sequence. | | | |
| | | GB_BA1: | 5368 | AJ010319 | Corynebacterium | Corynebacterium | 100,000 | 14-MAY-1999 |
| | | CAJ10319 | | | glutamicum | glutamicum | | |
| | | | | | amtP, glnB, glnD | | | |
| | | | | | genes and | | | |
| | | | | | partial ftsY | | | |
| | | | | | and srp genes. | | | |
| rxa02760 | 1077 | GB_IN2: | 84551 | AC004295 | Drosophila | Drosophila | 40,303 | 29-Jul-98 |
| | | AC004295 | | | melanogaster DNA | melanogaster | | |
| | | | | | sequence | | | |
| | | | | | (P1 DS08374 (D180)), | | | |
| | | | | | complete sequence. | | | |
| | | GB_HTG6: | 141830 | AC011647 | Homo sapiens clone | Homo sapiens | 38,158 | 04-DEC-1999 |
| | | AC011647 | | | RP11-15D18, | | | |
| | | | | | ***SEQUENCING | | | |
| | | | | | IN PROGRESS ***, | | | |
| | | | | | 29 unordered pieces. | | | |
| | | GB_HTG6: | 141830 | AC011647 | Homo sapiens clone | Homo sapiens | 36,321 | 04-DEC-1999 |
| | | AC011647 | | | RP11-15D18, | | | |
| | | | | | ***SEQUENCING | | | |
| | | | | | IN PROGRESS ***, | | | |
| | | | | | 29 unordered pieces. | | | |
| rxa02787 | 1500 | GB_BA1: | 38807 | AL023591 | Mycobacterium | Mycobacterium | 57,533 | 27-Aug-99 |
| | | MLCB1259 | | | leprae cosmid B1259. | leprae | | |
| | | GB_BA1: | 38914 | L78820 | Mycobacterium | Mycobacterium | 57,600 | 15-Jun-96 |
| | | MSGB937CS | | | leprae cosmid B937 | leprae | | |
| | | | | | DNA sequence. | | | |
| | | GB_PR4: | 69718 | AC006474 | Homo sapiens clone | Homo sapiens | 37,246 | 1-Jul-99 |
| | | AC006474 | | | DJ0669I17, | | | |
| | | | | | complete sequence. | | | |
| rxa02830 | 662 | GB_BA1: | 31859 | Z83866 | Mycobacterium | Mycobacterium | 41,527 | 17-Jun-98 |
| | | MTCY22D7 | | | tuberculosis H37Rv | tuberculosis | | |
| | | | | | complete genome; | | | |
| | | | | | segment 133/162. | | | |
| | | GB_BA1: | 31859 | 283866 | Mycobacterium | Mycobacterium | 41,223 | 17-Jun-98 |
| | | MTCY22D7 | | | tuberculosis H37Rv | tuberculosis | | |
| | | | | | complete genome; | | | |
| | | | | | segment 133/162. | | | |
| | | GB_EST12: | 440 | AA276025 | vc30a07.r1 | Mus musculus | 38,746 | 1-Apr-97 |
| | | AA276025 | | | Barstead MPLRB1 | | | |
| | | | | | Mus musculus | | | |
| | | | | | cDNA clone IMAGE: | | | |
| | | | | | 776052 5′ | | | |
| | | | | | similar to gb: L38607 | | | |
| | | | | | Mus musculus | | | |
| | | | | | (MOUSE);, | | | |
| | | | | | mRNA sequence. | | | |
| rxa02831 | | | | | | | | |
| rxs03200 | 759 | GB_IN2: | 268984 | AE001274 | Leishmania major | Leishmania | 38,575 | 24-MAR-1999 |
| | | AE001274 | | | chromosome 1, | major | | |
| | | | | | complete sequence. | | | |
| | | GB_IN2: | 268984 | AE001274 | Leishmania major | Leishmania | 36,772 | 24-MAR-1999 |
| | | AE001274 | | | chromosome 1, | major | | |
| | | | | | complete sequence. | | | |
| | | GB_OM: | 5568 | X53085 | S. scrofa DNA for | Sus scrofa | 33,515 | 28-Jul-95 |
| | | SSIFNG | | | interferon-gamma. | | | |
| rxs03208 | 565 | GB_BA1: | 1091 | L35906 | Corynebacterium | Brevibacterium | 99,646 | 06-MAR-1996 |
| | | BRLDTXR | | | glutamicum | lactofermentum | | |
| | | | | | (clone pULJSX4) | | | |
| | | | | | diphtheria toxin | | | |
| | | | | | repressor (dtxr) gene, | | | |
| | | | | | complete cds. | | | |
| | | GB_BA1: | 38631 | Z96072 | Mycobacterium | Mycobacterium | 61,062 | 17-Jun-98 |
| | | MTCY05A6 | | | tuberculosis H37Rv | tuberculosis | | |
| | | | | | complete genome; | | | |
| | | | | | segment 120/162. | | | |
| | | GB_BA1: | 2604 | M80338 | Corynebacterium | Corynebacterium | 66,372 | 26-Apr-93 |
| | | CORDTXRAA | | | diphtheriae | diphtheriae | | |
| | | | | | diphtheria toxin | | | |
| | | | | | repressor (dtxR) | | | |
| | | | | | gene, complete | | | |
| | | | | | cds. | | | |
| rxs03219 | 1114 | GB_HTG3: | 200000 | AC005769 | Homo sapiens | Homo sapiens | 38,613 | 21-Aug-99 |
| | | AC005769 | | | chromosome 4, | | | |
| | | | | | *** SEQUENCING | | | |
| | | | | | IN PROGRESS ***, | | | |
| | | | | | 5 unordered pieces. | | | |
| | | GB_PR3: | 33189 | AF015723 | Homo sapiens | Homo sapiens | 36,866 | 21-Jan-98 |
| | | AF015723 | | | chromosome | | | |
| | | | | | 21q22 cosmid | | | |
| | | | | | clone Q4B12, | | | |
| | | | | | complete sequence. | | | |
| | | GB_HTG3: | 159747 | AC007315 | Homo sapiens clone | Homo sapiens | 35,005 | 23-Apr-99 |
| | | AC007315 | | | NH0189B16, | | | |
| | | | | | *** SEQUENCING | | | |
| | | | | | IN PROGRESS ***, | | | |
| | | | | | 3 unordered pieces. | | | |

1. An isolated nucleic acid molecule from Corynebacterium glutamicum encoding a metabolic pathway regulatory protein, or a portion thereof, provided that the nucleic acid molecule does not consist of any of the F-designated genes set forth in Table 1.

2. The isolated nucleic acid molecule of claim 1, Wherein said metabolic pathway regulatory protein is selected from the group consisting of proteins involved in the regulation of metabolism of organic acids, proteinogenic and nonproteinogenic amino acids, purine and pyrimidine bases, nucleosides, nucleotides, lipids, saturated and unsaturated fatty acids, diols, carbohydrates, aromatic compounds, vitamins, cofactors, and enzymes.

3. An isolated Corynebacterium glutamicum nucleic acid molecule selected from the group consisting of those sequences set forth in Appendix A, or a portion thereof, provided that the nucleic acid molecule does not consist of any of the F-designated genes set forth in Table 1.

4. An isolated nucleic acid molecule which encodes a polypeptide sequence selected from the group consisting of those sequences set forth in Appendix B, provided that the nucleic acid molecule does not consist of any of the F-designated genes set forth in Table 1.

5. An isolated nucleic acid molecule which encodes a naturally occurring allelic variant of a polypeptide selected from the group of amino acid sequences consisting of those sequences set forth in Appendix B, provided that the nucleic acid molecule does not consist of any of the F-designated genes set forth in Table 1.

6. An isolated nucleic acid molecule comprising a nucleotide sequence which is at least 50% homologous to a nucleotide sequence selected from the group consisting of those sequences set forth in Appendix A, or a portion thereof, provided that the nucleic acid molecule does not consist of any of the F-designated genes set forth in Table 1.

7. An isolated nucleic acid molecule comprising a fragment of at least 15 nucleotides of a nucleic acid comprising a nucleotide sequence selected from the group consisting of those sequences set forth in Appendix A, provides that the nucleic acid molecule does not consist of any of the F-designated genes set forth in Table 1.

8. An isolated nucleic acid molecule which hybridizes to the nucleic acid molecule of any one of claims 1-7 under stringent conditions.

9. An isolated nucleic acid molecule comprising the nucleic acid molecule of claim 1 or a portion thereof and a nucleotide sequence encoding a heterologous polypeptide.

10. A vector comprising the nucleic acid molecule of claim 1.

11. The vector of claim 10, which is an expression vector.

12. A host cell transfected with the expression vector of claim 11.

13. The host cell of claim 12, wherein said cell is a microorganism.

14. The host cell of claim 13, wherein said cell belongs to the genus Corynebacterium or Brevibacterium.

15. The host cell of claim 12, wherein the expression of said nucleic acid molecule results in the modulation in production of a fine chemical from said cell.

16. The host cell of claim 15, wherein said fine chemical is selected from the group consisting of: organic acids, proteinogenic and nonproteinogenic amino acids, purine and pyrimidine bases, nucleosides, nucleotides, lipids, saturated and unsaturated fatty acids, diols, carbohydrates, aromatic compounds, vitamins, cofactors, polyketides, and enzymes.

17. A method of producing a polypeptide comprising culturing the host cell of claim 12 in an appropriate culture medium to, thereby, produce the polypeptide.

18. An isolated metabolic pathway regulatory polypeptide from Corynebacterium glutamicum, or a portion thereof.

19. The protein of claim 18, wherein said polypeptide is selected from the group of metabolic pathway proteins which participate in the regulation of metabolism of organic acids, proteinogenic and nonproteinogenic amino acids, purine and pyrimidine bases, nucleosides, nucleotides, lipids, saturated and unsaturated fatty acids, diols, carbohydrates, aromatic compounds, vitamins, cofactors, and enzymes.

20. An isolated polypeptide comprising an amino acid sequence selected from the group consisting of those sequences set forth in Appendix B, provided that the amino acid sequence is not encoded by any of the F-designated genes set forth in Table 1.

21. An isolated polypeptide comprising a naturally occurring allelic variant of a polypeptide comprising an amino acid sequence selected from the group consisting of those sequences set forth in Appendix B, or a portion thereof, provided that the amino acid sequence is not encoded by any of the F-designated genes set forth in Table 1.

22. The isolated polypeptide of claim 18, further comprising heterologous amino acid sequences.

23. An isolated polypeptide which is encoded by a nucleic acid molecule comprising a nucleotide sequence which is at least 50% homologous to a nucleic acid selected from the group consisting of those sequences set forth in Appendix A, provided that the nucleic acid molecule does not consist of any of the F-designated nucleic acid molecules set forth in Table 1.

24. An isolated polypeptide comprising an amino acid sequence which is at least 50% homologous to an amino acid sequence selected from the group consisting of those sequences set forth in Appendix B, provided that the amino acid sequence is not encoded by any of the F-designated genes set forth in Table 1.

25. A method for producing a fine chemical, comprising culturing a cell containing a vector of claim 12 such that the fine chemical is produced.

26. The method of claim 25, wherein said method further comprises the step of recovering the fine chemical from said culture.

27. The method of claim 25, wherein said method further comprises the step of transfecting said cell with the vector of claim 11 to result in a cell containing said vector.

28. The method of claim 25, wherein said cell belongs to the genus Corynebacterium or Brevibacterium.

29. The method of claim 25, wherein said cell is selected from the group consisting of: Corynebacterium glutamicum, Corynebacterium herculis, Corynebacteriunm, lilium, Corynebacteriunm acetoacidophilum, Corynebacterium acetoglutamicum, Corynebacterium acetophilum, Corynebacterium ammoniagenes, Corynebacterium fujiokense, Corynebacterium nitrilophilus, Brevibacterium ammoniagenes, Brevibacterium butanicum, Brevibacterium divaricatum, Brevibacterium flavum, Brevibacterium healii, Brevibacterium ketoglutamicum, Brevibacterium ketosoreductum, Brevibacterium lactofermentum, Brevibacterium linens, Brevibacterium paraffinolyticum, and those strains set forth in Table 3.

30. The method of claim 25, wherein expression of the nucleic acid molecule from said vector results in modulation of production of said fine chemical.

31. The method of claim 25, wherein said fine chemical is selected from the group consisting of: organic acids, proteinogenic and nonproteinogenic amino acids, purine and pyrimidine bases, nucleosides, nucleotides, lipids, saturated and unsaturated fatty acids, diols, carbohydrates, aromatic compounds, vitamins, cofactors, polyketides, and enzymes.

32. The method of claim 25, wherein said fine chemical is an amino acid.

33. The method of claim 32, wherein said amino acid is drawn from the group consisting of: lysine, glutamate, glutamine, alanine, aspartate, glycine, serine, threonine, methionine, cysteine, valine, leucine, isoleucine, arginine, proline, histidine, tyrosine, phenylalanine, and tryptophan.

34. A method for producing a fine chemical, comprising culturing a cell whose genomic DNA has been altered by the inclusion of a nucleic acid molecule of any one of claims 1-9.

35. A method for diagnosing the presence or activity of Corynebacterium diphtheriae in a subject, comprising detecting the presence of one or more of the sequences set forth in Appendix A or Appendix B in the subject, provided that the sequences are not or are not encoded by any of the F-designated sequences set forth in Table 1, thereby diagnosing the presence or activity of Corynebacterium diphtheriae in the subject.

36. A host cell comprising a nucleic acid molecule selected from the group consisting of the nucleic acid molecules set forth in Appendix A, wherein the nucleic acid molecule is disrupted.

37. A host cell comprising a nucleic acid molecule selected from the group consisting of the nucleic acid molecules set forth in Appendix A, wherein the nucleic acid molecule comprises one or more nucleic acid modifications from the sequence set forth in Appendix A.

38. A host cell comprising a nucleic acid molecule selected from the group consisting of the nucleic acid molecules set forth in Appendix A, wherein the regulatory region of the nucleic acid molecule is modified relative to the wild-type regulatory region of the molecule.
